(19) United States
(12) Patent Application Publication
(10) Pub. No.: US 2003/0096399 A1
(43) Pub. Date: May 22, 2003

(54) Substantially complete ribozyme libraries

(75) Inventors: Jack Barber, San Diego, CA (US); Peter Welch, San Diego, CA (US); Xinqiang Li, San Diego, CA (US); Richard Tritz, San Diego, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Immusol Incorporated, San Diego, CA

(21) Appl. No.: 10/067,956

(22) Filed: Feb. 5, 2002

Related U.S. Application Data
(63) Continuation of application No. 09/357,741, filed on Jul. 20, 1999.
(60) Provisional application No. 60/093,828, filed on Jul. 22, 1998.

(51) Int. Cl. C12N 15/00; C12N 15/63; C12N 15/74

(52) U.S. Cl. 435/320.1

(57) ABSTRACT

The present invention provides a high complexity substantially complete hairpin ribozyme library having a randomized recognition sequence, packaged in a vector and operably linked to a promoter suitable for high level expression in a wide variety of cells. The invention comprises using the library in a variety of selection protocols for identifying, isolating and characterizing known or unknown target RNAs, to reveal the phenotypic effects of such cleavage, and to identify the gene products that produce those phenotypic effects.

### CROSS-REFERENCE TO RELATED APPLICATIONS

This application is a continuation in part of U.S. patent application Ser. NO. 60/093,828, filed Jul. 22, 1998.

### STATEMENT AS TO RIGHTS TO INVENTIONS MADE UNDER FEDERALLY SPONSORED RESEARCH AND DEVELOPMENT

[Not Applicable]

1. Field of the Invention

This invention relates generally to methods for using hairpin ribozymes in functional genomics. In particular, this invention provides substantially complete ribozyme libraries and methods of using such libraries for identifying, isolating, and characterizing unknown genes and gene products. The libraries are also useful in methods of assigning function to known genes. Compared to other known ribozymes, the hairpin ribozyme has been discovered to be uniquely effective as a randomized antisense tool.

2. Background of the Invention

A ribozyme is an RNA molecule that catalytically cleaves other RNA molecules. Different kinds of ribozymes have been described, including group I ribozymes, hammerhead ribozymes, hairpin ribozymes, RNAse P, and axhead ribozymes. See Castanotto et al. (1994) Advances in Pharmacology 25:289-317 for a general review of the properties of different ribozymes.

The general features of hairpin ribozymes are described e.g., in Hampel et al. (1990) Nucl. Acids Res. 18:299-304; Hampel et al. (1990) European Patent Publication No. 0 360 257; U.S. Pat. No. 5,254,678, issued Oct. 19, 1993; Wong-Staal et al., WO 94/26877; Ojwang et al. (1993) Proc. Natl. Acad. Sci. USA 90:6340-6344; Yamada et al. (1994) Human Gene Therapy 1:39-45; Leavitt et al. (1995) Proc. Natl. Acad. Sci. USA 92:699-703; Leavitt et al. (1994) Human Gene Therapy 5:1151-1120; and Yamada et al. (1994) Virology 205:121-126. Hairpin ribozymes typically cleave one of two target sequences, NNNNN*GUCNNNNNNNN or NNNNN*GUANNNNNNNN where “*” denotes the cleavage site, and N can be any nucleotide (see, De Young et al. (1995) Biochemistry 34:15785-15791). The products of the cleavage reaction are a5′ fragment terminating in a 2′,340 cyclic phosphate and a 3′ fragment bearing a newly formed 5′—OH. The reaction is reversible; ribozymes also catalyze the formation of phosphodiester bonds (see generally, Buzayan et al. (1986) Nature 323:349-352; Gerlach et al. (1986) Virology 151:172-185; Hampel et al. (1989) Biochemistry 28:4929-4933; Gerlach et al. (1989) Gene 82:43-52; Feldstein et al. (1989) Gene 82:53-61; and Hampel et al. Australian Patent No. AU-B-41594/89).

Ribozymes can be used to engineer RNA molecules prior to reverse transcription and cloning, in a manner similar to the DNA endonuclease “restriction” enzymes. The production of specific ribozymes which target particular sequences is taught in the art (see, e.g., Yu et al. (1993) Proc. Natl. Acad. Sci. USA 90:6340-6344 and Dropulic et al. (1992) J. Virol. 66(3):1432-1441; Wong-Staal et al., WO 94/26877). Ribozymes which cleave or ligate a particular RNA target sequence can be expressed in cells to prevent or promote expression and translation of RNA molecules comprising the target sequence.

For instance, expression of hairpin ribozymes which specifically cleave human immunodeficiency (HIV) RNAs prevent replication of the virus in cells. See, Yu et al. (1993) Proc. Natl. Acad. Sci. USA 90:6340-6344; Yamada et al. (1994) Virology 205:121-126; Yamada et al. (1994) Gene Therapy 1:38-45; Yu et al. (1995) Virology 206:381-386; Yu et al. (1995) Proc. Nat. Acad. Sci. 92:699-703; and Wong-Staal et al. WO 94/26877 (PCT/US94/05700). The trans-splicing activity of ribozymes can be used to repair defective mRNA transcripts within cells and restore gene expression. Sullenger and Cech (1994) Nature 371:619-622. Quasi-random ribozyme expression vectors were reportedly used to clone target specific ribozymes. Macjak and Draper (1993) J. Cell. Biochem. Supplement 17E, S206:202. A hammerhead ribozyme library comprising a randomized recognition sequence was used for in vitro selection of ribozymes which actively cleave a specific target RNA (Lieber and Strauss (1995) Mol. Cell. Biol. 15:540-551; patent publication 96/01314); ribozymes selected by this method were then expressed in tissue culture cells (id.) and in transgenic mice (Lieber and Kay (1996) J. Virol. 70:3153-3158). In addition, hammerhead ribozyme libraries comprising a randomized catalytic region have been used to select ribozymes that efficiently cleave a specific target RNA. Patent publication WO 92/01806. A library of the ribozyme form of the group I intron of Tetrahymena thermophila having a partially randomized recognition sequence was used for in vitro selection of ribozymes which actively cleave a specific target RNA. Campbell and Cech (1995) RNA 1:598-609.

However, even when both the sequence of the cleavage sites of a specific target RNA and the recognition sequences of ribozymes that cleave that specific RNA are known, targeted cleavage of RNA in vivo has been difficult to achieve (See, e.g., Ojwang et al. (1992) Proc. Natl. Acad. Sci. USA 89:10802-10806), in part for the following reasons: (a) The target site may be hidden within the folds of secondary structure in the substrate RNA, or by interaction with RNA binding molecules. (b) The substrate RNA and the ribozyme may not be present in the same cellular compartment. (c) The ribozyme may be inhibited or inactivated in vivo, either because it is degraded, or because it assumes a secondary structure in vivo that is incompatible with catalytic activity, or because of interactions with cellular molecules. The observed biological effects in some instances can be attributed to simple binding of the ribozyme, as opposed to binding and cleavage. (d) The ribozyme is not produced in sufficient quantities.

These difficulties become even more pronounced when ribozyme libraries (e.g. collections of randomized ribozymes) are used in a selection protocol to isolate particular binding ribozymes. In this context because the libraries are essentially random, the ribozymes are not optimized for a particular target.

The present invention addresses these and other problems.

### SUMMARY OF THE INVENTION

This invention provides novel substantially complete ribozyme libraries that are suitable for use in a multitude of applications, in particular for target acquisition. Because the ribozyme libraries of this invention are complete or substantially complete libraries of high complexity, the likelihood of identifying a ribozyme or multiple ribozymes that specifically bind a particular target is vastly increased and the problems associated with the use of non-optimal ribozymes in a screening system are thereby overcome.

In one embodiment, this invention provides a substantially complete ribozyme library comprising a collection of adeno-associated virus (AAV) vectors, or a collection of retroviral vectors containing nucleic acids encoding hairpin ribozymes in expression cassettes wherein said collection of AAV vectors or collection of retroviral vectors contains nucleic acids encoding on average about 90% or more of all possible hairpin ribozyme binding sequences having eight or more randomized nucleotides. In one particularly preferred ribozyme library the collection of AAV vectors or collection of retroviral vectors contains nucleic acids encoding on average about 95% or more of all possible hairpin ribozyme binding sequences. In another the collection of AAV vectors or collection of retroviral vectors contains nucleic acids encoding on average about 95% or more of all possible hairpin ribozyme binding sequences having 9 or more randomized nucleotides. In still another ribozyme library, the collection of AAV vectors or collection of retroviral vectors contains nucleic acids encoding about 95% or more of all possible hairpin ribozyme binding sequences having 12 randomized nucleotides. In a preferred ribozyme library, the nucleic acids are plasmids.

In another embodiment, this invention also provides for a substantially complete ribozyme gene library comprising a collection of plasmids wherein members of said collection encode a retroviral or adeno-associated virus (AAV) vector containing a ribozyme-encoding nucleic acid and said collection of plasmids encodes on average about 90% or more of all possible hairpin ribozyme binding sequences having eight or more randomized nucleotides. In one particularly preferred ribozyme gene library the collection of plasmids encodes on average about 95% or more of all possible hairpin ribozyme binding sequences. In another ribozyme gene library, the collection of plasmids encodes on average about 95% or more of all possible hairpin ribozyme binding sequences having 9 or more randomized nucleotides. In still another ribozyme gene library, the collection plasmids contains nucleic acids encoding on average about 95% or more of all possible hairpin ribozyme binding sequences having 12 randomized nucleotides.

In another embodiment, in either the ribozyme library or the ribozyme gene library, the library contains no toxic ribozymes. In preferred libraries, the vector is an AAV vector. The nucleic acids comprising the ribozyme library or ribozyme gene library can comprise a pair of inverted terminal repeats (ITRs) of adeno-associated viral genome. A selectable marker (e.g., Neo^{r}, amd Hydro^{r}) may be present, and can be operably linked to an SV40 promoter. The ribozyme-encoding nucleic acid can be operably linked to a tRNA promoter (e.g., tRNAval, tRNAser) or other promoters such as a PGK promoter.

In another embodiment, this invention provides methods of selecting a ribozyme that specifically binds and cleaves a nucleic acid target. The methods involve transfecting a population of cells with a substantially complete hairpin ribozyme library as described herein, detecting a phenotypic difference between a transfected cell that expresses at least one hairpin ribozyme encoded by said library and a control cell lacking an active member of the ribozyme library, wherein the phenotypic difference is a consequence of cleavage of said target; and recovering a ribozyme associated with the phenotypic difference. In one embodiment, the transfection produces a population of cells stably transfected with an expression cassette encoding a hairpin ribozyme. The hairpin ribozyme may be constitutively expressed in the cells. Recovery of the ribozyme can comprise isolating a multiplicity of ribozymes to produce a targeted ribozyme library. The targeted library can then be used to transfect a population of cells with said targeted ribozyme library. A phenotypic difference is then detected between a transfected cell that expresses at least one hairpin ribozyme encoded by said targeted ribozyme library and a control cell lacking an active member of said ribozyme library, wherein said phenotypic difference is a consequence of cleavage of the target. The ribozyme(s) associated with said phenotypic difference are then recovered.

This invention also provides methods of identifying a gene or mRNA altered expression of which results in alteration of a detectable phenotypic character. The methods involve i) stably transfecting a population of cells with a hairpin ribozyme library comprising a collection of adeno-associated virus (AAV) vectors containing nucleic acids encoding hairpin ribozymes in expression cassettes; ii) detecting a phenotypic difference between a transfected cell that expresses said hairpin ribozyme and a control cell lacking an active form of said hairpin ribozyme; iii) recovering a ribozyme associated with said phenotypic difference; and iv) sequencing the binding site sequence of the recovered ribozyme to identify the nucleic acid to which it bound. The hairpin ribozyme may be constitutively expressed. In one embodiment, the hairpin ribozyme library can be any of the substantially complete ribozyme libraries or ribozyme gene libraries of this invention or alternatively can be a targeted library.

In the methods described herein the recovery of the ribozyme can involve isolating and sequencing the binding site of the ribozyme(s). The method can further involve providing a probe (e.g., a labeled probe) that hybridizes to the nucleic acid specifically bound by said ribozyme. In the methods described herein, the phenotypic difference may include, but is not limited to a difference in transcription or expression of a reporter gene or cDNA, the ability of a cell to grow on soft agar, the ability of a cell to differentiate (e.g. as identified by the adherence of the cell to a surface in culture), resistance to a drug (e.g. cytotoxic drug such as cisplatin, doxirubicin, taxol, camptothecin, daunorubicin, methotrexate, etc.), or a change in the expression level of a reporter gene linked to a gene whose regulation it is desired to alter.

In still another embodiment, this invention provides methods of producing a cell line having altered expression of a gene. The methods involve stably transfecting a cell with a vector encoding a hairpin ribozyme wherein said hairpin ribozyme is identified according to the screening methods (e.g. screening of a substantially complete ribozyme library) described herein.

This invention also provide population of mammalian cells containing (e.g. stably expressing) any of the substantially complete ribozyme libraries described herein.

This invention also provides kits for practice of any of the methods described herein. The kits preferably comprise one or more containers containing a substantially complete ribozyme or a substantially complete ribozyme gene library as described herein.

Definitions

A “ribozyme sequence tag” or “RST” is the complementary sequence of the target RNA specifically recognized by the binding site of a ribozyme.

The term ribozyme library generally refers to a collection of ribozymes or a collection of molecules encoding ribozymes. In a preferred embodiment, this invention contemplates two types of “ribozyme library”; a “ribozyme gene library” and a “ribozyme vector library.” A “ribozyme gene library” is a collection of ribozyme-encoding genes that, when transcribed produce, ribozymes. The genes are typically contained within expression cassettes and the library is typically maintained as a plasmid (or other equivalent construct, e.g., cosmid, phagemid, etc.) that can be maintained and amplified, typically in bacterial (e.g. E. coli) culture. Preferred ribozyme gene libraries encode a vector sequence containing the ribozyme encoding nucleic acid and are referred to as a provector. A “ribozyme vector library” is collection of ribozyme-encoding genes, typically within expression cassettes, in a collection of viral vectors. The viral vectors may be naked or contained within a capsid. The viral vectors are typically maintained and/or propagated in mammalian cell culture.

The term “nucleic acid” refers to deoxyribonucleotides or ribonucleotides and polymers thereof, in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless otherwise indicated by the usage of the term, the term nucleic acid is often used interchangeably with gene, cDNA, and mRNA encoded by a gene.

The phrase “a nucleic acid sequence encoding” refers to a nucleic acid which contains sequence information for a structural RNA such as rRNA, a tRNA, or the primary amino acid sequence of a specific protein or peptide, or a binding site for a trans-acting regulatory agent. Unless otherwise indicated, a particular coding nucleic acid sequence also implicitly encompasses conservatively modified variants thereof (e.g. degenerate codon substitutions) and complementary sequences, as well as the sequence explicitly indicated. Specifically, degenerate codon (i.e., different codons which encode a single amino acid) substitutions may be achieved by generating sequences in which the third position of one or more (or all) selected codons is substituted with mixed-base and/or deoxyinosine residues (Batzer et al. (1991) Nucleic Acid Res. 19:5081; Ohtsuka et al. (1985) J. Biol. Chem. 260:2605-2608; and Cassol et al. (1992); Rossolini et al. (1994) Mol. Cell. Probes 8:91-98). Degenerate codons of the native sequence or sequences that may be introduced to conform with codon preference in a specific host cell.

The term “sub-sequence” in the context of a particular reference nucleic acid refers to a region of the nucleic acid smaller than the reference nucleic acid or polypeptide.

“Cellular gene” means a gene usually expressed by the members of a given cell line or cell type without experimental manipulation. It preferably means an endogenous gene that forms part of the cellular genome. Genes expressed by intracellular parasites (e.g. bacteria, viruses, etc.) that may be adventitously expressed in a particular cell or cell line are considered “cellular genes”. However, the term specifically excludes genes that are expressed in a particular population of cells due to the deliberate experimental infection of that population with selected viruses.

A “ribozyme” is a catalytic RNA molecule which cleaves RNA. The preferred class of ribozymes for the invention is the hairpin ribozyme; hammerheads are specifically not preferred. Preferred hairpin ribozymes cleave target RNA molecules in trans. A ribozyme cleaves a target RNA in vitro when it cleaves a target RNA in solution. A ribozyme cleaves a target RNA in vivo when the ribozyme cleaves a target RNA in a cell. The cell is optionally isolated, or present with other cells, e.g., as part of a tissue, tissue extract, cell culture, or live organism. For example, a ribozyme is active in vivo when it cleaves a target RNA in a cell present in an organism such as a mammal, or when the ribozyme cleaves a target RNA in a cell present in cells or tissues isolated from a mammal, or when it cleaves a target RNA in a cell in a cell culture.

A ribozyme “recognition sequence” is the portion of a nucleic acid encoding the ribozyme which is complementary to a target RNA. Upon binding of the ribozyme to the target RNA via this recognition sequence, two regions of double-stranded RNA are formed, termed “helix 1” and “helix 2.” A GUC ribozyme typically cleaves an RNA having the sequence 5′-NNNNN*GUCNNNNNNNN (SEQ ID NO:1) (where N*G is the cleavage site and where N is any of G, U, C, or A) where helix 1 is defined as the 6 to 10 bases 3′ of the GUC and helix 2 is defined as the 4 bases 5′ of the GUC. GUA ribozymes typically cleave an RNA target sequence consisting of NNNNN*GUANNNNNNNN. (SEQ ID NO:2) (where N*G is the cleavage site and where N is any of G, U, C, or A). A “GUA site” is an RNA sub-sequence that includes the nucleic acids GUA which is cleaved b a GUA ribozyme. A “GUC site” is an RNA sub-sequence which includes the nucleic acids GUC which is cleaved by a GUC ribozyme. A library of GUC hairpin ribozyme-encoding genes will therefore have the subsequence 5′-(N)₍₆₋₁₀₎AGAA(N)₄3′, where N can be either G, T, C, or A.

The term “isolated”, when applied to a nucleic acid or protein, denotes that the nucleic acid or protein is essentially free of other cellular components with which it is associated in the natural state. In particular, an isolated gene of interest is separated from open reading frames which flank the gene and encode a gene product other than that of the specific gene of interest. A “purified” nucleic acid or protein gives rise to essentially one band in an electrophoretic gel, and is at least 85% pure, more preferably at least 95% pure, and most preferably at least 99% pure.

“Nucleic acid probes” may be DNA or RNA fragments. DNA fragments can be prepared, for example, by digesting plasmid DNA, or by use of PCR, or synthesized by either the phosphoramidite method described by Beaucage and Carruthers (1981) Tetrahedron Lett. 22:1859-1862, or by the triester method according to Matteucci et al. (1981) J. Am. Chem. Soc., 103:3185, both incorporated herein by reference. A double stranded fragment may then be obtained, if desired, by annealing the chemically synthesized single strands together under appropriate conditions or by synthesizing the complementary strand using DNA polymerase with an appropriate primer sequence. Where a specific sequence for a nucleic acid probe is given, it is understood that the complementary strand is also identified and included. The complementary strand will work equally well in situations where the target is a double-stranded nucleic acid.

The phrase “selectively hybridizing to” refers to a nucleic acid probe that hybridizes, duplexes or binds only to a particular target DNA or RNA sequence when the target sequences are present in a preparation of, for example, total cellular DNA or RNA. “Complementary” or “target” nucleic acid sequences refer to those nucleic acid sequences which selectively hybridize to a nucleic acid probe. Proper annealing conditions depend, for example, upon a probe's length, base composition, and the number of mismatches and their position on the probe, and must often be determined empirically. For discussions of nucleic acid probe design and annealing conditions, see, for example, Sambrook et al. (1989) Molecular Cloning: A Laboratory Manual (2d ed.), Vols. 1-3, Cold Spring Harbor Laboratory (hereinafter, Sambrook et al.) or F. Ausubel et al. (ed.) (1987) Current Protocols in Molecular Biology, Greene Publishing and Wiley-Interscience, New York (1987).

A “promoter” is an array of cis-acting nucleic acid control sequences which direct transcription of an associated nucleic acid. As used herein, a promoter includes nucleic acid sequences near the start site of transcription, such as a polymerase binding site. The promoter also optionally includes distal enhancer or repressor elements which can be located as much as several thousand base pairs from the start site of transcription. A “constitutive” promoter is a promoter which is active under most environmental conditions and states of development or cell differentiation, such as a pol III promoter. An “inducible” promoter initiates transcription in response to an extracellular stimulus, such as a particular temperature shift or exposure to a specific chemical.

A “pol III promoter” is a DNA sequence competent to initiate transcription of associated DNA sequences by pol III. Many such promoters are known, including those which direct expression of known t-RNA genes. A general review of various t-RNA genes can be found in Watson et al. Molecular Biology of The Gene Fourth Edition, The Benjamin Cummings Publishing Co., Menlo Park, Calif. pages 710-713.

A nucleic acid of interest is “operably linked” to a promoter, vector or other regulatory sequence when there is a functional linkage in cis between a nucleic acid expression control sequence (such as a promoter, or array of transcription factor binding sites) and the nucleic acid of interest. In particular, a promoter that is operably linked to a nucleic acid of interest directs transcription of the nucleic acid.

A regulatory nucleic acid is one that initiates, causes, enhances or inhibits the expression of a particular selected nucleic acid or gene product, either directly or through its gene product. Examples of trans-acting regulatory nucleic acids includes nucleic acids that encode initiators, inhibitors and enhancers of transcription, translation, or post-transcriptional (e.g., RNA splicing factors) or post translational processing factors, kinases, proteases

An “expression vector” includes a recombinant expression cassette which has a nucleic acid which encodes an RNA that can be transcribed by a cell. A “recombinant expression cassette” is a nucleic acid construct, generated recombinantly or synthetically, with a series of specified nucleic acid elements which permit transcription of an encoded nucleic acid in a target cell. The expression vector can be part of a plasmid, virus, or nucleic acid fragment. Typically, the recombinant expression cassette portion of an expression vector includes a nucleic acid to be transcribed, and a promoter. In some embodiments, the expression cassette also includes, e.g., an origin of replication, and/or chromosome integration elements such as retroviral LTRs, or adeno associated viral (AAV) ITRs.

The phrase “exogenous,” “genetically engineered” or “heterologous nucleic acid” generally denotes a nucleic acid that has been isolated, cloned and ligated to a nucleic acid with which it is not combined in nature, and/or introduced into and/or expressed in a cell or cellular environment other than the cell or cellular environment in which said nucleic acid or protein may typically be found in nature. The term encompasses both nucleic acids originally obtained from a different organism or cell type than the cell type in which it is expressed, and also nucleic acids that are obtained from the same cell line as the cell line in which it is expressed. The term also encompasses a nucleic acid indicates that the nucleic acid comprises two or more subsequences which are not found in the same relationship to each other in nature. For instance, the nucleic acid is typically recombinantly produced, having two or more sequences derived from unrelated genes arranged to make a new functional nucleic acid. For example, in one embodiment, the nucleic acid has a promoter from one gene, such as a human t-RNA gene, arranged to direct the expression of a coding sequence from a different gene, such as an artificial gene coding for a ribozyme. When used with reference to a ribozyme, the term “heterologous” means that the ribozyme is expressed in a cell or location where it is not ordinarily expressed in nature, such as in a T cell which encodes the ribozyme in an expression cassette.

The term “recombinant” or “genetically engineered” when used with reference to a nucleic acid or a protein generally denotes that the composition or primary sequence of said nucleic acid or protein has been altered from the naturally occurring sequence using experimental manipulations well known to those skilled in the art. It may also denote that a nucleic acid or protein has been isolated and cloned into a vector or a nucleic acid that has been introduced into or expressed in a cell or cellular environment, particularly in a cell or cellular environment other than the cell or cellular environment in which said nucleic acid or protein may be found in nature.

The term “recombinant” or “genetically engineered” when used with reference to a cell indicates that the cell replicates or expresses a nucleic acid, or produces a peptide or protein encoded by a nucleic acid, whose origin is exogenous to the cell. Recombinant cells can express nucleic acids that are not found within the native (nonrecombinant) form of the cell. Recombinant cells can also express nucleic acids found in the native form of the cell wherein the nucleic acids are re-introduced into the cell by artificial means.

A cell has been “transduced” or “transfected” by an exogenous nucleic acid when such exogenous nucleic acid has been introduced inside the cell membrane. Exogenous DNA may or may not be integrated (covalently linked) into chromosomal DNA making up the genome of the cell. The exogenous DNA may be maintained on an episomal element, such as a plasmid. In eukaryotic cells, a stably transformed cell is generally one in which the exogenous DNA has become integrated into the chromosome so that it is inherited by daughter cells through chromosome replication, or one which includes stably maintained extrachromosomal plasmids. This stability is demonstrated by the ability of the eukaryotic cell to establish cell lines or clones comprised of a population of daughter cells containing the exogenous DNA.

A vector “transduces” a cell when it transfers nucleic acid into the cell. A cell is “stably transduced” by a nucleic acid when a nucleic acid transduced into the cell becomes stably replicated by the cell, either by incorporation of the nucleic acid into the cellular genome, or by episomal replication. A vector is “infective” when it transduces a cell, replicates, and (without the benefit of any complementary vector) spreads progeny vector of the same type as the original transducing vector to other cells in an organism or cell culture, wherein the progeny vectors have the same ability to reproduce and spread throughout the organism or cell culture.

The phrase “specifically binds to an antibody” or “specifically immunoreactive with”, when referring to a protein or peptide, refers to a binding reaction which is determinative of the presence of the protein in the presence of a heterogeneous population of proteins and other biologics. Thus, under designated immunoassay conditions, the specified antibodies bind to a particular protein and do not bind in a significant amount to other proteins present in the sample. Specific binding to an antibody under such conditions may require an antibody that is selected for its specificity for a particular protein. A variety of immunoassay formats may be used to select antibodies specifically immunoreactive with a particular protein. For example, solid-phase ELISA immunoassays are routinely used to select monoclonal antibodies specifically immunoreactive with a protein. See Harlow and Lane (1988) Antibodies, A Laboratory Manual, Cold Spring Harbor Publications, New York, for a description of immunoassay formats and conditions that can be used to determine specific immunoreactivity.

A “transgene” comprises a nucleic acid sequence used to form a chimeric or transgenic animal when introduced into the chromosomal material of the somatic and germ line cells of a non-human animal by way of human intervention, such as by way of the methods described herein to form a transgenic animal. The particular embodiments of the transgenes of the invention are described in more detail hereinafter.

An “embryonic target cell” is a cell into which the transgenes of the invention are introduced to produce “chimeric” animals (wherein only a subset of cells is transduced) or “transgenic” non-human animals (wherein every cell is transduced). Examples include embryonic stem (ES) cells, or preferably the fertilized oocyte (zygote). In some cases, chimeric animals can also be produced by isolating stem cells from an animal, transducing them in vitro, and reinfusing them into the original donor or into an allogeneic recipient.

“Expresses” denotes that a given nucleic acid comprising an open reading frame is transcribed to produce an RNA molecule. It also denotes that a given nucleic acid is transcribed and translated to produce a polypeptide. “Gene product” refers to the RNA produced by transcription or to the polypeptide produced by translation of a nucleic acid. It will be recognized, however, that the term expresses is sometimes used to refer to the transcription of a ribozyme. The ribozyme is active (catalytic) as a nucleic acid and is typically not translated into a protein. The difference in usage of the term “expresses” or “expression” will be apparent from context.

“Cloning a cell” denotes that a single cell is proliferated to produce a genetically and phenotypically homogeneous population of progeny cells descended from the single cell.

A “ligand” is a molecule or chemical compound that detectably and selectively binds to a reference molecule but not to other molecules, preferably with an affinity higher than 10⁻³ M, more preferably greater than 10⁻⁵ M, and most preferably about 10⁻⁷ or higher.

“Sensitivity to a selected chemical compound” means that exposure to a particular chemical compound reproducibly causes a cell to alter its metabolism in predictable ways, e.g. by inducing slower growth, apoptosis, proliferation, induction o or shutdown of certain genes, etc.

“Packaging” or “packaged” denotes that a specific nucleic acid or library is contained in and operably linked to a defined vector, such as an adenovirus associated vector.

The “complexity” or “diversity” of a library refers to the number of different ribozyme members present in that library.

The phrase “encoding on average about X% or more of all possible hairpin ribozyme binding sequences” is intended to recognize that when dealing with populations of nucleic acids, vectors, etc. it is not possible to guarantee that every single member of the population is present in any particular experiment. It is also extremely difficult (virtually impossible) to directly count all of the different members of a complex library. However, it can be determined, e.g. using the equations provided herein, how large and diverse a library must be to include the desired number of members at a certain level of confidence (statistical probability). Thus a library encoding on average about X% or more of all possible hairpin ribozyme binding sequences encodes X% of all possible hairpin ribozyme binding sequences with a probability of better than 90%, preferably better than 95%, more preferably better than 98% and most preferably better than 99%.

“Phenotype” denotes a definable detectable heritable trait of a cell or organism, that is caused by the presence and action at least one gene. The terms “phenotype”, “phenotypic character”, and “biological activity” may be used interchangeably herein to refer to a measurable (detectable) property of a cell or cells, tissue, organ, or organism. Such a character can include, but is not limited to, a morphological trait, an enzymatic activity, a motility, and the like.

When a library is said to contain no toxic ribozymes, the library generally lacks ribozymes that when present in a normal healthy mammalian cell induce death of that cell under normal culture conditions. Preferred high complexity libraries containing no toxic ribozymes contain on average less than about 5%, preferably less than about 2%, more preferably less than about 1%, and most preferably less than about 0.1% toxic ribozymes, a particularly preferred library, on average contains no toxic ribozymes.

The term “plasmid” as used herein includes plasmids and similar vectors typically used for cloning various genes. Such vectors include, but are not limited to plasmids, phagemids, cosmids, etc.

The term “tetraloop” refers to a stabilizing modification of loop 3 of the hairpin ribozyme. The standard GUU loop 3 of the hairpin ribozyme (Hampel et al. (1990) Nucl. Acids Res. 18: 299-304) is replaced by a 12 nucleotide tetraloop sequence, 5′-GGAC(UUCG)GUCC-3′ (SE ID NO:_), commonly found in cellular RNA structures. The resulting tetraloop ribozyme has a 7 bp helix 4 (versus 3 in the conventional hairpin ribozyme) and a new UUCG sequence in loop 3. The tetraloop forms a very stable structure which simultaneously enhances the stability of the ribozyme and decreases the size of loop 3, which is otherwise exposed to cellular nucleases.

### BRIEF DESCRIPTION OF THE DRAWINGS

FIG. 1 illustrates the hairpin ribozyme. The hairpin ribozyme consists of a 50 to 54 nucleotide RNA molecule (shaded, in uppercase letters) which binds and cleaves an RNA substrate (lowercase letters). The catalytic RNA folds into a 2-dimensional structure that resembles a hairpin, consisting of two helical domains (Helix 3 and 4) and 3 loops (Loop 2, 3 and 4). Two additional helixes, Helix 1 and 2, form between the ribozyme and its substrate. Recognition of the substrate by the ribozyme is via Watson-Crick base pairing (where N or n=any nucleotide). The length of Helix 2 is fixed at 4 basepairs and the length of Helix 1 typically varies from 6 to 10 basepairs. The substrate contains a GUC in Loop 5 for maximal activity, and cleavage occurs immediately 5′ of the G as indicated by an arrow. The catalytic, but not substrate binding, activity of the ribozyme can be disabled by mutating the AAA in Loop 2 to CGU.

FIG. 2 shows a schematic of trans cleavage and ligation. The auto-catalytic ribozyme library is transcribed in vitro and allowed to self-cleave. Self-cleaved, helix 2-charged ribozymes are purified and incubated with the target RNA. Following cleavage of target, a portion of the charged ribozymes will ligate themselves to the cleavage products. These product-ribozyme species are then amplified by reverse transcription and PCR to yield the target specific ribozymes.

FIG. 3 illustrates the immobilization or target RNA on solid supports by either their 5′ or 3′ ends.

FIG. 4 illustrates the in vitro selection of efficient ribozymes. An in vitro transcribed ribozyme library is applied to the target RNA column under conditions that allow binding but prevent cleavage. Unbound ribozyme are washed away. Conditions are changed to allow cleavage by the bound ribozymes. Active ribozymes are released from the column following successful cleavage. Released ribozyme are amplified, re-synthesized and re-applied to a new column and the process is repeated.

FIG. 5 illustrates the PCR cloning scheme for production of a high complexity ribozyme gene library.

FIG. 6 illustrates the cleavage of various target RNAS with an AAV ribozyme vector.

FIG. 7 illustrates the vector p1014-2k

FIG. 8 illustrates the oligonucleotide ligation scheme for the production of pAAV6Clib with 7 random nucleotides in the helix 1 region driven by the tRNAval promoter.

FIG. 9 illustrates plasmid pAAVhygro-PGK.

FIG. 10 illustrates plasmid pPolII/PGKmus/neoBHGPA.

FIG. 11 illustrates plasmid p1015.

FIG. 12 illustrates the Scheme for the construction of ERL030398

FIGS. 13a and 13b illustrate plasmid vectors pLHPM-2 kb and pLPR-2 kb, respectively.

FIG. 14 illustrates the ligation scheme for the construction of Construction of retroviral plasmid ribozyme library

FIG. 15 shows an example of retroviral titer yields, represented as neomycin resistant colony forming units per milliliter.

FIG. 16 shows the effect of transfection with a ribozyme library on cisplatin resistance of cells in culture.

FIG. 17 illustrates the identification of a cellular target gene using a biotinylated ribozyme sequence tag identification

FIG. 18 illustrates a construct having the BRCA-1 promoter region cloned in front of the selection marker EGFP (enhanced green fluorescent protein).

FIG. 19 illustrates the BRCA-1 promoter replaced with the CMV promoter, thus allowing deregulated, constitutive EGFP expression as a control for the construct of FIG. 18.

FIG. 20 shows a comparison of BRCA-1 and CMV promoters in driving activity of green fluorescent protein reporter gene in SKBR3, PA1, and T47D cells.

FIG. 21 shows enrichment of a population of cells stably transduced with the ribozyme library cells showing for high expression of EGFP.

FIG. 22 illustrates a reporter plasmid that contains the SV40 promoter driving expression of a bicistronic mRNA containing the coding sequence for hygromycin antibiotic resistance followed by the HCV IRES initiating translation of the HSV thymidine kinase (tk) coding sequence.

FIG. 23 is an illustration of a protocol for identification of genes based on ribozyme sequence tags (rsts).

FIG. 24 provides a schematic diagram of the AMFTdBam construct, which contains a ribozyme under the control of the tRNAval promoter.

FIG. 25 shows the level of extracellular IL-1β production in cultures of THP-1 cells expressing various anti ICE ribozymes.

FIG. 26 shows the reduced production of the CCR-5 tropic strain of HIV (HIV_{BaL}) in PM-1 cultures transduced by anti-CCR-5 ribozymes, but not when the ribozymes are in a catalytically disabled form (indicated by a D suffix). FIG. 26 also shows the confirmation of cell surface expression of CCR-5 by FACS analysis.

FIG. 27 shows puromycin selection on pPur and AMFT transfected A549 and Hela cells.

FIG. 28 illustrates several 5′ and 3′ auxiliary sequences that can be used to enhance ribozyme activity.

FIG. 29 provides time course cleavage reaction data for ribozymes including the stem loop II region of the HIV rev responsive element at the 5′ end along with various lengths of intervening sequence.

FIG. 30 shows the percent recovery of rAAV by batch purification of crude lysate using SP Sepharose High Performance resin.

### DETAILED DESCRIPTION

1. Ribozyme Libraries and Functional Genomics

A principal objective of this invention is to use a “library” of ribozyme genes and/or ribozymes in functional genomic analyses. With the generation of enormous amounts of nucleic acid sequence information by the Human Genome Project, a growing problem has been the assignment of biological activity or function to the identified sequences. This has given rise to the field of functional genomics which is concerned with the assignment of function or activity to nucleic acid sequences (e.g. genomic DNA, mRNA, cDNA, etc.) or to sequences identified by markers (e.g. ESTs, SNPs, etc.).

This invention provides highly efficient methods for identifying nucleic acid sequence previously unknown to be associated with particular phenotypic characters. In a preferred embodiment, the methods of this invention rely on the use of ribozyme libraries (e.g., substantially complete ribozyme libraries) in methods of target acquisition and/or target validation. As used herein, target acquisition refers to the identification and/or isolation of an unknown gene and/or mRNA and /or cDNA whose altered transcription and/or translation produces a detectable change in a phenotypic character. Target acquisition can also refer to the initial (e.g. putative) identification and/or assignment of a function to a previously known gene.

In general terms, methods of target acquisition involve transfecting a cell or population of cells with a ribozyme library (a plurality of ribozymes). One or more biological activities of the cell or population of cells is monitored. Cells showing a change in the monitored activity (i.e., due to transfection with a ribozyme) can be isolated, and the ribozyme or ribozymes contained therein recovered. The ribozymes thus collected can be expanded for subsequent rounds of screening. The binding sites of the ribozymes obtained from the first and/or subsequent rounds of screening can be sequenced. Alternatively, the sequence of the ribozyme binding site(s) can be determined which then provides sequence information suitable for searching nucleic acid databases, for generating probes to probe for the target nucleic acid(s) associated with the alteration of the monitored character, or for use in other applications.

In target acquisition, it is desirable to increase the likelihood of a ribozyme binding to and inhibiting (e.g., cleaving) a nucleic acid (e.g. mRNA) that results in a change in the character (biological activity) that is being monitored. An experiment that utilizes an insufficient diversity of ribozymes (diversity of ribozyme binding sites) has a low or no likelihood of yielding a positive result and thereby costs the research time and money and runs a high risk of never identifying a potentially valuable target. Conversely, the higher the diversity (complexity) of the ribozyme library, the more likely it is to identify a target. In addition, screening with high diversity libraries increases the likelihood that a critical or valuable target will not be missed. Thus, in a particularly preferred embodiment, the methods of this invention, where applicable, are practiced with a complete or substantially complete ribozyme library.

### A) Complete Ribozyme Libraries

As indicated above, to practice the methods of the present invention, it is desirable to produce a library of nucleic acids that encode hairpin ribozymes with randomized or pseudo-randomized recognition sequences. This library is then inserted into a vector of choice for transfecting cells (the particular vector may differ as a function of the application).

It was a discovery of this invention that ribozyme-based functional genomic assays are preferably performed with complete or substantially complete ribozyme libraries with a recognition sequence large enough to not be highly repeated in eukaryotic genomes. Furthermore the target recognition sequence is preferably large enough for the ribozyme to be active.

A complete ribozyme library is one that contains at least one member of every possible binding site having N randomized positions. Thus, for example, where the binding site has one position fully randomized (i.e. the nucleotide at the randomized position can be A, C, G, or T) a complete ribozyme library will contain at least 4 different members (one each having A, G, C, and T at the randomized position). Similarly a complete ribozyme library having two positions fully randomized will contain at least 16 different members. In general, a complete library will contain at least 4ⁿ members where n is the number of positions fully randomized in the binding site.

In generating a random (e.g. complete or substantially complete) ribozyme library, the most critical considerations are 1) the generation of a library with sufficient complexity (number of different members) to assure the presence of ribozymes uniquely specific for any and all given targets, and 2) the competence to package and express, as nearly as possible, the complete library.

However, given current technical capabilities, the synthesis, cloning into viral vectors and efficient delivery into cells of a complex library is not trivial. The more complex the library (i.e., the greater the number of individual ribozyme species), the more difficult it is to clone the complete library into a vector (e.g. plasmid) and then packaged into a viral vector.

As an example, a ribozyme library useful for identifying and targeting a unique gene within the human genome (estimated between 1 to 3×10⁹ base pairs) would require a ribozyme library of sufficient complexity to uniquely recognize any gene in the genome. In order to achieve a suitable degree of binding specificity, the ribozyme sequence tag (RST) recognized by the ribozyme should contain at least about 15 to 16 specific nucleotides. A completely randomized recognition sequence of this size would comprise 4¹⁵1.1×10⁹ to 4¹⁶=4.3×10⁹ different ribozyme species. Due to the inefficiencies of ribozyme-vector ligation, cell transfection, viral vector titer, etc. creating a usable amplifiable (replicatable) library containing 1 to 4×10⁹ different ribozyme molecules and expressing the entire library in a population of transformed cells would be difficult, if not technically impossible prior to the present invention.

It is believed that the desirability and advantages of complete or substantially complete ribozyme libraries were not generally recognized in the art. Thus, previous randomized ribozyme libraries were typically far from complete (see, e.g., U.S. Pat. No. 5,496,698).

It was a discovery of this invention that the hairpin ribozyme is particularly well suited to the production of complete or substantially complete ribozyme libraries. The hairpin ribozyme is unique in its requirement for a GUC or GUA within the target site. Due to this requirement, constructing a library with 15 specific nucleotides (to continue the example described above) requires only 12 random nucleotides, to recognize a substrate in the form: 5′-NNNNXGUCNNNNNNNN-3′ or 5′-NNNNXGUANNN -3′ (the underlined regions indicate basepairs formed with the ribozyme, where N=A,C,G or T and position X has no restrictions and does not interact with the substrate).

Such a hairpin ribozyme library has a complexity of 4¹² (1.7×10⁷) different ribozyme genes or molecules. In comparison, a library of hammerhead ribozymes having a recognition sequence of 15 nucleotides comprises about 10⁹ different species, which have fewer (if any) stringent sequence requirements in the target (Akhtar et al. (1995) Nature Medicine, 1:300; Thompson et al. (1995) Nature Medicine 1:277; Bratty et al. (1993) Biochim. Biophys. Acta., 1216:345; Cech and Uhlenbeck (1994) Nature 372:39; Kijima et al. (1995) Pharmac. Ther., 68:247). In other words, a hammerhead library involving a 15 nucleotide recognition site would require 64 times more individual ribozyme molecules than a hairpin library involving a recognition sequence of equal size. This is a substantial difference.

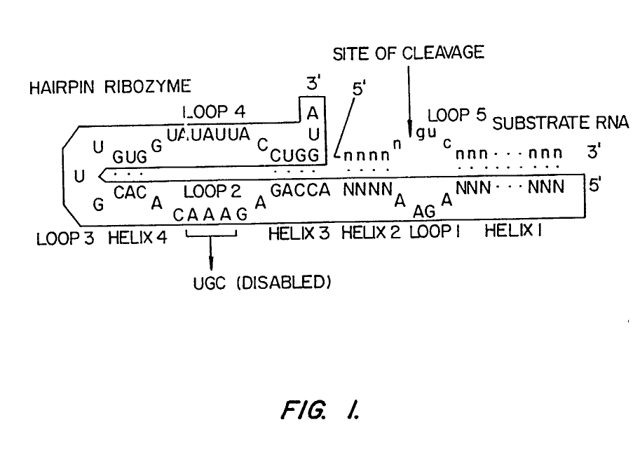

Another advantage that hairpin ribozymes have over hammerhead ribozymes is their intrinsic stability and folding in vivo. The secondary structure of a hammerhead ribozyme, not bound to a target, consists of one helix that is only 4 nucleotides in length which is unlikely to remain intact at physiological temperature, 37° C. (Akhtar et al. (1995) Nature Medicine, 1:300; Thompson et al. (1995) Nature Medicine 1:277; Bratty et al. (1993) Biochim. Biophys. Acta., 1216:345; Cech and Uhlenbeck (1994) Nature 372:39; Kijima et al. (1995) Pharmac. Ther. 68:247). Indeed, the crystal structure of the hammerhead could only be solved when it was bound to a DNA or RNA substrate (Pley et al. (1994) Nature 372:68; Scott et al. (1995) Cell 81:991), suggesting that the hammerhead ribozyme does not have a stable structure prior to substrate binding. In contrast, the hairpin ribozyme contains two helices totaling 7 nucleotides (FIG. 1), thus making it more stable under physiological temperatures and in the intracellular milieu which contains, among other things, RNases that can more effectively cleave RNAs lacking secondary structure. Furthermore, since the hairpin ribozyme has a more stable secondary structure prior to binding substrate, it would be less likely to improperly fold or interact with flanking sequences in the ribozyme RNA transcript. Sequences comprising a hammerhead ribozyme, however, would be free to interact with any extraneous sequences in the transcript resulting in the inactivation of the ribozyme.

Another advantage that hairpin ribozymes have over hammerhead ribozymes is that the cleavage success rate of any given target sequence is higher for the hairpin ribozyme than for the hammerhead. This conclusion has been reached empirically, but can also be explained based on the difference between the two ribozymes' target requirements. The hammerhead ribozyme is very promiscuous, requiring minimal sequence in the target (see above references). Due to its high promiscuity, it has a relatively low success rate when given a variety of potential sites. Conversely, the hairpin ribozyme has significantly more stringent requirements, where its substrate must contain a GUC. Due to the relative rarity of potential sites, the hairpin ribozyme has necessarily developed a higher success rate for cleavage. Indeed, nearly all (>90%) of the potential ribozyme sites we have tested thus far have been cleavable by the appropriate hairpin ribozyme (U.S. applications Ser. Nos. 08/664,094; 08/719,953).

Additionally, one of the applications of the hairpin ribozyme libraries of this invention is the generation of target-specific libraries. One method uses the inherent ability of hairpin ribozymes to catalyze a trans-ligation reaction between cleavage products. This ligation capability is significantly more active in the hairpin ribozyme than in the hammerhead (Berzal-Herranz et al.(1992) Genes and Development 6:1).

Finally, it has been determined empirically that the hairpin ribozyme functions optimally under physiological levels of magnesium (Chowria et al. (1993) Biochemistry 32:1088) and temperature (37° C.), whereas the hammerhead performs optimally at higher magnesium and temperature (Bassi et al. (1996) RNA 2:756; Bennett et al. (1992) Nucleic Acids Research 20:831). These observations become significant when developing and delivering ribozymes in vivo and indicate a clear advantage for hairpin ribozymes.

### B) Substantially Complete Libraries

1) Statistical omissions.

While “complete” ribozyme libraries provide maximal coverage of “sequence space” and provide the greatest likelihood of finding suitable target sites, it is recognized that the creation of a ribozyme gene library and the packaging of such a library is subject to statistical fluctuations that can result in a percentage of ribozymes being under represented or not represented in the library. Nevertheless, because the library is still of sufficiently high complexity (e.g. generally greater than 1×10⁶, more preferably greater than about 1×10⁷, and most preferably greater than about 1×10⁸ or even greater than about 3×10⁸ different members) the likelihood of detecting and knocking down a target is high. Such libraries, while not complete are substantially complete in that they have a substantial number of all possible members. Particularly preferred ribozyme libraries have greater than about 85%, preferably greater than about 90%, more preferably greater than about 95% or even greater than about 98% of all possible hairpin ribozyme binding sequences having seven or more randomized nucleotides. Other preferred substantially complete ribozyme libraries have greater than about 85%, preferably greater than about 90%, more preferably greater than about 95% or even greater than about 98% of all possible hairpin ribozyme binding sequences having eight or more ore even nine or more or even 10 or more or more randomized nucleotides.

Typically substantially complete libraries will have no more than about 1×10¹⁰ members, often no more than about 1×10⁹ different members and occasionally no more than about 1×10⁸ different members.

In addition to the elimination of members due to statistical unpredictabilities, ribozymes may be “eliminated” from substantially complete ribozyme libraries for convenience in storage, or handling or for other considerations.

2) Ribozyme libraries pre-selected to eliminate lethal ribozymes

Transduction with the full ribozyme gene library can result in the expression of ribozymes directed against essential cellular genes. Cells expressing such “toxic” ribozymes will die. This is an especially important consideration when more than one ribozyme is delivered per cell, since the presence of a “toxic” ribozyme would automatically select out any other ribozyme genes in that same cell. In order to minimize the toxicity of the full library, the full library is transduced into the host cells, preferably at an m.o.i. of less than 1, and the ribozyme genes of surviving cells are rescued. The new (e.g., substantially complete) library of rescued ribozyme genes encodes ribozymes that are not fatal to the host cell. This new library can be used to transduce host cells to detect in vivo ribozyme effects, or it can be used to screen for active ribozymes in vitro as described herein. Additionally, this “pre-selection” is a particularly important screening step when it is necessary to introduce multiple ribozyme genes into one cell.

### C) Targeted Ribozyme Libraries

In another embodiment, this invention provides for targeted ribozyme libraries. Targeted libraries contain ribozymes that have been either designed or screened such that the library is enhanced for ribozymes that bind particular pre-selected targets or target families or that are correlated with a particular biological activity or phenotypic character.

Thus, for example, where a particular nucleic acid motif is known, the ribozyme library may be designed to predominantly include ribozymes having binding sites found in the motif. In another embodiment, an initial screening of a complete or substantially complete ribozyme library may identify cells that exhibit ribozyme-induced changes in a particular phenotypic character (i.e., biological activity). The ribozymes may be recovered from these cells and pooled to provide a ribozyme library that is now enhanced (as compared to the original, e.g. substantially complete library) for ribozymes that result in the observed activity or change in activity. Methods of obtaining targeted ribozyme libraries are described in details in the specification and in the examples.

II. Making and Maintaining Libraries of Hairpin Ribozyme-encoding Nucleic Acids Having Randomized Recognition Sequences

The preparation of a hairpin ribozyme library of this invention generally involves the following steps:

a) Provision or creation of a collection of randomized ribozyme inserts;

b) Insertion of randomized genes into “provectors”;

c) Evaluation and verification of ribozyme library complexity.

d) Provision or creation of competent, preferably ultracompetent cells;

e) Transformation of bacteria to expand (amplify) and maintain the ribozyme gene library

f) Recovery and concentration/purification of the vectors (e.g., plasmids) containing ribozyme;

g) Packaging the library into expression vectors that efficiently transfect suitable target cells (e.g. HeLa or A549 cells);

i) Verifying that there is no loss in complexity; and

j) Purifying/concentrating the ribozyme vector library.

To produce a high complexity library (e.g. with >10⁵ different members), greater than one full library must be maintained in order to have statistical confidence that the entire library continues to be represented during each of the steps. This can be calculated using the formula: N=log(1−P)/log[1−(complexity of library)⁻¹], where N is the number of library members actually required and P is the desired probability that all members are present. The practical result is that to produce a high complexity library, each step must preserve a high representation of the library members with relatively low background (vectors that do not encode ribozyme).

Thus, while libraries of relatively low complexity (e.g. less than 10⁵ with a probability of 0.9) can be produced according to standard methods known to those of ordinary skill in the art, the production of high complexity libraries of the present invention required the identification of a number of limitations and problems imposed by prior art methods and the development of novel (non-standard) approaches to overcome these problems. Preferred methods for the production of high complexity ribozyme libraries are described and exemplified herein.

A) Making and Maintaining Libraries of Hairpin Ribozyme-encoding Nucleic Acids Having Randomized Recognition Sequences

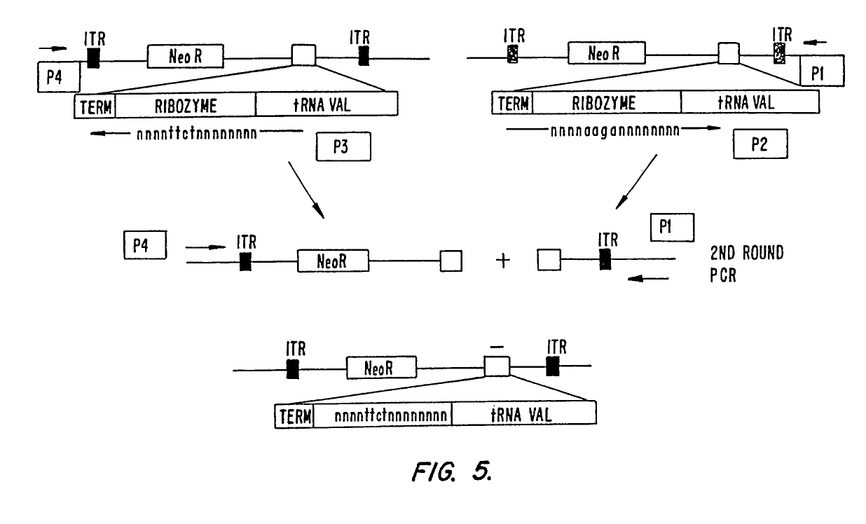

Construction of a library that encodes hairpin ribozyme genes having randomized recognition sequences typically begins with the provision of or creation of a collection of“provectors” encoding ribozymes having randomized recognition sequences (binding sites). The entire ribozyme can be synthesized de novo and then simply ligated into a suitable vector. However, in a preferred embodiment, the random ribozyme libraries are generated in a vector (e.g., pAMFT.dBam and pAGU5 vectors) using multiple rounds of polymerase chain reaction (PCR) with primers of ribozyme sequences containing randomized nucleotides in the substrate binding sites. The protocol is illustrated in FIG. 5 and described in Example 1.

Synthesis of ribozyme-encoding nucleic acids with randomized sequences may be accomplished by any one of a number of methods known to those skilled in the art. See, e.g., Oliphant et al. (1986) Gene 44:177-183; U.S. Pat. Nos. 5,472,840, and 5,270,163. In one approach, the entire ribozyme-encoding nucleic acid is chemically synthesized by known methods one nucleotide at a time, for example in an ABI 380B synthesizer. Whenever it is desired that a given position be randomized, all four nucleotide monomers are added to the reaction mixture; a procedure often referred to in the art as “doping”. After synthesis, the end-products are sequenced by any method known in the art to confirm that the catalytic backbone of the hairpin ribozyme is invariant, and that the recognition sequence is randomized.

In another approach, a randomized oligonucleotide is spliced to the catalytic region of the hairpin ribozyme. This avoids having to chemically synthesize the entire ribozyme.

It should be noted that synthesis and delivery of ribozyme genes rather than RNA ribozymes per se is preferred in the methods of the present invention because: ribozyme genes allow for the constant and continuous production of ribozymes, the ribozyme gene is effectively delivered to the intracellular site of action, and stable gene delivery enables genetic selection of the loss of certain cell functions. The randomized library preferably includes at least 10⁵ ribozyme genes; the upper limit (10⁶, 10⁷, 10⁸, 10⁹ or more) depends on the number of residues in the recognition site.

It was a discovery of this invention that traditional doping methods to produce randomized primers are inadequate for the production of high complexity libraries. Traditional “doping” methods rely on the synthesizer to accurately inject partial amounts (25%) of each oligonucleotide reagent (A, G, C, T, typically as phosphoramidites) in the reaction column during the coupling cycle of the randomized base. Machine-based injection, however, does not provide accurate enough metering to assure uniform representation of all four nucleotides.

Thus, it was discovered that pre-mixing the doping reagent so that a single reagent vial contains all four nucleotide reagents allows the production of adequately uniform “doped” oligonucleotides (see, Example 1).

### B) Insertion of Randomized Ribozyme Genes into a Cloning or Expression Vector

Once the ribozyme library is generated, it is inserted into a cloning or expression vector by methods known in the art, and the library is cloned into suitable cells and amplified. Although cloning and amplification are typically accomplished using bacterial cells, any combination of cloning vector and cell may be used, for low complexity libraries. The cloned cells can be frozen for future amplification and use, or the packaged library can be isolated and itself stored frozen or in lyophilized form.

Typical cloning vectors contain defined cloning sites, origins of replication and selectable genes. Preferably the vector will contain promoter and other elements that will result in optimal activity of the ribozyme so that any single ribozyme will have a high probability of success of gene knockdown in the recipient cells. Expression vectors typically further include transcription and translation initiation sequences, transcription and translation terminators, and promoters useful for regulation of the expression of the particular nucleic acid.

Expression vectors optionally comprise generic expression cassettes containing at least one independent terminator sequence, sequences permitting replication of the cassette in eukaryotes, or prokaryotes, or both, (e.g., shuttle vectors) and selection markers for both prokaryotic and eukaryotic systems. Additionally, the vectors contain a nuclear processing signal, appropriate spicing signals and RNA stability sequences and/or structures (e.g. stable stem-loops, etc.) at either 5′ or 3′ or both ends, all of which will be present in the expressed ribozyme RNA transcript. Vectors are suitable for replication and integration in prokaryotes, eukaryotes, or preferably both.

In a preferred embodiment, the provectors are plasmid provectors. However, it is recognized than numerous other constructs (e.g., cosmid, phagemid, etc.) can be used. For general descriptions of cloning systems and methods, see Giliman and Smith (1979) Gene 8:81-97; Roberts et al. (1987) Nature 328:731-734; Berger and Kimmel (1989) Guide to Molecular Cloning Techniques, Methods in Enzymology, Vol. 152, Academic Press, Inc., San Diego, Calif. (Berger); Sambrook et al. (1989) Molecular Cloning—A Laboratory Manual (2nd ed.) Vols. 1-3, Cold Spring Harbor Laboratory, Cold Spring Harbor Press, N.Y., (Sambrook); and F. M. Ausubel et al. (eds.) (1994) Current Protocols in Molecular Biology, Current Protocols, a joint venture between Greene Publishing Associates, Inc. and John Wiley & Sons, Inc. (1994 Supplement) (Ausubel).

Product information from manufacturers of biological reagents and experimental equipment also provides information useful in known biological methods. Such manufacturers include the SIGMA chemical company (Saint Louis, Mo.), R&D systems (Minneapolis, Minn.), Pharmacia LKB (Piscataway, N.J.), Clontech Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersberg, Md.), Fluka Chemica-Biochemika Analytika (Fluka Chemie A G, Buchs, Switzerland), and Applied Biosystems (Foster City, Calif.), as well as many other commercial sources known to one of skill. Particular expression vectors are discussed in greater detail below.

The nucleic acids (e.g., promoters, vectors, and coding sequences) used in the present method can be isolated from natural sources, obtained from such sources as ATCC or GenBank libraries, or prepared by synthetic methods. Synthetic nucleic acids can be prepared by a variety of solution or solid phase methods. Detailed descriptions of the procedures for solid phase synthesis of nucleic acids by phosphite-triester, phosphotriester, and H-phosphonate chemistries are widely available. See, for example, Itakura, U.S. Pat. No. 4,401,796; Caruthers, et al., U.S. Pat. Nos. 4,458,066 and 4,500,707; Beaucage, et al. (1981) Tetra. Lett. 22:1859-1862; Matteucci et al. (1981) J. Am. Chem. Soc. 103:3185-3191; Caruthers, et al. (1982) Genetic Eng. 4:1-17; Gait (ed.) (1984) Oligonucleotide Synthesis: A Practical Approach, IRL Press, Washington D.C.; Froehler, et al. (1986) Tetrahedron Lett. 27:469-472; Froehler et al. (1986) Nucleic Acids Res. 14:5399-5407; Sinha, et al. (1983) Tetrahedron Lett. 24:5843-5846; and Sinha, et al. (1984) Nucl. Acids Res. 12:4539-4557.

In the production of high complexity libraries, the ribozyme nucleic acids are preferably PCR cloned into the vector. Thus, as illustrated in a preferred embodiment, the random ribozyme libraries are generated in a vector (e.g., pAMFT.dBam and pAGU5 vectors) using multiple rounds of polymerase chain reaction (PCR) with primers of ribozyme sequences containing randomized nucleotides in the substrate binding sites. The protocol is illustrated in FIG. 5 and described in Example 1.

It was a discovery of this invention that the production of high complexity libraries required a low background (vectors having no inserts). Therefore, the vectors were designed to include a 2 kb insert, e.g., between the ITRs in the AAV vector. The insert allows vectors containing the ribozyme insert to be separated (e.g. electrophoretically) from the vectors lacking the ribozyme insert. It will be recognized that much small inserts allow the separation, however, the vectors e.g., AAV cannot package the larger nucleic acid and so the large size insert also prevents background by prohibiting packaging of non-ligated (with ribozyme insert) vectors.

In addition, rather than using a kinase to phosphorylate the oligonucleotides prior to ligation the oligonucleotides were chemically synthesized with a terminal phosphate. It was discovered that chemical addition of the 5′ phosphate is much more efficient and more easily controlled than enzymatic addition using T4 polynucleotide kinase.

Finally, it was discovered that production of high complexity libraries was enhanced by using at least an 8-fold molar excess of insert to vector. It was a discovery of this invention that less insert:vector caused vector to reclose without any insert (as measured by the destruction of both restriction sites), thus increasing the background of empty vector. This phenomenon was due to our extremely high ligation and transformation efficiencies.

### C) Evaluation and Verification of Ribozyme Library Complexity

The “complexity” of the ribozyme library, or the total number of unique members, is dependent on the number of randomized bases in the ribozyme binding arms. A fully complex ribozyme library consisting of eight randomized bases in helix 1 and four randomized bases in helix 2 (for a total of 12 randomized bases) would contain 4¹² (or 1.68×10⁷) different members.

When actually working with and manipulating a library such as this, however, greater than one full library must be maintained in order to have statistical confidence that the entire library continues to be represented. This can be calculated using the formula: N=log(1−P)/log[1−(complexity of library)⁻¹], where N is the number of library members actually required and P is the desired probability that all members are present (Moore (1987) Current Protocols in Molecular Biology). To continue the example above, to have 95% confidence that all members are present in a library with 12 randomized bases, 5.03×10⁷ ribozymes are necessary and therefore 5.03×10⁷ bacterial plasmid transformants to generate a renewable library. Similarly, 99% confidence requires 7.73×10⁷ total ribozymes.

Ribozyme library complexity is verified both qualitatively and quantitatively. The first involves in vitro transcribing the entire ribozyme library in one reaction and then evaluating its ability to cleave a variety of different RNA substrates, of both cellular and viral origin. In addition, the ribozyme library DNA can be subjected to DNA sequencing and a properly prepared library will result in equal band intensity across all four sequencing lanes for each randomized position.

The second method involves statistical analyses of individual ribozymes (picked from the library of bacterial transformants and sequenced) to build confidence intervals for each base position in each molecule, thus allowing an evaluation of the complexity of the library without having to manually sequence each individual ribozyme. The formula for a two-sided approximate binomial confidence interval is E=1.96*squareroot(P*(1−P)/N), where P is the expected proportion of each nucleotide in a given position (which for DNA bases equals 25% or P=0.25), E is the desired confidence interval around P (i.e. P±E) and N is the required sample size (Callahan Associates Inc., La Jolla, Calif.). For example, if we need to know the proportion of each base within 5% (E=0.05), then the required sample size is 289. Since each ribozyme molecule contains twelve independent positions, the number of ribozymes that need to be sequenced out of the pool equals 289÷12, or about 25 molecules.

### D) Provision or Creation of Competent, Preferably Ultracompetent Cells

The expansion (amplification) and maintenance of a ribozyme library can be accomplished in virtually any cell routinely used for maintenance of plasmids and/or viral vectors. Of course, the cell should be selected that is compatible with the vector.

Suitable cells include, but are not limited to a wide variety of bacterial cells including, but not limited to, E. coli, Bacillus subtilis, Salmonella, Serratia, and various Pseudomonas species. Generation of a sufficiently complex ribozyme plasmid library requires bacteria of extremely high competency. Bacterial electroporation typically yields the highest transformation efficiency so high competency electrocompetent cells are preferred. Example 5 describes the production of electrocompetent cells from the strain DH12S.

These electrocompetent cells must be extremely competent in order to generate a library of sufficient complexity. The cells are electroporated with a Bio-Rad Gene Pulser® II with a capacitance of 25 μF and a resistance of 200 ohms. The competency level of the cells is always tested by transforming them with a supercoiled plasmid and at least 1×10¹⁰ transformants per μg of DNA must be obtained for the cells to be used for library transformations, because the ligated ribozyme library will not transform as efficiently as supercoiled DNA. To be sure we had the most highly competent cells possible, we compared our cells head to head with ElectroMAX DH12S™ cells from Gibco/BRL. Our cells consistently gave more transformants when identical transformation conditions were carried out.

### E) Transformation of Bacteria to Expand (Amplify) and Maintain the Library

1) Transformation of cells

The nucleic acid constructs encoding the substantially complete population of ribozymes can be use to transform bacteria to expand (amplify) and/or maintain the ribozyme gene library. Transformation of bacterial cells is by standard methods well known to those of skill in the art. There are several well-known methods of introducing nucleic acids into bacterial, animal or plant cells, any of which may be used in the present invention. These include: calcium phosphate precipitation, fusion of the recipient cells with bacterial protoplasts containing the nucleic acid, treatment of the recipient cells with liposomes containing the nucleic acid, DEAE dextran, receptor-mediated endocytosis, electroporation, micro-injection of the nucleic acid directly into the cells, infection with viral vectors, etc. Cationic liposomes-mediated delivery of AAV-ribozyme-library pro-vector plasmid may be employed (Philp et al. (1994) Mol. Cell. Biol. 14:2411-2418).

In one preferred embodiment, electroporation as described above and in the examples performed.

2) Host cells and culture.

E. coli is one prokaryotic host useful for maintaining and/or expanding the DNA sequences of the present invention. Other microbial hosts suitable for use include bacilli, such as Bacillus subtilis, and other Enterobacteriaceae, such as Salmonella, Serratia, and various Pseudomonas species. Other suitable prokaryotic hosts are well known to those of skill in the art, see, e.g., Sambrook et al. (1989) supra. or Ausubel et al. (ed.) (1987) supra.

The transformed cells can be maintained and/or expanded using standard bacterial culture methods well known to those of skill in the art (see, e.g. the Examples and Sambrook et al. (1989) supra. or Ausubel et al. (ed.) (1987) supra.).

3) Recovery of the ribozyme gene library.

The ribozyme gene library can be recovered according to standard methods well known to those of skill in the art. Standard methods for recovery of plasmids (or other constructs) from bacterial hosts are well known to those of skill in the art (see, e.g. the Examples and Sambrook et al. (1989) supra. or Ausubel et al. (ed.) (1987) supra.).

4) Vectors useful for maximal ribozyme expression

The vector comprising the expression cassette encoding the ribozyme will be selected so as to be compatible with maintenance of a ribozyme library in cell culture and so as to provide effective transfection of target cells in vitro and in vivo in the target acquisition and target validation methods of this invention. A number of viral vector systems can be used to express ribozyme libraries in vivo, including retroviral vectors, vaccinia vectors, herpes simplex vectors, Sindbis/semliki forest viruses, adenoviral vectors, and adeno-associated viral (AAV) vectors. Each vector system has advantages and disadvantages, which relate to host cell range, intracellular location, level and duration of transgene expression and ease of scale-up/purification. Optimal delivery systems are characterized by: 1) broad host range; 2) high titer/μg DNA; 3) stable expression; 4) non-toxic to host cells; 5) no replication in host cells; 6) ideally no viral gene expression; 7) stable transmission to daughter cells; 8) high rescue yield; and 9) lack of subsequent replication-competent virus that may interfere with subsequent analysis. Choice of vector may depend on the intended application.

### (a) AAV Vectors

Because of their demonstrated ease of use, broad host range, stable transmission to daughter cells, high titer/pg DNA, and stable expression, (Lebkowski et al. (1988) Mol. Cell. Biol. 8:3988-3996), adeno-associated viral vector are preferred to deliver ribozyme library genes into target cells. See, e.g., Goeddel (ed.) (1990) Methods in Enzymology, Vol. 185, Academic Press, Inc., San Diego, Calif. or M. Krieger (1990) Gene Transfer and Expression—A Laboratory Manual, Stockton Press, New York, N.Y., and the references cited therein. AAV requires helper viruses such as adenovirus or herpes virus to achieve productive infection.

AAV displays a very broad range of hosts including chicken, rodent, monkey and human cells (Muzycka (1992) Curr. Top. Microbiol. Immunol. 158, 97-129; Tratschin et al. (1985) Mol. Cell. Biol. 5: 3251-3260; Lebkowski et al. (1988) Mol. Cell. Biol. 8: 3988-3996. They efficiently transduce a wide variety of dividing and non-dividing cell types in vitro (Flotte et al. (1992) Am. J. Respir. Cell. Mol. Biol. 7, 349-356; Podsakoff et al. (1994) J. Virol. 68: 5655-5666, Alexander et al. (1994) J. Virol, 68: 8282-8287). AAV vectors have been demonstrated to successfully transduce hematopoietic progenitor cells of rodent or human origin (Nahreini et al. (1991) Blood, 78:2079). It is believed that AAV could virtually infect any mammalian cell type.

Moreover, the copy number for the neo gene introduced by the AAV vector is more than 2 orders of magnitude higher than that of retrovirally-transduced human tumor-infiltrating lymphocyte (TIL) cell cultures. Long-term in vivo gene expression has recently been demonstrated in the lungs of rabbit and primates that received AAV-CFTR vector in a local pulmonary administered for up to six months (Conrad et al. (1996) Gene Therapy 3: 658-668). Administration of the AAV-CFTR gene product resulted in consistent gene transfer, and persistence of the gene in one human parent out to 70 days (10th Annual North American Cystic Fibrosis Conference, Orlando, Fla., Oct. 25-27, 1996).

Integration is important for stable transgene expression, especially in cells that are actively dividing. Site-specific integration is even better since there is less chance of disrupting a cellular gene, less chance of inactivating the target gene by the insertion and it lends itself to more consistent expression of the delivered transgene. In the absence of helper virus functions, AAV integrates (site-specifically) into a host cell's genome. The integrated AAV genome has no pathogenic effect. The integration step allows the AAV genome to remain genetically intact until the host is exposed to the appropriate environmental conditions (e.g., a lytic helper virus), whereupon it re-enters the lytic life-cycle. Samulski (1993) Current Opinion in Genetic and Development 3:74-80, and the references cited therein provides an overview of the AAV life cycle. See also West et al. (1987) Virology 160:38-47; Carter et al. (1989) U.S. Pat. No. 4,797,368; Carter et al. (1993) WO 93/24641; Kotin (1994) Human Gene Therapy 5:793-801; Muzyczka (1994) J. Clin. Invest. 94:1351 and Samulski, supra, for an overview of AAV vectors.

Although wild-type AAV reportedly integrates efficiently at a specific site on chromosome 19 (Kotin, et al. (1990) Proc Natl Acad Sci USA 87 2211-2215; Kotin et al. (1992) EMBO J 11:5071-5078; Samulski et al. (1991) EMBO J, 10: 3941-3950; Samulski (1993) Curr Opin Biotech, 3: 74-80) recent evidence indicates that rep-deleted AAV vectors do not integrate with any appreciable efficiency or specificity. Flotte et al. (1994) Am J. Resp Cell Mol Biol 11: 517-521; Kearns et al. (1996) Gene Therapy 3:748; Fisher-Adams et al. (1996) Blood 88:492). Data generated using Southern and fluorescent in situ hybridization (FISH) analyses, indicates that rAAV integrates into a finite number of chromosomal sites, possibly hot spots for recombination.

Once a cell or cells have been selected and shown to contain the ribozyme(s) of interest, the entire AAV-ribozyme expression cassette can be easily “rescued” from the host cell genome and amplified by introduction of the AAV viral proteins and wild type adenovirus (Hermonat. and Muzyczka (1984) PNAS. USA 81:6466-6470; Tratschin. et al. (1985) Mol. Cell. Biol. 5:3251-3260; Samulski et al. (1982) PNAS USA 79:2077-2081; Tratschin et al. (1985) Mol. Cell. Biol. 5:3251-3260). This makes isolation, purification and identification of selected ribozymes considerably easier than other molecular biology techniques.

### (b) Retroviral Vectors

Retroviral vectors may also be used in certain applications. The design of retroviral vectors is well known to one of skill in the art. See Singer, M. and Berg, P., supra. In brief, if the sequences necessary for encapsidation (or packaging of retroviral RNA into infectious virions) are missing from the viral genome, the result is a cis acting defect which prevents encapsidation of genomic RNA. However, the resulting mutant is still capable of directing the synthesis of all virion proteins. Retroviral genomes from which these sequences have been deleted, as well as cell lines containing the mutant genome stably integrated into the chromosome are well known in the art and are used to construct retroviral vectors. Preparation of retroviral vectors and their uses are described in many publications including European Patent Application EPA 0 178 220, U.S. Pat No. 4,405,712; Gilboa (1986) Biotechniques 4:504-512, Mann et al. (1983) Cell 33:153-159; Cone and Mulligan (1984) Proc. Natl. Acad. Sci. USA 81:6349-6353, Eglitis et al. (19188) Biotechniques 6:608-614; Miller et al. (1989) Biotechniques 7:981-990; Miller (1992) Nature, supra; Mulligan (1993) supra; and Gould et al., and International Patent Application No. WO 92/07943 entitled “Retroviral Vectors Useful in Gene Therapy.” The teachings of these patents and publications are incorporated herein by reference.

The retroviral vector particles are prepared by recombinantly inserting a nucleic acid encoding a nucleic acid of interest into a retrovirus vector and packaging the vector with retroviral capsid proteins by use of a packaging cell line. The resultant retroviral vector particle is generally incapable of replication in the host cell and is capable of integrating into the host cell genome as a proviral sequence containing the calbindin nucleic acid. As a result, the host cell produces the gene product encoded by the nucleic acid of interest.

Packaging cell lines are generally used to prepare the retroviral vector particles. A packaging cell line is a genetically constructed mammalian tissue culture cell line that produces the necessary viral structural proteins required for packaging, but which is incapable of producing infectious virions. Retroviral vectors, on the other hand, lack the structural genes but have the nucleic acid sequences necessary for packaging. To prepare a packaging cell line, an infectious clone of a desired retrovirus, in which the packaging site has been deleted, is constructed. Cells comprising this construct will express all structural proteins but the introduced DNA will be incapable of being packaged. Alternatively, packaging cell lines can be produced by transducing a cell line with one or more expression plasmids encoding the appropriate core and envelope proteins. In these cells, the gag, pol, and env genes can be derived from the same or different retroviruses.

A number of packaging cell lines suitable for the present invention are available in the prior art. Examples of these cell lines include Crip, GPE86, PA317 and PG13. See Miller et al. (1991) J. Virol. 65:2220-2224, which is incorporated herein by reference. Examples of other packaging cell lines are described in Cone and Mulligan (1984) Proceedings of the National Academy of Sciences, U.S.A. 81:6349-6353 and in Danos and Mulligan (1988) Proceedings of the National Academy of Sciences, U.S.A. 85:6460-6464; Eglitis et al. (1988) Biotechniques 6:608-614; Miller et al. (1989) Biotechniques 7:981-990, also all incorporated herein by reference. Amphotropic or xenotropic envelope proteins, such as those produced by PA317 and GPX packaging cell lines may also be used to package the retroviral vectors.

Although retroviral vectors (RVV) have been used extensively in the past, and could be used to deliver our ribozyme gene library, they are not the most preferred vector for several reasons: 1) it is difficult to produce and purify FVV to high titer, 2) the virus is enveloped and therefore is relatively unstable during storage or freeze/thaw, 3) RVV genomes are positive strand RNA, which would be a target for ribozymes in the library and 4) while they do stably integrate into the host genome, the integration step requires one round of cell division, which could be problematic when delivering is in vivo or to non-dividing cells.

### (c) Sindbis/Semliki Forest Viruses

Sindbis/semliki forest viruses (Berglund et al. (1993) Biotechnology 11:916-920) are positive-strand RNA viruses that replicate in the cytoplasm, are stably maintained, and can yield very high levels of antisense RNA. Sindbis vectors are thus a third type of vector useful for maximal utility.

4) Promoters useful for ribozyme expression

The promoters used to control the gene expression from AAV include: (a) viral promoters such as SV40, CMV, retroviral LTRs, herpes virus TK promoter, parvovirus B-19 promoter (Muzycka, N, 1992, Curr. Top. Microbiol. Immunol. 158, 97-129), AAV p5 and p40 promoters (Tratschin et al., 1993. Am. J. Respir. Cell. Mol. Biol. 7, 349-356). (b) human gene promoters such as the gamma-globin promoter (Walsh et al., 1992, Proc. Nat. Acad. Sci., USA 89, 7257-7261), the β-actin promoter, or integrin CD11a or CD11b; and (c) RNA pol III promoters such as cellular tRNA promoters or the promoter from the adenovirus VA1 gene (U.S. application Ser. No. 08/664,094; U.S. application Ser. No. 08/719,953). Particularly preferred promoters are the tRNA promoters including, but not limited to the tRNA valine promoter (tRNAval) and the tRNAserine promoter (tRNAser), as well as the cellular house-keeping promoter, phosphoglycerate kinase (PGK).

5) 5′ and 3′ auxiliary sequences

In preferred embodiments, auxiliary sequences are added to the 5′ or 3′ termini of a ribozyme. Such auxiliary sequences enhance the activity of the ribozymes. For example, the stem loop II region of the HIV rev responsive element can be added to the 5′ end of the ribozyme, preferably with an intervening sequence, e.g., 10, 30, 50, 70, 100, or more nucleotides of intervening sequence. Particularly preferred is the addition of about 50 bases of intervening sequence. In certain embodiments, additional sequences will be added to the 3′ end of a ribozyme, thereby enhancing the activity of the ribozyme. For example, a tetraloop RNA sequence can be added, preferably with an intervening spacer sequence, e.g., a 6 base intervening sequence. Such embodiments can also comprise a substrate sequence, whereby the ribozyme is an autocatalytic ribozyme, which can efficiently cleave at the substrate sequence. Such self-cleaved ribozyme molecules, e.g., with an 8 base spacer between the tetraloop and the substrate sequence, are at least as active as the unmodified ribozyme.

### G) Packaging the Library into Expression Vectors that Efficiently Transfect Suitable Target Cells

Packaging of the vectors comprising the ribozyme gene library is accomplished according to standard methods well known to those of skill in the art. Many vectors (e.g., EBV, retrovirus vectors, etc., are capable of self-packaging. However, a number of viral vectors (e.g. AAV) typically require helper virus (e.g. adenovirus, or herpes virus) or cells containing the necessary “machinery” to facilitate packaging; so called helper cells.

In a preferred embodiment, the cells (e.g. helper cells) are transfected with ribozyme gene constructs. Helper cells will contain the ancillary “machinery” to facilitate packaging of the construct into a virion. Alternatively cells are co-transfected with the ribozyme vector and a helper virus (e.g. adenovirus to help AAV) to facilitate.

### I) Verifying that There is No Loss in Complexity

Particularly when maintaining high-complexity libraries, it is desirable that there be no or little loss in complexity in packaging the ribozyme library. The library complexity can be monitored according to any of a number of ways. In one preferred embodiment, the complexity of the ribozyme library is monitored as follows:

Cells expressing an HSV-tk gene or transduced with an pHSV-TK gene are transduced with either an AAV vector or an AAV-ribozyme-Lib vector, and cultured in the presence of gancyclovir and G418. Cells that lack a functional ribozyme that cleaves the tk mRNA will express thymidine kinase and die. Cells that inactivate the HSV tk gene product with one or more specific ribozymes will survive. Surviving cells are amplified, and the sequence of the anti-HSV tk ribozyme is determined by PCR of ribozyme gene(s), followed by sequencing analysis of the amplified product. The ribozyme gene sequences that are complementary to regions of the tk gene sequence can be used as a gene probe for HSV tk gene. Once ribozymes that appear to inactive tk have been isolated, their catalytic activity can be verified by converting them into “disabled” ribozymes (i.e. disrupting their catalytic activity without affecting substrate binding, see section 2.h. How to distinguish between ribozyme effects . . . above for a more detailed description) followed by re-analyzing their effects in vivo.

Alternatively, cells expressing any other selectable or FACS-sortable marker, such as green fluorescent protein (GFP) or Erb, can also be used as the target for testing the complexity of the invented AAV-ribozyme library vector.

### J) Purifying/Concentrating the Ribozyme Vector Library

AAV particle generation by transient transfection is optimized to yield the highest possible AAV titer with a minimum amount of DNA. This step is crucial for assuring a vector gene library with maximal sequence complexity. Once all the procedures have been optimized, ribozyme gene vector libraries are generated by transient transfection on AAV packaging cell lines and purified by column chromatography. Column purification is carried out only if necessary for optimal transduction efficiency and depending on the desired application. Vector is then applied to a given cell and the desired phenotype is analyzed. Ribozyme sequences in the transduced cells are identified, amplified and rescued with wild type AAV or helper plasmids and helper virus (such as adenovirus). The rescued vector is then used again to transduce the target cells and the cycle repeated. AAV and adenovirus can be selectively inactivated or purified. Any remaining wild type AAV will be inert since it cannot replicate without a helper virus.

Until now, use of AAV as a useful gene delivery vehicle has been hampered by the inability to produce high titer virus (Hermonat and Muzyczka (1984) PNAS USA 81:6466; Samulski et al. (1987) J. Virol. 61:3096). Indeed, the typical yield of rAAV vectors currently reported in the literature is approximately 105 colony-forming units/ml (Kaplitt et al. (1994) Nature Genetics 8:148; Miller et al.(1994) PNAS USA 91:10183; Samulski et al. (1989) J. Virol. 63:3822).

Now, however, proprietary production and purification methods developed at Immusol yield high titers (greater than 5×10⁸ infectious particles/ml) with no wild type helper virus contamination. This is in stark contrast to published data (see references above). High viral titers are extremely important for constructing complete ribozyme libraries; for performing efficient, high m.o.i. transductions as well as making feasible any in vivo (animal) applications of the library or selected libraries.

Immusol, Inc. has previously developed the technology of “increased titer of recombinant AAV vectors by gene transfer with adenovirus coupled to DNA polylysine complexes”. This method was published in Gene Therapy (vol.2, pp429, 1995). This technology is licensed to Immusol and has been used as our routine rAAV preparations for all pre-clinical studies. Recently this technique has been adapted to large-scale preparation of purified rAAV at high titer using CsCl₂ centrifugation.

Lysing the producer cells with the non-ionic detergent octylglucoside or the ionic detergent deoxycholate appears to increase the titer substantially compared with the freeze-thaw procedure used previously to extract the AAV particles from the cells. Octylglucoside may be of further advantage since it will allow for direct loading of material onto ion-exchange columns if desired (FPLC).

After carefully testing the rAAV titer in the CFU system, we concluded that we can reproducibly obtain high titer purified rAAV. Peak titers are in excess of 5×10⁸/ml (neo colony forming units, CFU). The total yield from a single prep is more than 5×10⁹ CFU at an average titer of 1×10⁸ CFU/ml

High titer retrovirus is obtainable by pseudotyping the retrovirus as described in the examples.

In certain embodiments, rAAV vectors can be partially purified from crude cell lysate preparations using rapid purification chromatographic methods, e.g., SP sepharose High Performance resin (Pharmacia) and/or POROS 50HQ resin (Perceptive Biosystems)

III. Uses of Ribozyme Libraries in Target Acquisition

As described above, hairpin ribozyme libraries with randomized ribozyme recognition sites are used in a variety of Ribozyme-Mediated Gene Functional Analyses (RiMGFA), in which comparison of biological properties of cells with or without gene-inactivating ribozymes reveals the function and/or identity of a given gene. Generally speaking, the methods can be classified as target acquisition methods. However it will be appreciated that selection methods utilizing substantially complete ribozyme libraries of this invention can also be used in a variety of other methods including, but not limited to, the generation of target specific ribozymes, or target-specific ribozyme libraries.

The target acquisition methods generally entail:

A) Transfecting a cell or population of cells with a ribozyme library, preferably a complete or substantially complete ribozyme library of high complexity.

B) One or more biological activities of the cell or population of cells is monitored.

C) Cells showing a change in the monitored activity (i.e., due to transfection with a ribozyme) can be isolated;

D) The ribozyme or ribozymes contained in the cells are recovered.

E) The collected ribozymes are optionally expanded for subsequent rounds of screening;

F) The binding sites of the ribozymes obtained from the first and/or subsequent rounds of screening are optionally sequenced.

G) Optionally the sequence information is used to search sequence databanks (e.g. GenBank) or to design probes to specifically identify and/or isolate the target(s) to which the ribosome(s) bound.

As indicated above, the use of substantially complete ribozyme libraries of high complexity increases the likelihood of target identification and diminishes the likelihood of missed critical targets. In addition, the use of stably transfected ribozymes allows screening of phenotypic characters that might either be suppressed by transient transfection methods or that may take several generations of cell replication to fully or detectably manifest.

### A) Transfecting a Cell or Population of Cells with a Ribozyme Library, Preferably a Complete or Substantially Complete Ribozyme Library of High Complexity

1) Cell or cell population transfection

In methods of target acquisition, a cell, more preferably a population of cells is transfected with a hairpin ribozyme vector library. The cells or population of cells can comprise individual cells in culture (e.g., in adherent layers or in solution), cells in tissues, cells as components of organs, organ systems, and even in vivo in entire organisms. For in vitro applications, the delivery of ribozyme library members can be to any cell that can be grown or maintained in culture, whether of bacterial, plant or animal origin, vertebrate or invertebrate, and of any tissue or type. Although any prokaryotic or eukaryotic cells may be used, the preferred cell will be one in which the target gene is normally expressed (i.e. liver cells for liver-specific genes, tumor cells for oncogenes, etc.) or has been caused to be expressed. Furthermore, the cell would preferably contain a reporter or sortable gene to expedite the selection process.

Transfection of the cells is according to standard methods known to those of skill in the art. Particularly, for in vivo applications, in a preferred embodiment, the viral vectors (e.g. retroviruses, AAV, EBV, HIV, etc.) themselves are competent to transfect the cells, although it will be recognized that the cells can also be transfected in vivo using other systems (e.g. by lipid- or liposome-mediated transfection systems).

Where the cells are cultured in vitro additional transfection methods (e.g. electroporation, lipid-mediated transection, etc.) are available.

Contact between the cells and the genetically engineered nucleic acid constructs or viral particles, when carried out in vitro, takes place in a biologically compatible medium. The concentration of nucleic acid varies or viral particle widely depending on the particular application. Nucleic acid concentrations are generally between about 1 micromolar and about 10 millimolar. Treatment of the cells with the nucleic acid is generally carried out at physiological temperatures (37° C.) for periods of time of from about 1 to 48 hours, preferably of from 4 to 12 hours.

For viral transduction, cells are incubated with vector at an appropriate multiplicity of infection (m.o.i.)(depends on application, see below) for 4 to 16 hours (Flotte et al. (1994) Am. J. Resp. Cell Mol. Biol. 11:517).

In one group of embodiments, a nucleic acid is added to 60-80% confluent plated cells having a cell density of from about 10³ to about 10⁵ cells/mL, more preferably about 2×10⁴ cells/mL. The concentration of the suspension added to the cells is preferably of from about 0.01 to 0.2 micrograms/mL, more preferably about 0.1 micrograms/mL.

2) Maintenance of cell lines.

The cells can be maintained according to standard methods well known to those of skill in the art (see, e.g., Freshney (1994) Culture of Animal Cells, A Manual of Basic Technique, (3d ed.) Wiley-Liss, New York; Kuchler et al. (1977) Biochemical Methods in Cell Culture and Virology, Kuchler, R. J., Dowden, Hutchinson and Ross, Inc. and the references cited therein). Cultured cell systems often will be in the form of monolayers of cells, although cell suspensions are also used.

In a preferred embodiment, one or more reporter genes are used to identify those cells that are successfully transfected. The same or a different reporter gene can be expressed by the expression cassette expressing the ribozyme to provide an indication of actual ribozyme expression.

A reporter gene (also, marker gene) is one whose gene product is readily inducible and/or detectable, that is used to detect cells that are transduced with a vector that encodes the reporter gene, to isolate and clone such cells, and to monitor the effects of environmental and cytoplasmic factors on gene expression in the transduced cells. Preferred reporter genes are those that render cells FACS-sortable: e.g., genes for fluorescent proteins, including green fluorescent protein (GFP) and any mutant thereof, nerve growth factor receptor (NGFR) and any mutant thereof; genes for cell surface proteins that may be coupled to easily detected ligands such as fluorescent antibodies. Specific reporter genes that can be selected for or against in tissue culture, which may be used herein include the hprt gene (Littlefield (1964) Science 145:709-710), the tk (thymidine kinase) gene of herpes simplex virus (Giphart-Gassler et al. (1989) Mutat. Res. 214:223-232), the nDtII gene (Thomas et al. (1987) Cell 51:503-512; Mansour et al. (1988) Nature 336:348-352), or other genes which confer resistance or sensitivity to amino acid or nucleoside analogues, or antibiotics, etc.

For the most part, reporter genes are used herein to identify cells that have been transduced with nucleic acids that encode a ribozyme and or a gene of interest. It is possible that a given cell clone identified as under-expressing the reporter gene may contain a ribozyme gene that cleaves the gene product of the reporter gene instead of the gene of interest, in which case the ribozyme genes against the reporter gene will be mis-identified as ribozymes directed against the gene of interest. Thus, it is preferable to generate a cell line that co-expresses at least two or three different reporter genes linked to the gene of interest. Only ribozyme genes that inhibit the gene of interest will result in under-expression of more than one reporter gene simultaneously. Alternatively, it may be necessary to pre-screen the library to ensure that the reporter RNA is not the target of the ribozyme attack. In addition, pre-screening may also be required to ensure that the presence of any reporter RNA does not alter accessibility or structure of the target RNA.

2) Ribozyme expression in transgenic and chimeric animals

The ribozymes in the ribozyme library can also be expressed in a chimeric animal or in a non-human transgenic animal. The transgenic animals of the invention comprise any non-human animal or mammal, such as non-human primates, ovine, canine, bovine, rat and murine species as well as rabbit and the like. Preferred non-human animals are selected from the rodent family, including rat, guinea pig and mouse, most preferably mouse.

Generally, a female non-human animal is induced to superovulate by the administration of hormones such as follicle-stimulating hormone, the eggs are either collected and fertilized in vitro or the superovulated female is mated to a male and the zygotes are collected, and the zygote is transduced with one or more selected vectors comprising a ribozyme library and/or a preselected nucleic acid. In the case of zygotes the preferred method of transgene introduction is by microinjection. However, other methods such as retroviral or adenoviral infection, electroporation, or liposomal fusion can be used.

Specific methods for making transgenic non-human animals are described in the following references: Pinkert C. A. (ed.) (1994) Transgenic Animal Technology: A Laboratory Handbook Academic Press and references cited therein; Pursel et al. (1989) Genetic engineering of livestock, Science 244:1281-1288, especially p. 1282-1283, Table 1 at p. 1283; Elbrecht A. et al. (1987) “Episomal Maintenance of a Bovine Papilloma Virus Vector in Transgenic Mice,” Mol. Cell. Biol. 7(3):1276-1279; Hammer et al. (1985) “Production of transgenic rabbits, sheep and pigs by microinjection,” Nature 315:680-683; Hughes et al. (1990) “Vectors and genes for the improvement of animal strains” J. Reprod. Fert., Suppl. 41:39-49; Inoue et al. (1989) “Stage-dependent expression of the chicken α-crystallin gene in transgenic fish embryos,” Cell Differen. Devel. 27:57-68; Massey (1990) J. Reprod. Fert., Suppl., 41:199-208; Rexroad, C., et al. (1989) Mol. Reprod. Devel. 1:164-169; Rexroad et al. (1990), J. Reprod. Fert., Suppl., 41:119-124; Simons et al. (1988) Bio/Technology, 6:179-183; Squire et al. (1989) Am. J. Vet. Res., 50(8) 1423-1427; WallJ. (1989) Animal Genetics, 20:325-327; Ward et al. (1990) Rev. Sci. Tech. Off. Int. Epiz., 9(3):847-864; Westphal (1989) FASEB J., 3:117-120.

In the mouse, the male pronucleus reaches the size of approximately 20 micrometers in diameter which allows reproducible injection of 1-2 pl of DNA solution. The use of zygotes as a target for gene transfer has a major advantage in that in most cases the injected DNA will be incorporated into the host gene before the first cleavage (Brinster, et al. (1985) Proc. Natl. Acad. Sci. USA 82:4438-4442). As a consequence, all cells of the transgenic non-human animal will carry the incorporated transgene. This will, in general, also be reflected in the efficient transmission of the transgene to offspring of the founder since 50% of the germ cells will harbor the transgene.

The gene sequence being introduced need not be incorporated into any kind of self-replicating plasmid or virus (Jaenisch (1988) Science 240:1468-1474 (1988)). Indeed, the presence of vector DNA has been found, in some cases, to be undesirable (Hammer et al. (1987) Science 235:53; Chada et al. (1986) Nature 319:685; Kollias et al. (1986) Cell 46:89; Shani (1986) Molec. Cell. Biol. 6:2624 (1986); Chada et al. (1985) Nature, 314:377; Townes et al. (1985) EMBO J. 4:1715).

Once members of a ribozyme library, or any other DNA molecule are injected into the fertilized egg cell, the cell is implanted into the uterus of a receptive female (i.e., a female whose uterus is primed for implantation, either naturally or by the administration of hormones), and allowed to develop into an animal. Since all of the animal's cells are derived from the implanted fertilized egg, all of the cells of the resulting animal (including the germ line cells) shall contain the introduced gene sequence. If, as occurs in about 30% of events, the first cellular division occurs before the introduced gene sequence has integrated into the cell's genome, the resulting animal will be a chimeric animal.

By breeding and inbreeding such animals, it has been possible to produce heterozygous and homozygous transgenic animals. Despite any unpredictability in the formation of such transgenic animals, the animals have generally been found to be stable, and to be capable of producing offspring which retain and express the introduced gene sequence.

The success rate for producing transgenic animals is greatest in mice. Approximately 25% of fertilized mouse eggs into which DNA has been injected, and which have been implanted in a female, will become transgenic mice.

AAV or retroviral infection can also be used to introduce a transgene into an animal. Here, AAV are preferred because high m.o.i. infections can result in multiple copies stably integrated per cell. Multiple copies of transgene are beneficial because: (a) increased level of transgene expression, (b) it reduces the chance that the target cell will lose or “kick out” the transgene, (c) transgene expression is not completely lost if one copy is mutated or inactivated and (d) it increases the likelihood of transgene expression in all lineages when the original target cell undergo any differentiation. The developing non-human embryo can be cultured in vitro to the blastocyst stage. During this time, the blastomeres can be targets for retroviral infection (Jaenich (1976) Proc. Natl. Acad. Sci USA 73:1260-1264). Efficient infection of the blastomeres is obtained by enzymatic treatment to remove the zona pellucida (Hogan, et al. (1986) in Manipulating The Mouse Embryo, Cold Spring Harbor Laboratory Press, Cold Spring Harbor, N.Y.). The viral vector system used to introduce the transgene is typically a replication-defective retrovirus carrying the transgene (Jahner et al. (1985) Proc. Natl. Acad. Sci. USA 82:6927-6931; Van der Putten et al. (1985) Proc. Natl. Acad. Sci. USA 82:6148-6152). Transfection is easily and efficiently obtained by culturing the blastomeres on a monolayer of virus-producing cells (Van der Putten, supra; Stewart et al. (1987) EMBO J. 6:383-388). Alternatively, infection can be performed at a later stage. Virus or virus-producing cells can be injected into the blastocoele (Jahner et al. (1982) Nature 298:623-628). Most of the founders will be mosaic for the transgene since incorporation occurs only in a subset of the cells which formed the transgenic non-human animal. Further, the founder may contain various retroviral insertions of the transgene at different positions in the genome which generally will segregate in the offspring. In addition, it is also possible to introduce transgenes into the germ line, albeit with low efficiency, by intrauterine retroviral infection of the mid-gestation embryo (Jahner et al. (1982) supra).

A third and preferred target cell for transgene introduction is the embryonic stem cell (ES). ES cells are obtained from pre-implantation embryos cultured in vitro and fused with embryos (Evans et al. (1981) Nature 292:154-156; Bradley et al. (1984) Nature 309:255-258; Gossler et al. (1986) Proc. Natl. Acad. Sci USA 83:9065-9069; and Robertson et al. (1986) Nature 322:445-448). Transgenes can be efficiently introduced into the ES cells a number of means well known to those of skill in the art. Such transformed ES cells can thereafter be combined with blastocysts from a non-human animal. The ES cells thereafter colonize the embryo and contribute to the germ line of the resulting chimeric animal (for a review see Jaenisch (1988) Science 240:1468-1474).

The DNA molecule containing the desired gene sequence may be introduced into the pluripotent cell by any method which will permit the introduced molecule to undergo recombination at its regions of homology. Transgenes can be efficiently introduced into the ES cells by DNA transfection or by retrovirus-mediated transduction.

In order to facilitate the recovery of those cells which have received the DNA molecule containing the desired gene sequence, it is preferable to introduce the DNA containing the desired gene sequence in combination with a second gene sequence which would contain a detectable marker gene sequence. For the purposes of the present invention, any gene sequence whose presence in a cell permits one to recognize and clonally isolate the cell may be employed as a detectable (selectable) marker gene sequence.

In one embodiment, the presence of the detectable (selectable) marker sequence in a recipient cell is recognized by hybridization, by detection of radiolabelled nucleotides, or by other assays of detection which do not require the expression of the detectable marker sequence. In one embodiment, such sequences are detected using polymerase chain reaction (PCR) or other DNA amplification techniques to specifically amplify the DNA marker sequence (Mullis et al. (1986) Cold Spring Harbor Symp. Quant. Biol. 51:263-273; Erlich et al. EP 50,424; EP 84,796, EP 258,017 and EP 237,362; Mullis EP 201,184; Mullis et al., U.S. Pat. No. 4,683,202; Erlich U.S. Pat. No. 4,582,788; and Saiki et al. U.S. Pat. No. 4,683,194).

Most preferably, however, the detectable marker gene sequence will be expressed in the recipient cell, and will result in a selectable or at least a detectable phenotype. Selectable markers are well known to those of skill in the art. Some examples include the hprt gene (Littlefield (1964) Science 145:709-710), the tk (thymidine kinase) gene of herpes simplex virus (Giphart-Gassler et al. (1989) Mutat, Res. 214:223-232), the nDtII gene (Thomas et al. (1987) Cell 51:503-512; Mansour et al. (1988) Nature 336:348-352), or other genes which confer resistance to amino acid or nucleoside analogues, or antibiotics, etc.

Thus, for example, embryonic cells which express an active HPRT enzyme are unable to grow in the presence of certain nucleoside analogues (such as 6-thioguanine, 8-azapurine, etc.), but are able to grow in media supplemented with HAT (hypoxanthine, aminopterin, and thymidine). Conversely, cells which fail to express an active HPRT enzyme are unable to grow in media containing HATG, but are resistant to analogues such as 6-thioguanine, etc. (Littlefield (1964) Science 145:709-710). Cells expressing active thymidine kinase are able to grow in media containing HAT, but are unable to grow in media containing nucleoside analogues such as bromo-deoxyuridine (Giphart-Gassler et al. (1989) Mutat. Res. 214:223-232). Cells containing an active HSV-tk gene are incapable of growing in the presence of gangcylovir or similar agents. This strategy can be useful following gene delivery to either ES cells or unfertilized eggs. The HSV-tk approach is especially suited to ES/blastocyst delivery or selection of developing zygotes since the “bystander effect” of tk (Freeman et al. (1996) Seminars in Oncology 23:31; Chen et al. (1995) Human Gene Therapy 6:1467) will kill not only the transduced cells but also the surrounding non-transduced cells. If genes are delivered to an unfertilized egg, both selection strategies can be applied, most suitably once fertilization has occurred and the cells begin to divide.

The detectable marker gene may also be any gene which can compensate for a recognizable cellular deficiency. Thus, for example, the gene for HPRT could be used as the detectable marker gene sequence when employing cells lacking HPRT activity. Thus, this agent is an example of agents may be used to select mutant cells, or to “negatively select” for cells which have regained normal function.

Chimeric or transgenic animal cells of the present invention are prepared by introducing one or more DNA molecules into a precursor pluripotent cell, most preferably an ES cell, or equivalent (Robertson in Capecchi, M. R. (ed.) (1989) Current communications in Molecular Biology, Cold Spring Harbor Press, Cold Spring Harbor, N.Y., pp. 39-44). The term “precursor” is intended to denote only that the pluripotent cell is a precursor to the desired (“transfected”) pluripotent cell which is prepared in accordance with the teachings of the present invention. The pluripotent (precursor or transfected) cell may be cultured in vivo, in a manner known in the art (Evans et al. (1981) Nature 292:154-156) to form a chimeric or transgenic animal. The transfected cell, and the cells of the embryo that it forms upon introduction into the uterus of a female are herein referred to respectively, as “embryonic stage” ancestors of the cells and animals of the present invention.

Any ES cell may be used in accordance with the present invention. It is, however, preferred to use primary isolates of ES cells. Such isolates may be obtained directly from embryos such as the CCE cell line disclosed by Robertson, E. J., In Capecchi, M. R. (ed.) (1989) Current Communications in Molecular Biology, Cold Spring Harbor Press, Cold Spring Harbor, N.Y., pp. 39-44), or from the clonal isolation of ES cells from the CCE cell line (Schwartzberg et al. (1989) Science 212:799-803). Such clonal isolation may be accomplished according to the method of Robertson in Robertson, E. J. (ed.) (1987) Teratocarcinomas and Embryonic Stem Cells: A Practical Approach, IRL Press, Oxford. The purpose of such clonal propagation is to obtain ES cells which have a greater efficiency for differentiating into an animal. Clonally selected ES cells are approximately 10-fold more effective in producing transgenic animals than the progenitor cell line CCE. An example of ES cell lines which have been clonally derived from embryos are the ES cell lines, AB1 (hprt⁺) or AB2.1 (hprt⁻).

In a preferred embodiment this invention utilizes Ola-derived E14 ES cells. The E14 embryonic stem cells are in the American Type Tissue Culture Repository at 12301 Parklawn Dr., Rockville, Md. USA, under accession number CRL 1821.

The ES cells are preferably cultured on stromal cells (such as STO cells (especially SNL76/7 STO cells) and/or primary embryonic G418 R fibroblast cells) as described by Robertson, supra. Methods for the production and analysis of chimeric mice are well known to those of skill in the art (see, for example, Bradley in Robertson, E. J. (ed.) (1987) Teratocarcinomas and Embryonic Stem Cells; A Practical Approach, IRL Press, Oxford, pp. 113-151). The stromal (and/or fibroblast) cells serve to eliminate the clonal overgrowth of abnormal ES cells. Preferably, the cells are cultured in the presence of leukocyte inhibitory factor (“lif”) (Gough et al. (1989) Reprod. Fertil. 1:281-288; Yamamori et al. (1989) Science 246:1412-1416).

ES cell lines may be derived or isolated from any species (for example, chicken, etc.), although cells derived or isolated from mammals such as rodents, rabbits, sheep, goats, fish, pigs, cattle, primates and humans are preferred. Cells derived from rodents (i.e. mouse, rat, hamster etc.) are particularly preferred.

### B) One or More Biological Activities of the Cell or Population of Cells is Monitored

The cells, tissues, organs, or organism transfected with the ribozyme library are then monitored for changes in one or more detectable characters. The particular character (activity) and the method of measuring it vary with the klind of gene under examination. For example, the methods of the invention are used to detect genes that mediate sensitivity and resistance to a selected defined chemical substance; examples include: drug toxicity genes; genes that encode resistance or sensitivity to carcinogenic chemicals; genes that encode resistance or sensitivity to infections with specific viral and bacterial pathogens. The methods of the invention are also used to detect unknown genes that mediate binding to a ligand, such as hormone receptors, viral receptors, and cell surface markers. The methods of the invention are also used to detect unknown tumor suppressor, transformation, and differentiation genes.

As indicated above, the particular target or character(s) under investigation determine the type of assay utilized. For example, the effects of ribozymes on nucleic acids that encode receptors (e.g., hormone or drug receptors, such as platelet-derived growth factor receptor (“PDGF”) is measured in terms of differences of binding properties, differentiation, or growth. Effects on transcription regulatory factors are measured in terms of the effect of ribozymes on transcription levels of affected genes. Effects on kinases are measured as changes in levels and patterns of phosphorylation. Effects on tumor suppressors and oncogenes are measured as alterations in transformation, tumorigenicity, morphology, invasiveness, adhesiveness and/or growth patterns. The list of type of gene function and phenotype that is subject to alteration goes on: viral susceptibility—HIV infection; autoimmunity—inactivation of lymphocytes; drug sensitivity—drug toxicity and efficacy; graft rejection—MHC antigen presentation, etc.

A number of biological characters monitored in target acquisition studies are illustrated in the examples. For example, tumorigenic cells are capable of growing on soft agar, while normal cells are not. Thus, cells (e.g. U138 cells) that have a tumor suppressor inhibited by a one or more ribozymes will develop a phenotype that allows growth on soft agar.

Effects of ribozymes on cellular differentiation can be assayed by changes in cell growth/proliferation, changes in surface proteins (sort by FACS), loss or gain of adherence/differential trypsinization, changes in cell size (sort by FACS), etc. Thus, for example PC12 cells whose differentiation is inhibited by ribozymes do not become post-mitotic and stop dividing.

Similarly genes that induce resistance to TRAIL can be identified by ribozymes that block apoptosis, and thus confer resistance to TRAIL and thereby allow the subject cells to proliferate.

Conversely, cell death is also a useful indicator. For example, cells that are drug resistant (e.g. multidrug resistant cancer cells) can be transfected by a ribozyme library and assayed for cell death in the presence of a cytotoxic drug (e.g. a cancer therapeutic such as cisplatin, vincristine, methotrexate, doxirubicin, etc.).

The foregoing list of characters is illustrative and not intended to be exhaustive. The variety of characters that can be screened in target acquisition studies is virtually limitless.

1) Use of controls in target acguisition assays.

It will be appreciated that where transfection with members of a ribozyme library, results in a alteration of a particular character/biological activity the change is typically measured with reference to an “unchanged” negative control and optionally a deliberately changes “positive” control. The use of such controls ]is well known to those of skill in the art. Typically negative controls are provided by an essentially identical cell, tissue, organ, or animal model that has not been transfected with the ribozyme library. A measurable difference, preferably a statistically significant difference between the control and the assay system indicates that a ribozyme has an effect.

It will be appreciated, however., that in selection systems, the fact of selection is its own control. Thus, for example where tumorigenic cells live and normal cells die (e.g. on soft agar) or drug resistant cells live while drug sensitive cells die, the simple fact of survival can indicate a significant alteration in a phenotypic character.

2) Distinguishing between ribozyme effects due only to binding to the target RNA as opposed to cleaving the RNA

Distinguishing between true catalytic activity and antisense activity is often desired in the selection of active ribozymes. Assays in cell culture allow selection of specific ribozymes out of the ribozyme library. Ribozymes initially selected inactivate expression of the target through either truly catalytic or simply antisense mechanisms. Less likely, although possible, the integration of the viral vector genome could disrupt gene function as well.

To confirm that an observed phenotype is ribozyme dependent (and not due to viral integration or to a spontaneous incidental mutation elsewhere in the genome), the viral-ribozyme genome is “rescued”. Thus, for example, an AAV-ribozyme genome is rescued from the host cell genome by transfection with a plasmid expressing the AAV viral proteins along with infection with wild type adenovirus. The AAV produced from these transfected/infected cells rescue and package the original AAV-ribozyme genome into new AAV particles. These are then used to infect fresh cells and assayed for loss of gene function. Ribozyme-dependent activity would continue to knock out the specific gene.

To verify that the ribozyme-dependent activity is due to catalytic rather than simply antisense, the selected ribozyme gene is structurally modified to abolish the cleavage activity without affecting substrate binding. This is also important so that a unique probe to the gene, including the GUC, can be generated. A three base mutation of AAA to CGU in loop 2 of the hairpin ribozyme (FIG. 1) has been identified that disables the ribozyme cleavage activity without disrupting its substrate binding (Anderson et al. (1994) Nucleic Acids Res. 22:1096; Ojwang et al. (1992) Proc. Natl. Acad. Sci. USA 89:10802). This mutation is then introduced into the selected ribozymes by PCR amplification using the 3′ disabled primer that contains the mutation. This new pool of “disabled” selected ribozymes is then re-introduced into AAV and assayed again for activity in cell culture. All AAV-disabled ribozyme clones that retain the ability to inactivate gene expression function through an antisense mechanism, while AAV-disabled ribozyme clones that lose this ability are indicative of an activity dependent on the ribozymes catalytic activity.

### C) Cells Showing a Change in the Monitored Activity (i.e., Due to Transfection with a Ribozyme Can be Isolated

Cells showing a change in the monitored activity due to transfection with a ribozyme can then be isolated according to standard methods known to those of skill in the art. Cells in in vitro culture can simply be physically isolated, and amplified, e.g. simply by spotting the appropriate transformed cells out into new culture medium.

Where the cells are present in a tissue, organ, or organism the cells can be isolated (e.g. by sacrifice of the organism if necessary) and homogenization of the tissue or organs to obtain free cells in suspension.

The cells can then be isolated e.g. visually where there is a visually detectable marker, by culture and selection, or by mechanical isolation e.g. by cell sorting (FACS).

### D) The Ribozyme or Ribozymes Contained in the Cells are Recovered

After application of the ribozyme library and selection of the desired phenotype, it is possible to “rescue” the responsible ribozyme(s) from the selected cells. The rescued ribozyme(s) are used both for re-application to fresh cells to verify ribozyme-dependent phenotype and for direct sequencing of the ribozyme to obtain the probe to be used for identifying the target gene.

In one approach, ribozyme genes may be rescued from tissue culture cells by either PCR of genomic DNA or by rescue of the viral genome (e.g., either AAV or RVV). To rescue by PCR cells are lysed in a lysis buffer containing a protease (e.g., proteinase K). The proteinase (e.g., proteinase K) is then inactivated (e.g., by incubation at 95° C. for 5 minutes). The ribozyme genes can then be isolated by PCR. Choice of PCR primers depends on the starting library vector and are designed to amplify from 200 bp to 500 bp containing the ribozyme sequence. The amplified Ribozyme fragment is then gel purified (agarose or PAGE).

This PCR product can be used for direct sequencing (fmole Sequencing Kit, Promega) or digested with BainHI and MluI and re-cloned into one of the Ribozyme expression plasmids. This PCR rescue operation can be used to isolate not only single ribozyme from a clonal cell population, but it can also be used to rescue a pool of ribozyme present in a phenotypically-selected cell population. After the ribozyme are re-cloned, the resulting plasmids can be used directly for target cell transfection or for production of viral vector.

A simpler and more efficient method for ribozyme rescue involves “rescue” of the viral genome from the selected cells by providing all necessary viral helper functions. In the case of retroviral vectors, selected cells are transiently transfected with plasmids expressing the retroviral gag, pol and amphotropic (or VSV-G) envelope proteins. Over the course of several days, the stably expressed LTR transcript containing the ribozyme is packaged into new retroviral particles, which are then released into the culture supernatant.

In the case of AAV, selected cells are transfected with a plasmid expressing the AAV rep and cap proteins and co-infected with wild type adenovirus. Here the stably-integrated AAV genome is excised and re-packaged into new AAV particles. At the time of harvest, cells are lysed by three freeze/thaw cycles and the wild type adenovirus in the crude lysate is heat inactivated at 55° C. for 2 hours. The resulting virus-containing media (from either the retroviral or AAV rescue) is then used to directly transduce fresh target cells to both verify phenotype transfer and to subject them to additional rounds of phenotypic selection if necessary to enrich further for the phenotypic ribozymes.

Similar to the PCR method described above, viral rescue of ribozyme allows for rescue of either single ribozyme or “pools” of ribozyme from non-clonal populations.

### E) The Collected Ribozymes are Optionally Expanded for Subsequent Rounds of Screening

As indicated above, the rescued ribozyme(s) are used both for re-application to fresh cells to verify ribozyme-dependent phenotype and for direct sequencing of the ribozyme to obtain the probe to be used for identifying the target gene. In addition, the rescue of “pools” of ribozyme from non-clonal populations provides a targeted ribozyme library that can be used for subsequent rounds of selection.

F) The Binding Sites of the Ribozymes Obtained from the First and/or Subsequent Rounds of Screening are Optionally Sequenced

The binding sites of the ribozymes obtained from the first and/or subsequent rounds of screening can be sequenced. The amplified constructs are relatively short (e.g. less than 500 nt) and can typically be fully sequenced in a single sequencing reaction. Methods sequencing nucleic acids are well known and kits containing reagents and instructions for such sequencing are commercially available from a wide variety of suppliers (see, e.g., fmole Sequencing Kit, Promega).

### G) Optionally the Sequence Information is Used to Search Sequence Databanks (e.g. GenBank) or to Design Probes to Specifically Identify and/or Isolate the Target(s) to which the Ribosome(s) Bound

1) Database searching.

The sequence information provided by sequencing the binding sites of the ribozyme(s) isolated as described above can be used to query nucleic acid databases. Such queries will identify sequences (present in the database) that contain binding sites recognized by the sequenced ribozymes. The information thus obtained may indicate the identity of the target or targets bound by the ribozyme(s) or it may be used to generate probes or target specific ribozyme libraries for further screening.

Methods of querying databases for sequence identity are well known to those of skill in the art. Standard algorithms (e.g., BLAST, GAP, BESTFIT, FASTA, TFASTA, PILEUP, etc.) are implemented by a wide variety of commercial software packages and internet web sites.

2) Isolation of nucleic acids

Using the sequence information provided from one or more ribozyme binding sites (RSTs) and possible additional information provided from database searches, there are various methods of isolating nucleic acid sequences that are or encode the target(s) to which the ribozymes bound (see Sambrook et al.). For example, DNA is isolated from a genomic or cDNA library by hybridization to immobilized oligonucleotide probes complementary to the desired sequences. Alternatively, probes designed for use in amplification techniques such as PCR are used, and the desired nucleic acids may be isolated using methods such as PCR. In addition, nucleic acids having a defined sequence may be chemically synthesized in vitro. Finally, mixtures of nucleic acids may be electrophoresed on agarose gels, and individual bands excised.

Methods for making and screening cDNA and genomic DNA libraries are well known. See Gubler, U. and Hoffman, B. J. (1983) Gene 25:263-269 and Sambrook et al, supra. To prepare a genomic library, the DNA is generally extracted from cells and either mechanically sheared or enzymatically digested to yield fragments of about 12-20 kb. The fragments are then separated by gradient centrifugation from undesired sizes and are constructed in bacteriophage lambda vectors. These vectors and phage are packaged in vitro, as described in Sambrook, et al. The vector is transfected into a recombinant host for propagation, screening and cloning. Recombinant phage are analyzed by plaque hybridization as described in Benton and Davis (1977) Science 196:180-182. Colony hybridization is carried out as generally described in M. Grunstein et al. (1975) Proc. Natl. Acad. Sci. USA., 72:3961-3965.

A cDNA library is generated by reverse transcription of total cellular mRNA, followed by in vitro packaging and transduction into a recombinant host.

DNA encoding a particular gene product is identified in either cDNA or genomic libraries by its ability to hybridize with nucleic acid probes, for example on Southern blots, and these DNA regions are isolated by standard methods familiar to those of skill in the art. See Sambrook et al.

Once a desired nucleic acid is detected in a mixture of nucleic acids, it is ligated into an appropriate vector and introduced into an appropriate cell, and cell clones that contain only a particular nucleic acid are produced. Preferably, strains of bacterial cells such as E. coli are used for cloning, because of the ease of maintaining and selecting bacterial cells.

PCR can be also used in a variety of protocols to isolate nucleic acids. In these protocols, appropriate primers and probes for amplifying a nucleic acid encoding a particular sequence are generated from analysis of the nucleic acid sequences listed herein. Once such regions are PCR-amplified, they can be sequenced and oligonucleotide probes can be prepared from the sequence obtained. These probes can then be used to isolate nucleic acid's encoding the sequence.

Other methods known to those of skill in the art may also be used to isolate particular nucleic acids. See Sambrook, et al. for a description of other techniques for the isolation of nucleic acid encoding specific protein molecules. Improved methods of cloning in vitro amplified nucleic acids are described in Wallace et al., U.S. Pat. No. 5,426,039. Other methods recently described in the art are the nucleic acid sequence based amplification (NASBA□, Cangene, Mississauga, Ontario) and Q Beta Replicase systems. These systems can be used to directly identify mutants where the PCR or LCR primers are designed to be extended or ligated only when a select sequence is present. Alternatively, the select sequences can be generally amplified using, for example, nonspecific PCR primers and the amplified target region later probed for a specific sequence indicative of a mutation.

### H) Detection of Nucleic Acid and Proteins

A number of embodiments of the present invention require detecting and quantifying specific nucleic acids, such as specific genes, RNA transcripts or ribozymes or protein products. For example, where the phenotypic character to be monitored is an mRNA, it may be desirable to detect and quantify a nucleic acid. Similarly where a phenotypic character to be monitored is a polypeptide, detection methods directed to polypeptides are appropriate.

1) Detection of nucleic acid presence and expression

A variety of methods for specific DNA and RNA detection and measurement, many involving nucleic acid hybridization techniques, are known to those of skill in the art. See Sambrook, et al.; Hames and Higgins (eds.) (1985) Nucleic Acid Hybridization, A Practical Approach, IRL Press; Gall and Pardue (1969) Proc. Natl. Acad. Sci. USA, 63:378-383; and John et al. (1969) Nature 223:582-587. The selection of a particular hybridization format is generally not critical.

Hybridization is carried out using nucleic acid probes which are designed to be complementary to the nucleic acid sequences to be detected. The probes can be full length or less than the full length of the target nucleic acid. Preferably nucleic acid probes are 20 bases or longer in length. Shorter probes are empirically tested for specificity. (See Sambrook, et al. for methods of selecting nucleic acid probe sequences for use in nucleic acid hybridization.)

For example, desired nucleic acids will hybridize to complementary nucleic acid probes under the hybridization and wash conditions of 50% :formamide at 42° C. Other stringent hybridization conditions may also be selected. Generally, stringent conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Typically, stringent conditions will be those in which the salt concentration is at least about 0.02 molar at pH 7 and the temperature is at least about 60° C. As other factors may significantly affect the stringency of hybridization, including, among others, base composition and size of the complementary strands, the presence of organic solvents and the extent of base mismatching, the combination of parameters is more important than the absolute measure of any one.

Oligonucleotides for use as probes are chemically synthesized, for example, according to the solid phase phosphoramidite triester method first described by Beaucage, S. L. and Carruthers, M. H., 1981, Tetrahedron Lett., 22(20):1859-1862 using an automated synthesizer, as described in Needham-VanDevanter, D. R., et al., 1984, Nucleic Acids Res., 12:6159-6168. Purification of oligonucleotides is by either native acrylamide gel electrophoresis or by anion-exchange HPLC as described in Pearson, J. D. and Regnier, F. E. (1983) J. Chrom. 255:137-149. The sequence of the synthetic oligonucleotide can be verified using the chemical degradation method of Maxam, A. M. and Gilbert, W. (1980) in Methods Enzymol. 65:499-560.

Typically, the probes used to detect hybridization are labeled to facilitate detection. Complementary nucleic acids or signal nucleic acids may be labeled by any one of several methods typically used to detect the presence of hybridized polynucleotides. The most common method of detection is the use of autoradiography with ³H, 125I, 35S, ¹⁴C, or ³²P-labeled probes or the like. Other labels include ligands which bind to labeled antibodies, fluorophores, chemiluminescent agents, enzymes, and-antibodies which can serve as specific binding pair members for a labeled ligand. (Tijssen, P., “Practice and Theory of Enzyme Immunoassays” in Burdon, R. H., van Knippenberg, P. H. (eds.) (1985) Laboratory Techniques in Biochemistry and Molecular Biology, Elsevier, pp. 9-20.)

One method for evaluating the presence or absence of particular nucleic acids in a sample involves a Southern transfer. Briefly, digested genomic DNA is run on agarose slab gels in buffer and transferred to membranes. Target nucleic acids are detected using labeled probes.

Similarly, a Northern transfer may be used for the detection of particular RNA molecules. In brief, total RNA is isolated from a given cell sample using an acid guanidinium-phenol-chloroform extraction method. The RNA is then electrophoresed to separate the RNA species and the RNA is transferred from the gel to a nitrocellulose membrane. As with the Southern blots, labeled probes are used to identify the presence or absence of particular RNAs.

An alternative means for determining the level of expression of a specific nucleic acid is in situ hybridization. In situ hybridization assays are well known and are generally described in Angerer, et al. (1987) Methods Enzymol. 152:649-660. In an in situ hybridization assay, cells are fixed to a solid support, typically a glass slide. If DNA is to be probed, the cells are denatured with heat or alkali. The cells are then contacted with a hybridization solution at a moderate temperature to permit annealing of labeled probes specific to the targeted nucleic acids. The probes are preferably labeled with radioisotopes or fluorescent reporters.

The sensitivity of the hybridization assays may be enhanced through use of a nucleic acid amplification system which multiplies the target nucleic acid being detected. in vitro amplification techniques suitable for amplifying sequences for use as molecular probes or for generating nucleic acid fragments for subsequent subcloning are known. Examples of techniques sufficient to direct persons of skill through such in vitro amplification methods, including the polymerase chain reaction (PCR) the ligase chain reaction (LCR), Q-replicase amplification and other RNA polymerase mediated techniques (e.g., NASBA) are found in Berger, Sambrook, and Ausubel, as well as Mullis et al. (1987) U.S. Pat. No. 4,683,202; Innis et al. (eds.) (1990) PCR Protocols A Guide to Methods and Applications, Academic Press, Inc., San Diego, Calif. (Innis); Arnheim & Levinson (Oct. 1, 1990) C&EN 36-47; (1991) J. NIH Res. 3:81-94; Kwoh et al. (1989) Proc. Natl. Acad. Sci. USA 86:1173; Guatelli et al. (1990) Proc. Natl. Acad. Sci. USA 87:1874; Lomell et al. (1989) J. Clin. Chem. 35:1826; Landegren et al. (1988) Science 241:1077-1080; Van Brunt (1990) Biotechnology 8:291-294; Wu and Wallace (1989) Gene 4:560; Barringer et al. (1990) Gene 89:117, and Sooknanan and Malek (1995) Biotechnology 13:563-564.

A preferred method of amplifying target sequences is the polymerase chain reaction (PCR). In PCR techniques, oligonucleotide primers complementary to the two 3′ borders of the nucleic acid region to be amplified are synthesized. The polymerase chain reaction is then carried out using the two primers. See Innis, M., Gelfand, D., Sninsky, J. and White, T. (eds.) (1990) PCR Protocols: A Guide to Methods and Applications Academic Press, San Diego. Primers can be selected to amplify the entire regions encoding a full-length ribozyme or selected subsequence, or to amplify smaller nucleic acid segments as desired.

2) Detection of protein gene products

Gene products such as polypeptides may be detected or quantified by a variety of methods. Preferred methods involve the use of specific antibodies.

Methods of producing polyclonal and monoclonal antibodies are known to those of skill in the art. See, e.g., Coligan (1991) Current Protocols in Immunology Wiley/Greene, NY; and Harlow and Lane (1989) Antibodies: A Laboratory Manual Cold Spring Harbor Press, NY; Stites et al. (eds.) Basic and Clinical Immunology (4th ed.) Lange Medical Publications, Los Altos, Calif., and references cited therein; Goding (1986) Monoclonal Antibodies: Principles and Practice (2d ed.) Academic Press, New York, N.Y.; and Kohler and Milstein (1975) Nature 256:495-497. Such techniques include antibody preparation by selection of antibodies from libraries of recombinant antibodies in phage or similar vectors. See, Huse et al. (1989) Science 246:1275-1281; and Ward et al. (1989) Nature 341:544-546. For example, in order to produce antisera for use in an immunoassay, an immunogen polypeptide or a fragment thereof is isolated or obtained as described herein. Mice or rabbits, typically from an inbred strain, are immunized with the immunogen protein using a standard adjuvant, such as Freund's adjuvant, and a standard immunization protocol. Alternatively, a synthetic peptide derived from proteins disclosed herein and conjugated to a carrier protein can be used as an immunogen.

Polyclonal sera are collected and titered against the immunogen protein in an immunoassay, for example, a solid phase immunoassay with the immunogen immobilized on a solid support. Polyclonal antisera with a titer of 10⁴ or greater are selected and tested for their cross reactivity against protein related or unrelated to the immunogen, using a competitive binding immunoassay. Specific monoclonal and polyclonal antibodies and antisera will usually bind to the immunogen with a K_{D} of at least about 0.1 mM, more usually at least about 1 micromolar, preferably at least about 0.1 micromolar or better, and most preferably 0.01 micromolar or better.

A number of immunogens may be used to produce antibodies specifically reactive with a particular peptide antigen. Recombinant protein is the preferred immunogen for the production of monoclonal or polyclonal antibodies. Naturally occurring protein may also be used either in pure or impure form. Synthetic peptides made using the sequences described herein may also used as an immunogen for the production of antibodies to the protein. Recombinant protein can be expressed in eukaryotic or prokaryotic cells as described above, and purified as generally described above. The product is then injected into an animal capable of producing antibodies. Either monoclonal or polyclonal antibodies may be generated, for subsequent use in immunoassays to measure the protein.

Methods of production of polyclonal antibodies are known to those of skill in the art. In brief, an immunogen, preferably a purified protein, is mixed with an adjuvant and animals are immunized. The animal's immune response to the immunogen preparation is monitored by taking test bleeds and determining the titer of reactivity to the immunogen. When appropriately high titers of antibody to the immunogen are obtained, blood is collected from the animal and antisera are prepared. Further fractionation of the antisera to enrich for antibodies reactive to the protein can be done if desired. (See, Harlow and Lane, supra).

Monoclonal antibodies may be obtained by various techniques familiar to those skilled in the art. Briefly, spleen cells from an animal immunized with a desired antigen are immortalized, commonly by fusion with a myeloma cell (See, Kohler and Milstein (1976) Eur. J. Immunol. 6:511-519 incorporated herein by reference). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate nucleic acid sequences which encode a monoclonal antibody or a binding fragment thereof by screening a DNA library from human B cells according to the general protocol outlined by Huse, et al. (1989) Science 246:1275-1281.

A particular protein can be measured by a variety of immunoassay methods. For a review of immunological and immunoassay procedures in general, see Basic and Clinical Immunology 7th Edition (D. Stites and A. Terr ed.) 1991. Moreover, the immunoassays of the present invention can be performed in any of several configurations, which are reviewed extensively in Maggio, E. T. (ed.) (1980) Enzyme Immunoassay, CRC Press, Boca Raton, Fla.; Tijssen, P. (1985) “Practice and Theory of Enzyme Immunoassays” in Laboratory Techniques in Biochemistry and Molecular Biology, Elsevier Science Publishers B. V. Amsterdam; and, Harlow and Lane, Antibodies, A Laboratory Manual, supra, each of which is incorporated herein by reference.

Immunoassays to peptides of the present invention may use a polyclonal antiserum which was raised to a defined protein, or a fragment thereof. This antiserum is selected to have low crossreactivity against other proteins and any such crossreactivity (for example, cross-reactivity against equivalent proteins from different species or tissues) is removed by immunoabsorbtion prior to use in the immunoassay.

In order to produce antisera for use in an immunoassay, the antigen protein, or a fragment thereof is isolated as described herein. For example, recombinant protein is produced in a transformed cell line. An inbred strain of mice such as balb/c is immunized with the selected protein of using a standard adjuvant, such as Freund's adjuvant, and a standard mouse immunization protocol. Alternatively, a synthetic peptide derived from the sequences disclosed herein and conjugated to a carrier protein can be used an immunogen. Polyclonal sera are collected and titered against the immunogen protein in an immunoassay, for example, a solid phase immunoassay with the immunogen immobilized on a solid support. Polyclonal antisera with a titer of 10⁴ or greater are selected and tested for their cross reactivity against proteins other than the antigen, using a competitive binding immunoassay such as the one described in Harlow and Lane, supra, at pages 570-573.

Immunoassays in the competitive binding format can be used for the crossreactivity determinations. For example, the selected protein can be immobilized to a solid support. Proteins (either distinct from, or related to, the antigenic protein) are added to the assay which compete with the binding of the antisera to the immobilized antigen. The ability of the above proteins to compete with the binding of the antisera to the immobilized protein is compared to the antigenic protein. The percent crossreactivity for the above proteins is calculated, using standard calculations. Those antisera with less than 10% crossreactivity with the antigenic proteins are selected and pooled. The cross-reacting antibodies are optionally removed from the pooled antisera by immunoabsorbtion with the above-listed proteins.

The immunoabsorbed and pooled antisera are then used in a competitive binding immunoassay as described above to compare a second protein to the immunogen protein. In order to make this comparison, the two proteins are each assayed at a wide range of concentrations and the amount of each protein required to inhibit 50% of the binding of the antisera to the immobilized protein is determined. If the amount of the second protein required is less than 10 times the amount of the immunogen protein that is required, then the second protein is said to specifically bind to an antibody generated to the immunogen protein.

The presence of a desired polypeptide (including peptide, transcript, or enzymatic digestion product) in a sample may be detected and quantified using Western blot analysis. The technique generally comprises separating sample products by gel electrophoresis on the basis of molecular weight, transferring the separated proteins to a suitable solid support, (such as a nitrocellulose filter, a nylon filter, or derivatized nylon filter), and incubating the sample with labeling antibodies that specifically bind to the analyte protein. The labeling antibodies specifically bind to protein on the solid support. These antibodies are directly labeled, or alternatively are subsequently detected using labeling agents such as antibodies (e.g., labeled sheep anti-mouse antibodies where the antibody to a protein is a murine antibody) that specifically bind to the labeling antibody.

IV. Generation of Target Specific Libraries

Another application of the present invention is the generation of target specific libraries. Most RNA targets (viral RNA, cellular mRNA, etc.) are relatively large (i.e. >1 kb) and the sequence is not always known, especially if the target RNA is generated from genomic DNA fragments deduced by population genetics and restriction fragment length polymorphisms (RFLPs). In addition, we have found secondary structure within certain RNA targets to be a serious hindrance to ribozyme cleavage (Welch et al. (1996) Gene Therapy 3:994). Historically, functional ribozyme cleavage sites have been deduced by brute force, synthesizing individual ribozymes one at a time and assaying their activity on a large target RNA in vitro. Furthermore, in many instances, ribozymes that cleave in vitro do not cleave in vivo (Welch et al. (1996) Gene Therapy 3:994).

One goal of this technology is to start with a substantially complete high complexity “library” of ribozymes, containing all possible target recognition sequences and select and enrich for specific ribozymes most active at inactivating the expression of a specific gene or ablating a specific gene function in vivo.

The selection procedures are performed as described above. In this instance, a population of ribozymes is recovered (rescued) (e.g. from non-clonal cells) and pooled and expanded (amplified) to form a target specific library enriched for specific ribozymes most active at specifically binding and cleaving the target(s).

V. In vitro Identification of Efficient Site-specific Ribozymes from a Random Ribozyme Library and the Generation of Target Specific Libraries

Some applications contemplate the in vitro identification of efficient site-specific ribozymes prior to their in vivo expression. Additionally, when the target RNA is large, it may be desirable to create a library of ribozymes each with specificity for different sites within the same target (a “target-specific” library). While these can be accomplished by a number of known methods, two preferred methods are further described. The first takes advantage of the inherent ability of the hairpin ribozyme to catalyze a trans-ligation reaction between the products of the cleavage reaction. By creating a self-cleavable ribozyme library, the trans-ligation reaction will join the specific ribozyme to one of its cleavage products. The ligated ribozymes now can be selectively amplified out of the library. The second preferred method is to immobilize the target RNA on a solid support, thus allowing soluble ribozymes to be selected based on their ability to bind, cleave and elute off of the target. The target can be any RNA (e.g. cellular or viral RNA), or DNA that has been converted to RNA. It is preferable to immobilize the target RNA by its 5′ end (see below), but RNA immobilized via its 3′ end is also suitable. Since both of these methods will positively select and amplify only actively cleaving ribozymes, they are far superior to previously published and patented methods such as brute force cloning of individual ribozymes (Welch et al. (1996) Gene Therapy 3:994) or the construction of “quasi-random” ribozymes (Draper et al. (1996) U.S. Pat. No. 5,496,698). These considerations become especially important if the target RNA is large (e.g. hepatitis C virus RNA ˜9.5 kb) and/or has an unknown sequence (e.g. large chromosomal DNA fragments converted to RNA).

### A) Trans-ligation of Specific Ribozymes to Their Cleavage Products

The hairpin ribozyme is capable of cleaving a target RNA in both a cis and trans configuration (Bruening et al. (1988) Structure and Expression, 1:.239-248; Hampel et al (1988) Biochem. 28:4929). It also has the ability to readily catalyze the reverse of the cleavage reaction and religate the cleavage products to reform the original substrate RNA (Hegg et al. (1995) Biochemistry 34: 15813; Joseph et al. (1993) Genes and Development 7:130). In fact, in the presence of an excess of cleavage products the ligation reaction is favored over that of the cleavage reaction by a factor of ten (Hegg et al. (1995) Biochemistry, 34:15813).

This ligation reaction can be applied in the generation of target specific libraries. An elegant and efficient method for accomplishing this task is to make use of the ribozyme as a molecular tag. This ribozyme tag will provide a universal upstream primer for the subsequent isolation and amplification of the reaction products. This will facilitate the identification and sequence determination of the unique cleavage sites present within the target RNA and be used to generate a target specific ribozyme gene vector library.

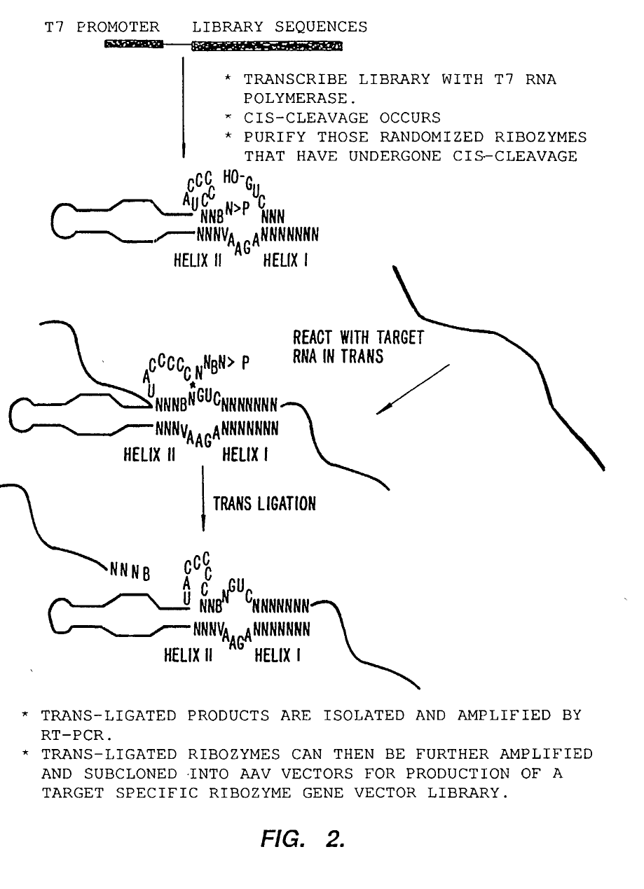

To utilize the ribozyme as a molecular tag, the ribozyme must be capable of catalyzing trans-ligation at the site of cleavage within the target RNA. This can be accomplished by designing a combinatorial ribozyme library that first undergoes an autolytic cleavage. This self-processed library is then incubated in a trans-cleavage reaction with the target RNA of interest and, with a certain frequency, the ribozymes will become covalently attached to the target RNA at the site if cleavage through trans-ligation (FIG. 2). Specifically, a ribozyme combinatorial library will be constructed wherein the inter-molecular helices I and II will be completely randomized. This library will also contain, attached to its 3′ end, a completely randomized cis-cleavage site having only a 3bp helix I and helix II. The cis-cleavage site is tethered to the 3′ end of the ribozyme by means of a 5 bp polypyrimidine tract. The ribozyme library is transcribed and concurrently will undergo the cis-cleavage reaction. This will generate a pool of randomized ribozymes also having a randomized helix II cleavage product still attached to the ribozyme. The presence if this helix II cleavage product is important for two reasons. The first being, as a localized source of readily available helix II cleavage products suitable for ligation and the second, is the fact that the helix II cleavage product contains the 2,3, cyclic phosphate necessary for providing the energy required to drive the ligation reaction. This pool of “helix II charged ribozymes” is then purified from the rest of the library and used in a trans-cleavage reaction with the target RNA under standard cleavage conditions. The ribozyme will cleave the target at specific sites and, with a certain frequency, the ribozyme will become covalently attached to the target RNA at these sites by means of the trans-ligation reaction (FIG. 2).

The identification of these unique cleavage sites is then determined by RT-PCR. The reaction products from the trans-cleavage reaction are reacted with polyA-polymerase to generate a polyA tail on the 3′ end of the reaction products. The RNA is then reverse transcribed using oligo-dT as the primer. This resulting cDNA is then amplified by PCR using the oligo dT as the downstream primer and a universal upstream primer provided by the ligated ribozyme sequence. The reaction products are amplified by PCR and can be sequenced directly or after subcloning. To generate a target specific ribozyme gene vector library, the selected ribozyme genes are further cloned into AAV vectors.

### B) Immobilizing Target RNA Via its 5′ End

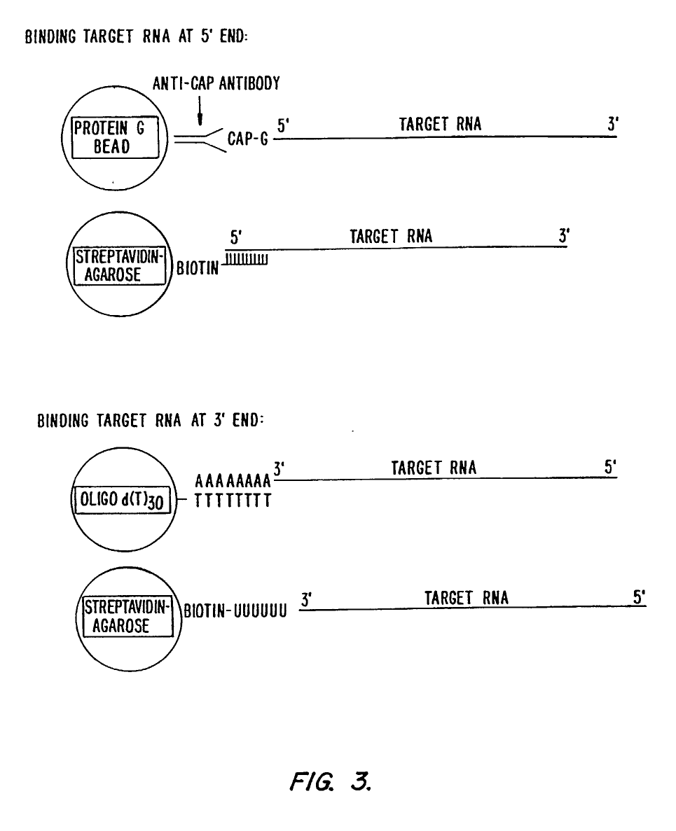

If the target RNA has a 5′ methyl-G cap (such as cellular mRNA and many viral RNAs), the RNA can be immunoprecipitated using monoclonal antibodies directed against the cap structure (Garcin and Kolakofsky (1990); Weber, 1996) and immobilized on Protein G sepharose beads (Pharmacia, Uppsala, Sweden) (see FIG. 3). If the target RNA is not capped (such as some viral RNAs, non-messenger cellular RNA or RNA transcribed in vitro), it can be bound to streptavidin-agarose beads (Pierce, Rockford Ill.) via a 30-mer oligonucleotide that is biotinylated at its 3□ end (see FIG. 3). The sequence of the 30-mer is complementary to the 5∇ end of the target RNA. If the target is a known viral or cellular RNA, the oligo is designed based on the known sequence of the RNA's 5′ end. If the target RNA comes from genomic DNA of unknown sequence that has been converted to RNA via retrovirus packaging, the oligo is designed based on the retroviral-specific immediate 5′ sequence transcribed from the LTR. Likewise DNA cloned into in vitro transcription vectors and transcribed by T7 RNA polymerase to yield the target, are engineered to contain specific 30 nt at their 5′ end, upstream of the actual target sequence. In general, then, the 3′ end of the specific 30-mer biotinylated oligo is bound to the streptavidin column and the 5′ 30 nt bind the target RNA by Watson-Crick base pairing (see FIG. 3). To prepare the column, the biotinylated oligo is incubated with the beads and unbound oligo is washed out. The target RNA is then mixed with the oligo column, heated to 95° C. and cooled slowly to allow annealing of the oligo and target RNA. The column is then washed to remove unbound target RNA.

### C) Immobilizing Target RNA Via its 3′ End

It is occasionally necessary to immobilize the target RNA by its 3′ end. If the target RNA is polyadenylated mRNA, a simple oligo d(T)₃₀ column would bind the target RNA (Pharmacia) (FIG. 3). If the target RNA is not polyadenylated (or if one wishes a stronger binding than simple Watson-Crick basepairing), the 3□ end of the RNA can be biotinylated using biotin-UTP (Sigma, St. Louis, Mo.) and terminal transferase (Promega, Madison, Wis.), according to the manufacturers. The biotinylated target can then be immobilized on streptavidin-agarose beads (Pierce, Rockford Ill.) (FIG. 3).

### D) Ribozyme Library Preparation

This application involves the use of a library of randomized ribozymes as opposed to randomized ribozyme genes. In vitro synthesis of the ribozymes encoded by the library is accomplished by transcribing the double-stranded ribozyme gene library (described in Specific Example a.) with T7 RNA polymerase, as described (Welch, P. J., et al. (1996) Gene Therapy 3:994-1001). For later tracking and selection purposes, the ribozyme library can be transcribed in the presence of trace amounts of P-32 UTP. The ribozyme library transcription reaction is then treated with DNase to remove the DNA template. Lastly, transcribed ribozymes are purified by polyacrylamide gel to enrich for full length transcripts. If desired, the ribozymes can be radio-labeled with [³²P]UTP, which can be used as a marker to follow the binding of the ribozymes at various stages of selection (see below).

### E) Ribozyme Library Selection

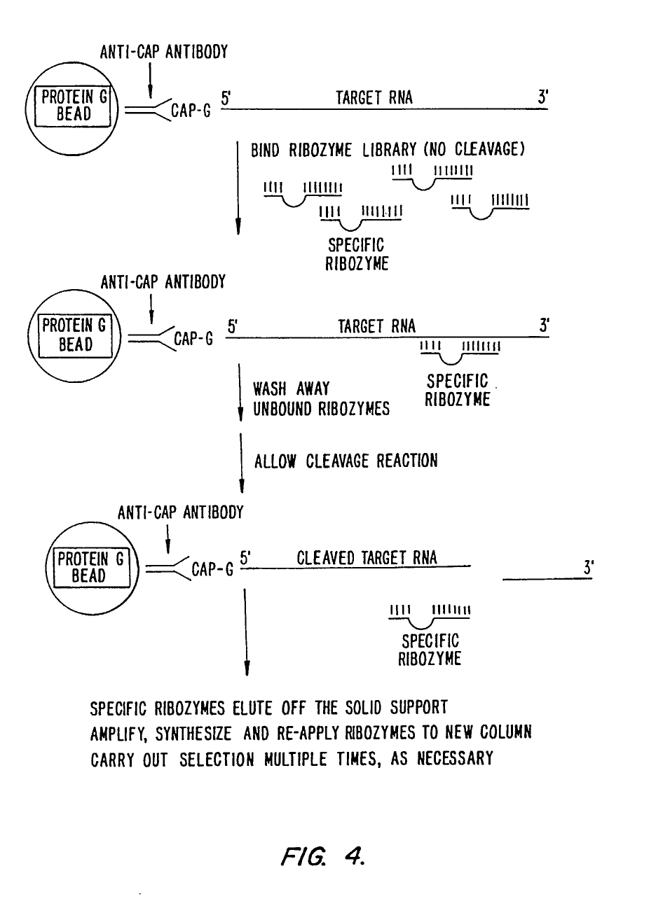

The RNA target column is pre-treated with non-specific RNA (such as E. coli rRNA or yeast tRNA), the ribozymes are loaded in the absence of magnesium, and the unbound non-specific RNA washed from the column (FIG. 4). This reduces non-specific binding of ribozyme to the column. The ribozyme library is then added to the RNA target column along with non-specific RNA, again in the absence of magnesium, thus allowing ribozyme binding without actual cleavage of the target RNA (Ojwang et al. (1992) Proc. Natl. Acad. Sci. USA 89:10802).

For tracking and selection purposes, the ribozyme library can be transcribed in the presence of trace amounts of ³²P-UTP, thus allowing quantitation of ribozyme binding and release throughout all the selection steps. The ribozyme library is added such that the target RNA is in molar excess, otherwise more than one ribozyme will be released from the column following a successful cleavage, generating false-positive results. The column is then washed free of unbound ribozyme.

Specific ribozyme binding can be monitored by following the radioactivity remaining bound to the column. Magnesium-containing ribozyme cleavage buffer is then added to the column and the slurry is incubated at 37° C. for two hours to allow for substrate cleavage to occur. When a ribozyme successfully cleaves the target, it temporarily acts as a “bridge” between the 5′ and 3′ substrate products. Since the 5′ product is bound by only a 4 bp helix, this interaction rapidly melts at 37° C. and the ribozyme is released from the solid support (FIG. 4). If the target RNA is immobilized via its 3′ end, the cleaving ribozyme remains bound by the 7 bp helix, which will also rapidly melt at 37° C. (max Tₘ˜22° C.). Therefore, all “released” ribozymes are ones with activity against the target. These are then eluted and precipitated for amplification. Again, the specificity of the binding and cleavage reactions can be monitored by following the radioactivity present in the transcribed ribozymes. For proof that the selection procedure is successful, the initial library can be “spiked” with a known amount of purified ribozyme with known activity against the target (if available).

### F) PCR Amplification of Selected Ribozymes

Reverse transcriptase is used to convert the selected ribozyme pool to DNA using a primer specific to the 3′ end of all the ribozymes (3′ Primer). This primer includes the MluI site and a portion of the common region of the ribozyme and is therefore present in all ribozymes which were made in the library. The reverse transcriptase products are then amplified by standard PCR using a primer specific for the 5′ end of all ribozymes in the library including a BamHI restriction site (5′ Primer). This 5′ primer used in this amplification step may or may not also include (at its 5′ end) a T7 promoter arm for a future transcription steps. The PCR products are then purified and transcribed with T7 RNA polymerase. The resulting “selected” ribozymes are gel purified, and then used for a second (third, fourth, etc.) round of further selection on a fresh target column, bound, allowed to cleave and subsequently eluted and amplified as above until only specific, active ribozymes remain in the pool. Ribozyme binding and activity is continually monitored by following the location of the radiolabeled pool of ribozymes, and this is also used as a measure of specificity of the selection. For example, with an unselected pool of ribozyme the majority of the radiolabel will not even bind the column. Conversely, with a highly selected pool, most of the radiolabel would initially bind the column and then most would be released once magnesium was added. To avoid loss due to radioautolysis, ribozyme transcription, binding and selection is performed in one day. The subsequent PCR amplification products do not contain any radioactive nucleotides, and are therefore stable for long periods of time. Together, the combination of high-specificity binding and subsequent PCR amplification allows for conditions that are both selective and of high yield.

### G) Ribozyme Cloning, Sequencing, Identification of Sites and Target Gene Cloning

Once satisfied with the selected pool of ribozymes, each specific ribozyme is cloned from its amplified double-stranded DNA template into a sequencing vector (e.g., pGem7Z, Promega) via the BamHI and MluI sites. Each ribozyme clone is then sequenced and the resulting sequence of the ribozyme binding arms is used to identify the site within the target (if the target sequence is known) or to generate a DNA probe to clone the target gene (if the gene is unknown). To construct such a probe, the sequence of the ribozyme binding arms is combined with the requisite GUC to construct a DNA probe 5′-XXXXXXGACNXXXX-3′ (where X is the deduced sequence coming from the specific ribozyme), which is then used to screen cDNA libraries to clone the gene.

### H) Selection Enhancement

If multiple rounds of selection on the same column still yield false positives due to release of inactive ribozymes bound downstream of an active one, the selected ribozymes are then applied to another column prepared with the RNA target bound to the column in the reverse orientation (i.e. if target bound on 5□ previously, then switch to 3 □ immobilization). This re-screening and amplification is repeated as many times as necessary to satisfy pre-determined requirements set for the ribozymes to be selected (i.e. diversity of ribozyme number, ribozyme efficiency, total ribozyme number, etc.) If P-32 UTP is included in the ribozyme transcripts, as mentioned previously, the binding ratio of those ribozymes which remain bound to the target RNA on the column relative to that which has cleaved the target RNA can be tracked from screening to screening. Again, as selection progresses, this ratio will steadily shift greater for ribozymes which cleave the target RNA instead of remaining bound to the target. Furthermore, screening success can be quantified by the number of PCR cycles required to amplify the selected ribozymes (Conrad et al. (1995) Molecular Diversity 1:69). As the ribozyme pool is further selected and amplified, the number of required PCR cycles would be expected to reduce proportionally.

### I) Assembling Target-specific Ribozyme Gene Vector Libraries

Once the target-specific ribozymes have been selected, amplified and identified, the ribozyme genes are cloned into AAV vectors, resulting in a specific ribozyme gene vector library (see previous and later sections for cloning and application). The ribozyme fragment generated after PCR amplification contains BamHI and MluI restriction sites (see 5′ Primer and 3′ Primer). Digestion with the two enzymes not only generates cohesive ends for easy cloning into AAV vectors but also removes the T7 polymerase promoter sequences. Once generated, this library of AAV-ribozyme can be used for a variety of applications including, but not limited to, therapeutic and gene functional analysis in vivo.

VI. Differential Ribozyme Gene Libraries

Frequently, when analyzing different cell types, it is necessary to determine how gene expression differs between the two cell types. For example, when attempting to determine the cause of tumor formation, one often wishes to compare gene expression between a transformed cell and its parental cell type. Other examples include cells before and after viral infection, or following a cell through various stages of differentiation. Previous methods for isolating such differentially-expressed genes (briefly described below) are time consuming, technically challenging and often yield many false positive results. Immusol's ribozyme library technology not only removes these disadvantages, but also results in a functional ribozyme or ribozymes that can immediately be used to knockout the gene or genes in question, for functional analysis.

Historically, a procedure called “subtractive hybridization” would be employed to determine which genes are differentially expressed (for review Ausubel, F., et al. (ed.) (1987) Current Protocols in Molecular Biology, Greene Publishing and Wiley-Interscience, New York. Briefly, mRNA or cDNA from each cell type are mixed and allowed to hybridize. The hybridized products (dsRNA or dsDNA) are then removed by column chromatography and the remaining, unhybridized nucleic acids (the differentially-expressed genes) are cloned. The main disadvantages, among others, of this method lies in its technical difficulty and its time consuming procedures.

More recently, a method called “differential display” has been developed (for review see Ausubel, F., et al. (ed.) (1987) Current Protocols in Molecular Biology, Greene Publishing and Wiley-Interscience, New York. Briefly, partially random primers are used in PCR to amplify a subset of mRNAs expressed in each cell type. The PCR products are then separated by polyacrylamide gel electrophoresis and the amplified bands between the two cell types are compared. Unique bands are excised from the gel, re-amplified and cloned. The main disadvantages of this method are that each PCR reaction only targets a subset of differentially-expressed genes. Indeed, many different primer sets (and subsequent PCR reactions) are required for a full representation of all mRNA species. In addition to generating many false-positives, differential display is really only suitable for detecting medium- to high-abundance mRNAs.

In one embodiment, the high complexity substantially complete randomized ribozyme libraries of the present invention are used in vitro to both identify differentially-expressed genes and to generate specific, active ribozymes against the unique mRNAs. To accomplish this, mRNA is isolated from the two different cell lines in question (cell A and cell B). Individual target RNA columns are prepared for each cell type by either: a) binding the mRNAs by their 5′ ends using a monoclonal antibody directed against the 5′ methyl-G cap (for detailed discussion see above section on identification of ribozymes that cleave a known target RNA), bound to protein G-sepharose or b) binding the mRNAs by their 3′ polyadenylated tails to an oligo(dT) column.

The ribozyme library is synthesized by in vitro transcription and applied to the column prepared from the mRNA of cell A under conditions that inhibit cleavage such as the absence of magnesium or low temperature (thus allowing ribozyme binding but not cleavage). Ribozymes that flow through this column represent targets not present in the mRNA pool of cell A. The bound ribozymes are then allowed to cleave by changing the conditions to favor cleavage (i.e. add magnesium or increase temperature). Active, specific ribozymes are then released from the solid support. Ribozymes that are released at this step are ones capable of both binding and cleaving RNA from cell A. These ribozymes are then applied to the RNA column from cell B under conditions that prevent cleavage. These cell A-specific ribozymes that also bind the cell B column represent ribozymes that recognize RNA targets present in both cells, while the ribozymes that flowthru are ones that recognize RNA only expressed in cell A. These ribozymes are then amplified, cloned and sequenced to produce a probe to clone the differentially-expressed, cell A-specific genes. Additionally, the specific ribozymes are cloned into vectors (e.g. AAV vectors) which can be applied to cell A to analyze the effects and function of the differentially-expressed genes. Naturally, the above described process can be reversed (i.e. apply ribozymes to column B first then column A) to isolate genes differentially expressed in cell B.

Additionally, more than one differential selection method can be employed. For example, differential display could be used to generate RNA fragments specific for one cell type, and these RNA's could then be used to generate a target specific library.

### A) In vivo Selection of Optimal Ribozyme(s) Against a Defined Target

Target cells are generated that express the target RNA of interest. If the product of the target gene itself is FACS-sortable (i.e. any cell surface protein that is detectable by a specific antibody) or is selectable by various culturing methods (i.e. drug resistance, viral susceptibility, etc.), then one can proceed directly to application of the vector library below. If not, then the target gene sequence is cloned in cis to two separate reporter genes that are either FACS-sortable and/or selectable, for example the green fluorescent protein (GFP) or the nerve growth factor receptor (NGFR) that are FACS-sortable and HSV thymidine kinase (tk) that renders a cell sensitive to gancyclovir. These two target-reporter constructs are then stably transfected into cells (e.g. HeLa or A549) to create the target cells.

The AAV vectors in which the ribozyme library is embedded contain a neo^{r} gene as a selection marker and for titering purposes. Target cells are grown to 70-80% confluency and transduced with the AAV-ribozyme library at an m.o.i.>1 (to favor multiple transduction events, and multiple ribozyme genes, per cell). Transduction is accomplished by incubating cells with vector overnight at 37° C., as described above. Transduced cells are selected by culturing the cells for 10-14 days in the presence of G418 (400-500 micrograms/ml culture medium).

To determine which cells are expressing ribozymes directed against the target, the transduced cells are sorted and/or selected for the two cis-linked reporter genes (or for the specific gene product if it itself is sortable/selectable). In the reporter system, two different reporters are necessary to distinguish between ribozymes specific for the target or simply recognizing the reporter itself. Cells in which the expression of both reporter genes is reduced are then believed to express ribozymes specific for the target.

The ribozyme vectors present in these surviving cell clones are rescued from the cell by wild type AAV or by transient transfection with packaging plasmids in the presence of adenovirus (Harmonat and Muzyczka (1984) Proc. Natl. Acad. Sci. USA 81:6466; Tratschin et al. (1985) Mol. Cell. Biol. 5:3251; Samulski et al.(1982) Proc. Natl. Acad. Sci. USA 79:2077). The rescued vectors are then re-introduced into the untransduced parental cell line under conditions favoring a single ribozyme pro-vector per cell, and reselected or screened.

Once a cell line containing a single specific ribozyme gene is thus deconvoluted, identified, and cloned, the corresponding ribozyme gene found within the cell line is PCR cloned and sequenced using PCR primers described herein. The resulting sequence is expected to be exactly complementary to the gene sequence the ribozyme is inactivating, except that the target RNA must also contain

VI. Kits

In still another embodiment, this invention provides kits for the practice of the methods of this invention. The kits preferably comprise one or more containers containing a substantially complete high complexity ribozyme gene library and/or ribozyme vector library of this invention. The kit can optionally additionally include buffers, culture media, vectors, sequencing reagents, labels, antibiotics for selecting markers, and the like.

The kits may additionally include instructional materials containing directions (i.e., protocols) for the practice of the assay methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are offered to illustrate, but not to limit the claimed invention.

### Example 1

### Construction of Full-length AAV Random Ribozyme Library Provector

We generated random ribozyme-gene libraries in pAMFT.dBam and pAGU5 vectors using multiple rounds of polymerase chain reaction (PCR) with primers of ribozyme sequences containing randomized nucleotides in the substrate binding sites. The protocol is illustrated in FIG. 5.

Initially, the randomized ribozyme oligonucleotides were made according to the standard industrial procedure which involved delivering ¼ amount each of A, T, C, G phorphoramidites in the synthesis column to synthesize each N (where N represents a “doped” position ¼A, ¼C, ¼G, and ¼T). In this approach, the automated synthesizer had to deliver equal amounts each of the A, T, C, and G dispersers 11 times to make an oligonucleotide population containing 11 Ns. When the randomized oligonucleotides synthesized in this manner were sequenced, it was discovered that A, T, C, and G were frequently not equally incorporated into the N positions as shown in Table 1.

TABLE 1
Distribution of four nucleotides in degenerated region
of library with oligonucleotide prepared by
conventional “doping”.

| Nt. | Helix I | | | | | | | AGAA | Helix II | | | | Total |
|---|---|---|---|---|---|---|---|---|---|---|---|---|---|
| G | 7 | 6 | 9 | 6 | 5 | 5 | 4 | | 9 | 9 | 7 | 8 | 75 |
| A | 1 | 2 | 1 | 4 | 0 | 2 | 2 | | 0 | 0 | 1 | 1 | 14 |
| T | 2 | 1 | 0 | 0 | 4 | 2 | 3 | | 0 | 0 | 0 | 1 | 13 |
| C | 0 | 1 | 0 | 0 | 1 | 1 | 1 | | 1 | 1 | 2 | 0 | 8 |

Therefore, to ensure the random (uniform) incorporation of A, T, C, and G nucleotides in the helix 1 and helix 2 region where the N nucleotide is represented, the A, T, C ,G, reagent was premixed and the same mixture was used for every N position in the oligonucleotide synthesis. Since the premixing utilizes a substantially larger amount of A, T, C, and G nucleotide reagents is done only once for the oligonucleotide synthesis, the randomized distribution of the each A, T, C, and G was much more reliable than that made in the standard procedure.

Sequences of library oligonucleotides made in this way confirmed that distribution of A, T, C, and G in the randomized region of ribozymes are more uniform as illustrated in Table 2.

TABLE 2
Distribution of four nucleotides in degenerated region
of library with oligonucleotides prepared according
to the modified procedure

| Nt. | Helix I | | | | | | | | AGAA | Helix II | | | | Tot. |
|---|---|---|---|---|---|---|---|---|---|---|---|---|---|---|
| G | 11 | 9 | 9 | 11 | 8 | 6 | 5 | 7 | | 11 | 9 | 7 | 5 | 98 |
| A | 1 | 4 | 3 | 4 | 7 | 6 | 6 | 5 | | 7 | 5 | 5 | 4 | 57 |
| T | 12 | 7 | 10 | 6 | 6 | 7 | 8 | 6 | | — | 6 | 10 | 6 | 84 |
| C | 2 | 6 | 4 | 5 | 5 | 7 | 7 | 8 | | 8 | 6 | 3 | 11 | 72 |

### A) First Round PCR

A fragment comprising an AAV 3′ ITR, a tRNAval promoter, and ribozyme library genes was produced by PCR using the primers set P1 and P2 where P1 is a 3′ AAV-ITR primer (41 nt) (5′-AGG AAG ATC TTC CAT TCG CCA TTC AGG CTG CGC AAC TGT TG-3′ (SEQ ID NO: _) and P2 is a 5′-oligonucleotide with sequences for a tRNAval promoter and ribozyme library genes (72 nt) (5′-ATA CCA CAA CGT GTG TTT CTC TGG TNN NNT TCT NNN NNN NGG ATC CTG TTT CCG CCC GGT TTC GAA CCG GGG-3′).

A fragment comprising an AAV 5′ ITR, a ribozyme library gene, and a neo selection marker was produced by PCR using the primers set P3, an oligonucleotide containing ribozyme library gene complementary to the P2 oligonucleotide (72 nt) 5′-CCC CGG TTC GAA ACC GGG CGG AAA CAG GAT CCN NNN NNN AGA ANN NNA CCA GAG AAA CAC ACG TTG TGG TAT (SEQ ID NO: _) and P4 a 5′ AAV-ITR primer (40 nt) (5′-AGG AGA TCT GCG GAA GAG CGC CCA ATA CGC AAA CCG CCT C-3′ (SEQ ID NO: _).

### B) The Second Round of PCR

The resulting PCR products from the first round of PCR were purified and used as templates for a second round PCR using P1 and P4 primers to generate the full length AAV vector with ribozyme library gene.

### C) Analysis of the Complexity of the Ribozyme Library

The complexity and function of the ribozyme library was analyzed by in vitro cleavage of known target substrates, which included two PCN1 targets (PCN3 and PCN4), one HIV target pol3308, novel anti-HIV Ribozymes, HBV and one HCV target. As shown in FIG. 6, the ribozyme library contains a high degree of sequence complexity as determined by its ability to cleave 5 different RNA substrates known to be cleavable by corresponding ribozymes.

### Example 2

### Construction of AAV Plasmid Ribozyme Library

### A) AAV Ribozyme Library (pAAV6Clib) with 7 Random Nucleotides in the Helix 1 Region Driven by the tRNAval Promoter

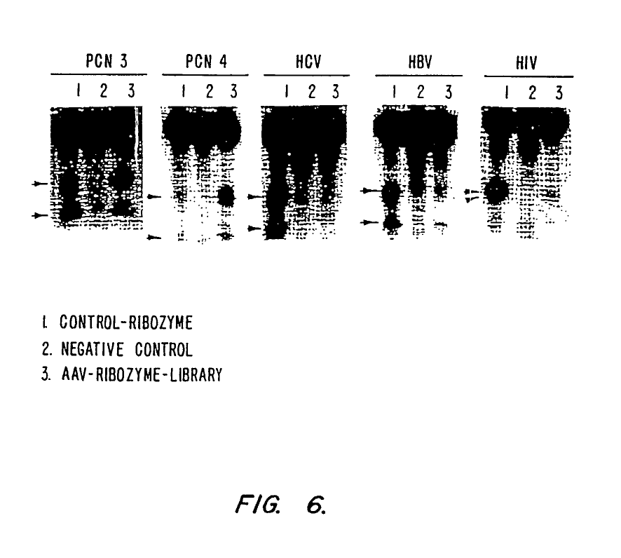

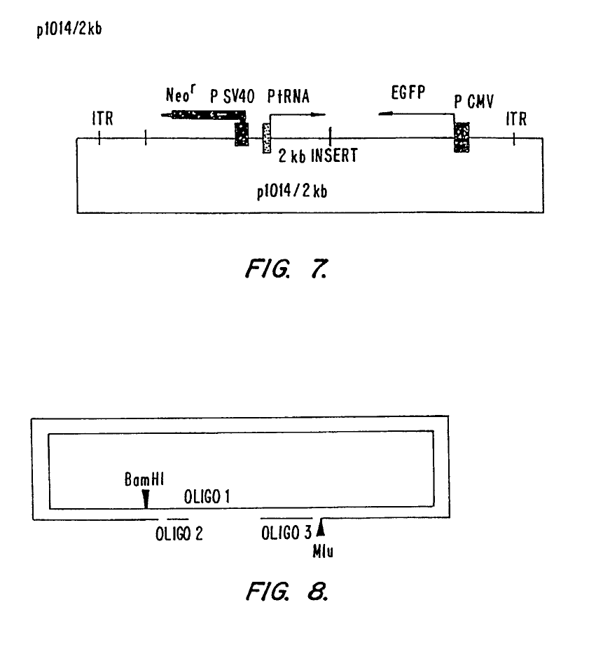

The vector p1014-2k (FIG. 7) was used for cloning a library of ribozyme genes. Plasmid p1014-2k is a recombinant plasmid carrying: 1) 5′ and 3′ inverted terminal repeats (ITR) of adeno-associated viral genome; 2) neomycin resistance marker driven by a SV40 promoter; 3) eGFP Florescence marker downstream of a CMV promoter; 4) transcription cassette for the ribozyme genes via tRNAval promoter with a 2 kb insert between Bam HI and Mlu I sites.

The following parameters are crucial to achieve full complexity of ribozyme library in AAV vector. 1) Randomized oligonucleotides containing the ribozyme sequence; 2) Increased transformation efficiency of host (e.g., bacteria); 3) The elements for efficient packaging of AAV library DNA into virion are and remain intact during the library construction.

To ensure the starting oligonucleotides contain truly randomized ribozyme substrate binding sites, the “doped” oligonucleotides were made as described in Example 1. To increase the transformation efficiency of the host bacteria used in library construction, increased the library transformation efficiency as well and substantially reduced the background transformation due to the vector itself.

To increase the overall transformation efficiency we optimized ration of 3 oligos with the linearized vector, the ligation conditions, the procedures for electroporation, and the choice of the most efficient competent cells DH12S.

To reduce the background transformation due to the vector itself, we put a 2Kb insert in between the BamH I and Mlu I cloning sites in the AAV vector. It was a discovery of this invention that background “noise” (transformants lacking the ribozyme insert) observed during library construction is due to the presence of the uncut vector as well as single enzyme digested vector. Inserting additional nucleic acid (e.g. the 2 kb insert in between the two restriction enzymes sites) allowed us to easily isolate the 8 kb fragment which was completely digested by the two enzymes from the 10 kb fragment derived from single digestion or uncut vector.

The large (2 Kb) insert was also is designed to eliminate vector from being packaged (due to its 6 kb size in between two ITRs) because DNA more than 5.8 kb can not be packaged in virions in rAAV production. Many reports show that ITR regions of the AAV vector is crucial for producing high titer of AAV as well as achieving stable transduction (Muzyczka (1992) Curr. Topics Microbiol. Immunol. 158, 97-129). Therefore, to ensure ITRs are intact in the AAV library, we checked for any possible deletions which may cause both inefficient package and stable transduction using the restriction enzyme BssH II before and after AAV library construction. The intact ITR will give a single DNA fragment of 85 base pairs while any deleted ITR will have one or more fragment less than 85 base pairs. In addition to that, we grew the bacteria culture for AAV library production at 30° C. to decrease the deletion rate of ITRs.

More specifically, p1014-2k (100 μg) was thoroughly digested overnight at 37° C. with restriction enzymes BamHI and MluI (200 units each). The digested DNA was fractionated by agarose gel electrophoresis. An 8 kb fragment was extracted from the gel. 0.2 pmol of the 8 kb fragment was ligated with 3 oligonucleotides: (oligo 1: Oligo 1: 5′-pGAT CCA CCC CCC NNN NNN NAG AAN NNN ACC AGA GAA ACA CAC GTT GTG GTA TAT TAC CTG GTA-3′ (SEQ ID NO: _), Oligo2: 5′-pGGG GGG TG-3′ (SEQ ID NO: _, and Oligo 3: 5′-pCGG GTA CCA GGT AAT ATA C-3′ (SEQ ID NO: _) as illustrated in FIG. 8 at a molar ration of 1:3:30:30 (8 kb fragment: oligo1:oligo2:oligo3). Ligation was performed using 10 units of ligase at 16° C. overnight. All of the oligonucleotides were phosphorylated at the 5′ end to ensure high ligation efficiency.

Efficiency of transformation by ligated DNA via electroporation in DH12S cells (GIBCO) was determined by counting numbers of transformed bacterial colonies formed per transformation with ligated DNA. A total of 2.9×10⁷ number of transformants were obtained for the library. Thirty randomly picked individual clones from the library were sequenced to evaluate the quality of the library. There were no repeats of sequences in substrate binding regions of ribozymes.

### B) AAV Ribozyme Library (pAAVPGKlib) with 8 Random Nucleotides in the Helix 1 Region and a Tetraloop in the Loop 3 Region Under the Control of PGK Promoter

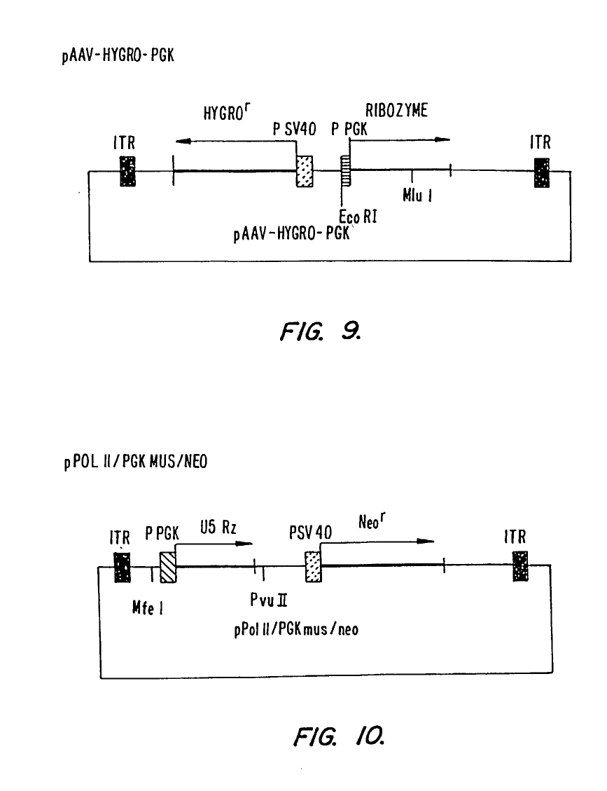

An AAV vector plasmid pAAVhygro-PGK (FIG. 9) was used to clone a library of ribozyme genes driven by a PGK promoter. The PGK promoter was chosen because of its high promoter activity in driving the ribozyme against HIV U5 region which resulted in the best anti HIV effect in cell culture as shown in Table 19 We also incorporated tetraloop feature in ribozyme to increase ribozyme activity in vivo based on the data obtained from anti-fusin ribozymes. Ten ribozymes were tested for activity against CXCR-4 in HeLa cells. Each ribozyme was constructed in a native and tetraloop configuration. Ribozyme genes were stably introduced in HeLa cells by rAAV transduction and G418 selection using the rAAV construct pAMFTdBam. we found that none of the ten native ribozymes were effective in reducing the level of CXCR-4 expression on the cells as assayed by FACS. On the other hand two of the tetralooped ribozymes, CR4184 and CR415, significantly reduced CXCR-4 expression.

The substrate binding site in the helix 1 region was randomized for 8 nucleotides to cover potentially more potent ribozymes without losing achievable complexity.

The plasmid was constructed as follows. First, a hygromycin resistance gene was copied from plasmid pCDNA3.1 (Invitrogen) by PCR and cloned into an AAV vector plasmid backbone (pSUB201) to generate plasmid pAAV/hygro. Two restriction sites Spe 1 and EcoR V were placed up stream of an SV40 promoter, which controls the transcription of the hygromycin resistant gene, to facilitate subsequent cloning of the ribozyme library into the plasmid.

To assure that the hygromycin resistant gene copied by PCR has the right sequence, plasmid pAAV/hygro was transfected into HeLa cells followed by hygromycin selection. Once the resistance to hygromycin was confirmed, a DNA fragment containing the U5 ribozyme transcription unit under the control of PGK promoter was cut from plasmid pPolII/PGKmus/neoBHGPA (FIG. 10) and cloned into pAAV/hygro such that the transcription of the hygromycin resistance gene and that of ribozyme are towards opposite directions. Afterward, a 3 kb DNA fragment was used to replace the BamHI and MluI fragment of U5 ribozyme-coding region. The resulting plasmid pAAVhygro-PGK was digested completely with BamHI and MluI and gel purified. Three oligonucleotides: Oligo 4: 5′-pAAT TCT GCA GAT ATC CAT CAC ACT GGC GGG GAT CCT CGA GNN NNN NNN AGA ANN NNA CCA GAG AAA CAC ACG GAC TTC GGT CCG TGG TAT ATT ACC TGG TA-3′ (SEQ ID NO: _), Oligo 5: 5′-pCTC GAG GAT CCC CGC CAG TGT GAT GGA TAT CTG CAG-3′ (SEQ ID NO: _), and Oligo 6: 5′-pGCG TAC CAG GTA ATA TAC CAC GGA CCG AAG TCC GTG TGT TTC TCT GGT-3′ (SEQ ID NO: _) were then ligated to the linearized vector according to the protocol described above to generate pAAVhygro-pGK-lib. The complexity of the ribozyme library containing 8 randomized nucleotides in helix 1 and 4 nucleotides in helix 2 is 4⁴⁺⁸, 2×10⁷. The number of individual bacterial colonies in the library is 8×10⁷, which is the about 98% of chance of having 2×10⁷.

### C) AAV Ribozyme Library (pAAVlib) with 8 Random Nucleotides in the Helix 1 Region and a Tetraloop in the Loop 3 Region Under the Conrol of tRNAval Promoter

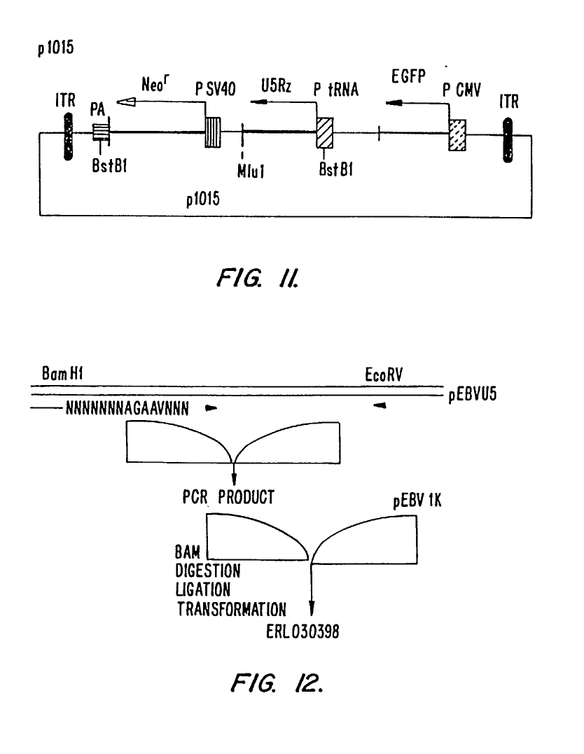

The vector plasmid p1016 for ribozyme library cloning is a derivative of plasmid p1015 (FIG. 11), which contains the DNA sequences encoding selection markers EGFP and aminoglycoside phosphotransferase. Plasmid p1015 has two Bst B1 sites. One is in the tRNAᵥₐₗ promoter region and the other is located at 20 bases down stream of the stop codon of neo^{r} mRNA.

In order to use Bst B1 and Mlu sites to clone the ribozyme library into the plasmid by the three oligonucleotide cloning method described above, one Bst B1, which is located down stream of the neo^{r} mRNA stop codon, was removed to generate p1015sBst. Then a 2 kb DNA fragment was inserted into the modified plasmid 1015sBst to replace the BamHI and MluI fragment of U5 ribozyme-coding region to make p1016.

The expression of neo^{r} in Hela cells was tested for plasmid p1016 to assure that the neo^{r} was not mutated. After digestion with BamHI and MluI, the 8 Kb fragment containing p1016 backbone was ligated with 3 oligonucleotides: Oligo 7: 5′-pCGA AAC CGG GCG GAA ACA GGA TCC NNN NNN NNA GAA NNN NAC CAG AGA GAA ACA CAC GGA CTT CGG TCC GTG GTA TAT TAC CTG GTA-3′ (SEQ ID NO: _), Oligo 8: 5′-pGGA TCC TGT TTC CGC CCG GTT T-3′ (SEQ ID NO: _), and oligo 3: 5′-pCGC GTA CCA GGT AAT ATA CCA CGG ACC GAA GTC CGT GTG TTT CTC TGG T-3′ (SEQ ID NO: _) to generate pAAVlib by the method described above.

After ligation, 1/10 volume of 5 M ammonium acetate and 1/40 volume of 2 mg/ml glycogen were added to the ligation solution. After brief vortex, 2.5 volume of ethanol was added. The solution was then kept at −70° C. for one hour followed by centrifugation at 14,000 rpm for 20 min in a microcentrifuge. The DNA pellet was washed three times with 70% ethanol and then dried for 3 min in a spin-vacuum dryer. The pellet was resuspended in a small aliquot of water to a concentration of 1 μg/μL. For electroporation, 1 μL of plasmid DNA (1 μg) was mixed with 80 μL of DH12S electroporation competent cells (from GIBCO). The cells were then transferred into a electroporation cuvette.

Electroporation was carried out at 1.7 kV, 25 μF and 200 ohm. Afterwards, 1 ml of SOC was added to the cells. After agitated at 37° C. for one hour, the cells were plated on two 15 cm agarose plates with 100 μg/ml ampicillin. Under the optimized conditions described above, we can get 3.3×10⁷ or more colonies by one electroporation. The total complexity of the finished library was 3.6×10⁸, which is 5 times more colonies to cover 99% of the complexity of the library. The background of the library was less than 5% as judged by digestion with restriction enzymes. The randomness of the library was confirmed by direct DNA sequencing. The results showed that there are no repeat ribozymes in 18 randomly picked individual clones. The distribution of the four bases A, T, C, and G appeared equal (Table 3).

TABLE 3
The distribution of ATCG in the helix 1 and
helix 2 region (master library)

| | POSITIONS | | | | | | | | | | | | |
|---|---|---|---|---|---|---|---|---|---|---|---|---|---|
| Base | 1 | 2 | 3 | 4 | 5 | 6 | 7 | 8 | 13 | 14 | 15 | 16 | % |
| A | 5 | 4 | 4 | 2 | 4 | 3 | 4 | 3 | 3 | 2 | 3 | 1 | 20 |
| T | 7 | 4 | 3 | 4 | 6 | 6 | 6 | 5 | 6 | 8 | 2 | 2 | 31 |
| C | 1 | 2 | 6 | 6 | 2 | 5 | 1 | 4 | 7 | 3 | 3 | 5 | 23 |
| G | 3 | 6 | 3 | 4 | 4 | 2 | 5 | 4 | 0 | 3 | 8 | 8 | 26 |

### Example 3

### Construction of EBV Plasmid Ribozyme Library

### A) EBV Plasmid Ribozyme Libraries ERL030398 with 8 Random Nucleotides in the Helix 1 Region Driven by tRNAval Promoter

Certain viral DNA sequences can direct plasmid DNA into eukaryotic cells to be maintained as an episome form. The Epstein-Barr virus (EBV) episome is one of the well characterized systems. There are four advantages for using EBV libraries to identify unknown genes associated with phenotype changes: 1) In most primate and human cells, the presence of EBV EBNA-1 protein will support the replication of plasmid DNA carrying an EBV origin of DNA replication. Since the episomes are maintained as multi-copy DNA (usually 100-200 copies/cell), this system results in higher level of gene expression than single copy gene construct, which is beneficial for knocking down a target mRNA. It thus improves the potential success of selecting of desired phenotypic changes. 2) The episomes can be easily rescued and multiple round of selection of phenotypic change can be easily achieved. 3) The use of a simple plasmid based vector will preserve the complexity of the ribozyme library by eliminating the virus production step associated with AAV or retroviral vectors. 4) They are also valuable for cells that are resistant to viral vector transduction.

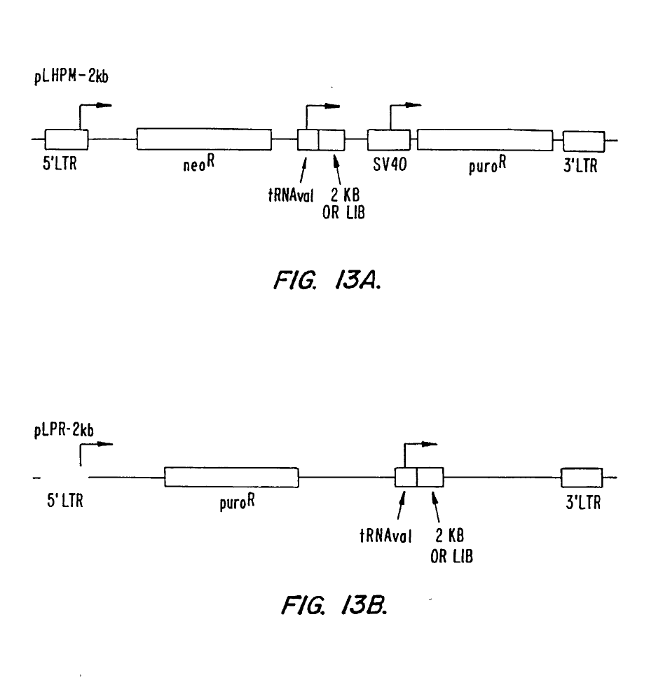

To construct the EBV plasmid ribozyme library, we obtained plasmid vector pREP4 from Invitrogen, that contains the EBV EBNA-1 gene and the EBV origin of replication as well as a hygromycin resistant gene expression cassette driven by the HSV TK promoter. A ribozyme cassette, U5 ribozyme against HIV1 (Mang et al. (1994) Proc. Natl. Acad. Sci. USA, 90: 6340-6344) driven by tRNA promoter, was placed in the polylinker region of pREP4. The resulting plasmid was named pEBVU5. Plasmid pEBVU5 contains an unique Bam HI site right in front of the helix I of ribozyme and unique Eco RV site about 735 basepairs down stream of the ribozyme sequence. The ribozyme library was generated by PCR reaction using the pEBVU5 as template with two primers, libbam and EBVlibeco (FIG. 12). The primer libbam contains degenerated oligonucleotide in the helix I and helix II of ribozyme sequence. The sequences of these two primers are libbam (5′-CCC CCG GGG GAT CCN NNN NNN NAG AAV NNN ACC AGA GAA ACA CAC GGA CTT CGG TCC GTG GTA TAT TAC CTG GTA CGC GTT TTT GCA TTT TT-3′ (SEQ ID NO: _)) and EBVlibeco (5′-TGG GGT GGG AGA TAT CGC TGT TCC TTA (SEQ ID NO: _)).

The PCR reactions were carried out with 1×10⁶ copies of pEBVU5, 0.1 μM of each primer, and 1 U of Taq DNA polymerase in 100 μl reaction mixture. The PCR condition were: 94° C. 4′ for pre-PCR, and 35 cycles of 94° C. 30″, 47.5° C. 30″, and 72° C. 1′. The PCR products were purified using Qiagen's PCR purification kit and used for EBV ribozyme library construction.

To eliminate U5 in the library, a new vector backbone plasmid, pEBV1k, was constructed by inserting about 1 kb DNA Bam HI fragment from pAV2 (ATCC No. 37216) into the Bam HI site of pEBVU5. During the construction of ERL030398, about 200 μg of plasmid pEBV1k and 20 μg of PCR product from above were digested with 500 units each of restriction enzymes of Bam HI and Eco RV in a total volume of 2 mls in Promega buffer D at 37° C. for 4 hrs. After digestion, 250 units of alkaline phosphatase were added to digested pEBV1k tube and the reactions were allowed to proceed for another 30 min. at 37° C. The enzymes were heat inactivated for 30 min at 37° C. and, the reaction mix was cleared by centrifugation at 14,000 rpm for 20′.

The clear supernatants were transferred to fresh tubes for ligation. The ligation reaction of 1 ml contains 200 μl of T4 DNA ligase buffer and 50 unit of T4 ligase from GIBCO/BRL, 10 μg of Bam HI and Eco RV digested pEBV1k and 1 μg of Bam HI and Eco RV digested PCR product. The ligation reaction lasted 4 hrs at room temperature. At the end of ligation, the DNA was precipitated with 2 volume of ethanol in the presence of 10% original volume of ammonium acetate on dry ice/ethanol bath for 1 hr. The DNA was recovered by centrifugation and washed with 70% ethanol and dried briefly in speed vacuum. The resulting DNA pellet was resuspended in 200 μL of distilled, sterile water.

Two microliters of ligation mixture were mixed with 40 μL of electro-competent DH10B cells on ice and the mixture were transferred in to 0.1 cm cuvette for electroporation. The condition of electroporation was 1700V, 200 Ohms, and 25 μF. The electroporated bacteria were incubated with 1 ml of LB medium at 37° C. for 1 hr. and plated into LB agar plates containing ampicillin. A total of 120 transformations were carried out and estimated efficiency of transformation was about 1.1×10⁶ colonies/transformation/μg of DNA. The total independent colonies for EBVRZLIB030398 was about 1.32×10⁸, which has a 99.5% chance to include the full complexity the library. All the colonies were pooled and frozen at −80° C. in aliquot of 1 ml with 1.0×10¹⁰ bacteria.

TABLE 4
Distribution of four nucleotides in degenerated region
of ERL030398

| Nt. | Helix I | | | | | | | | AGAA | Helix II | | | | Tot. |
|---|---|---|---|---|---|---|---|---|---|---|---|---|---|---|
| G | 11 | 9 | 9 | 11 | 8 | 6 | 5 | 7 | | 11 | 9 | 7 | 5 | 98 |
| A | 1 | 4 | 3 | 4 | 7 | 6 | 6 | 5 | | 7 | 5 | 5 | 4 | 57 |
| T | 12 | 7 | 10 | 6 | 6 | 7 | 8 | 6 | | | 6 | 10 | 6 | 84 |
| C | 2 | 6 | 4 | 5 | 5 | 7 | 7 | 8 | | 8 | 6 | 3 | 11 | 72 |

### Example 4

### Construction of Retroviral Plasmid Ribozyme Library

Two plasmid-based retroviral ribozyme libraries were created to contain 8 random nucleotides in helix 1 and 4 random nucleotides in helix 2. Both vectors have ribozyme expression driven by the tRNAval promoter. pLHPM-Lib contains antibiotic resistance to neomycin and puromycin and the pLPR has the tetraloop addition in the ribozyme and expresses puromycin resistance.

It is important to create libraries with a variety of selection markers (or none at all) since different cell systems will have different requirements. For example, some reporter cell lines may already be neomycin resistant due to the stable introduction of the reporter, thus puromycin selection would be necessary for stable selection of the library. Or, if the target cell has a re-introduced chromosome or some other unstable element that requires continued neomycin selection to maintain, having a library with only puromycin would allow double selection for both reporter and Rz library.

The parental vector pLHPM-2 kb (FIG. 13a) contains: 1) 5′ and 3′ long terminal repeats (LTR) of the Moloney retroviral genome; 2) neomycin resistance driven by the LTR; 3) transcription cassette for the ribozyme genes via tRNAval promoter with 2 kb insert at ribozyme location; and 4) SV40 promoter driving puromycin resistance.

The parental vector pLPR-2 kb (FIG. 13b) contains: 1) 5′ and 3′ long terminal repeats (LTR) of the Moloney retroviral genome; 2) puromycin resistance driven by the LTR; and 3) transcription cassette for the ribozyme genes via tRNAval promoter with 2 kb insert at ribozyme location.

To generate the ribozyme library, either parental vector (pLHPM-2 kb or pLPR-2 kb) was thoroughly digested overnight at 65° C. with restriction enzyme BstBI (400 units). The DNA was then extracted with phenol:chloroform and ethanol precipitated. Resuspended DNA was digested overnight at 37° C. with 400 units of MluI and the 6 kb vector DNA was purified by agarose gel electrophoresis. This excises the 2 kb stuffer fragment and allows easy separation of vector from the 2 kb, as well as from any undigested or linearized parent plasmid. We found this to be critical for reducing background colonies after ligation of the library.

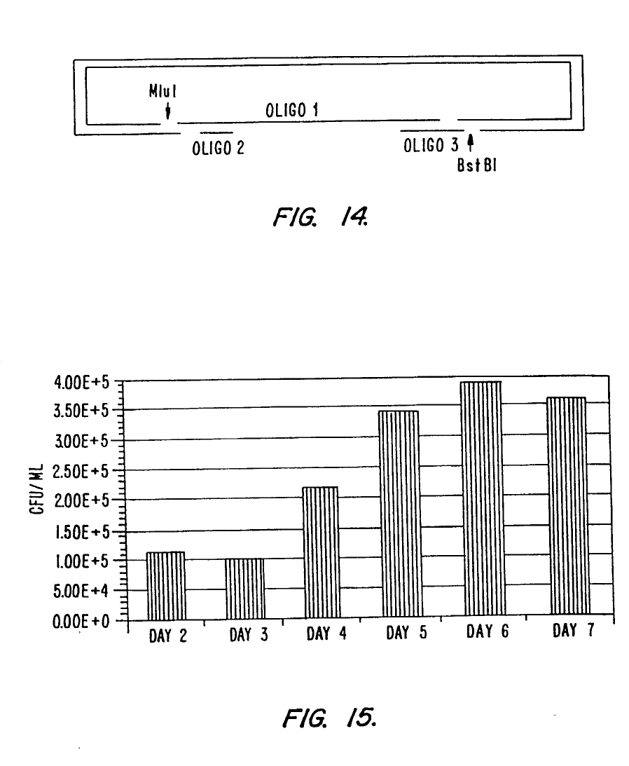

To create the ribozyme library insert, three oligonucleotides were annealed in annealing buffer (50 mM NaCl, 10 mM Tris pH 7.5, 5 mM MgCl₂) at a molar ratio of 1:3:3 (oligo1:oligo2:oligo3) by heating to 90° C. for 5 minutes followed by slow cooling to room temperature as shown in FIG. 14. The oligonucleotides were Oligol 5′-pCGC GTA CCA GGT AAT ATA CCA CGG ACC GAA GTC CGT GTG TTT CTC TGG TNN NNT TCT NNN NNN NNG GAT CCT GTT TCC GCC CGG TTT-3′ (SEQ ID NO: _), Oligo2,5′-pGTC CGT GGT ATA TTA CCT GGT A-3′ (SEQ ID NO: _), and Oligo3,5′ pCGA AAC CGG GCG GAA ACA GG-3′ (SEQ ID NO: _).

The randomness introduced into Oligo 1 was obtained by chemical synthesis using a “hand-mix” of nucleotides as described in Example 1 to assure equal distribution of all four possible nucleotides at each random position. In addition, the oligonucleotides are synthesized with a 5′ phosphate, which is critical for efficient ligation. We have found that chemical addition of the 5′ phosphate is much more efficient and more easily controlled than enzymatic addition using T4 polynucleotide kinase.

For the ligation, 0.5 pmole of the 6 kb vector and an 8-fold molar excess of annealed library oligonucleotides were ligated overnight with 10 units of T4 DNA ligase (see below). Having an 8-fold molar excess of insert to vector also proved very important since we discovered that less insert:vector caused vector to reclose without any insert (as measured by the destruction of both restriction sites), thus increasing the background of empty vector. This phenomenon was due to our extremely high ligation and transformation efficiencies.

Ultracompetent bacteria were produced (see specific Example for their production and transformation) and transformed with the ligation mixture. Efficiency of ligation was determined by counting numbers of transformed bacterial colonies formed per transformation with ligated DNA. A total of 5×10⁷ bacterial colonies were obtained for the library. 25 individual clones from the library were sequenced to evaluate the “randomness” of the library (see specific Example for statistical assessment of randomness). The bacterial colonies were pooled in aliquots as a master stock and frozen at −80° C. The working stocks were made by culturing 1 ml of the master stock in 60 ml LB media overnight at 30° C. 1 ml of the working stock was used to make 500 ml bacterial culture by incubation at 30° C. overnight. DNA is then extracted from the 500 ml culture for the subsequent retroviral library production.

Incorporation of all of the above discoveries allowed us to create a plasmid-based retroviral ribozyme library. To illustrate, the following Table 5 contains our previous attempts at generating such complexity, leading to the final protocol resulting in 5×10⁷ bacterial colonies per transformation.

TABLE 5
Progression in development of high complexity libraries.

| TRANSFORMATION | COLONIES PER TRANSFORMATION |
|---|---|
| 1 | 1.2 × 10⁴ |
| 2 | 6 × 10⁴ |
| 3 | 2.3 × 10⁴ |
| 4 | 3.4 × 10³ |
| 5 | 6 × 10⁵ |
| 6 | 5 × 10⁷ |

### Example 5

### Creation and Transformation of Ultracompetent Bacterial Cells

Generation of a sufficiently complex ribozyme plasmid library requires bacteria of extremely high competency. Bacterial electroporation typically yields the highest transformation efficiency so electrocompetent cells were generated from the strain DH12S by the following protocol. DH12S were streaked cells onto an LB plate and the next day single colony was inoculated into 5 ml of LB broth. The 5 ml culture was allowed to grow overnight at 37° C. and in the morning 2.5 ml of the culture was diluted into 500 ml of LB broth. The bacteria was grown at 37° C. until it reached an OD₆₀₀ of between 0.5 and 0.6.

The cells were then chilled in an ice water bath for 15 minutes before harvesting at 4200 rpm in a Beckman J-6M rotor at 2° C. The cells were resuspended in 5 ml of ice cold sterile water, then 500 ml of ice cold water was added the resulting solution well mixed. The cells were incubated in an ice water bath for 10 minutes. This incubation in the cold increased the competency of the cells. The centrifugation and incubation in ice cold water was repeated.

During this time, microcentrifuge tubes were pre-chilled in a dry ice/ethanol bath. The cells were harvested again and then resuspended in 50 ml of ice cold 10% glycerol. The cells were centrifuged again the volume of the pellet was estimated.

The cells were resuspended in an equal volume of ice cold 10% glycerol. 300 μL of cells was aliquoted into each of the prechilled microcentrifuge tubes which were then stored at −80° C.

These electrocompetent cells must be extremely competent in order to generate a library of sufficient complexity. The cells are electroporated with a Bio-Rad Gene Pulser® II with a capacitance of 25 μF and a resistance of 200 ohms. The competency level of the cells is always tested by transforming them with a supercoiled plasmid and at least 1×10¹⁰ transformants per μg of DNA must be obtained for the cells to be used for library transformations, because the ligated ribozyme library will not transform as efficiently as supercoiled DNA. To be sure we had the most highly competent cells possible, we compared our cells head to head with ElectroMAX DH12S™ cells from Gibco/BRL. Our cells consistently gave more transformants when identical transformation conditions were carried out.

### Example 6

### Retroviral Vector Ribozyme Library Production, Purification and Characterization

### A) Retroviral Vector Ribozyme Library Production

When using retroviral vector to deliver the ribozyme library it is important to produce an abundant amount with a high enough titer level to maintains the complexity of the ribozyme library. A transient transfection method was developed and optimized because a stably expressing producer cell line could not cover the complexity of the ribozyme library for two reasons: 1) Greater than 2×10⁷ different ribozymes must be transfected, stably integrated and then maintained as a fully complex library; and 2) Any ribozyme in the library that happened to be toxic or detrimental to the packaging cell line would be automatically pre-selected out of the library, thus reducing the complexity prior to every generating the viral library.

A clone of canine thymus cells (Cf2A12) was identified based on its ability to produce high titer retrovirus. These cells were seeded at 3.2×10⁴ cell/cm² one day prior to transfection in a cell factory (total volume=1000 ml). Approximately 24 hours later the cells were transfected with Transit-LT1 (Mirus Corp.) and three plasmids. Plasmid number one contained the ribozyme library and two selectable markers, neomycin and puromycin. Plasmid number two contained retrovirus packaging components, gag and pol (Landau et al. (1992) J. Virol.:66). Plasmid number three contained vesicular stomatitis virus G glycoprotein (VSV-G).

The stability and target cell range were increased by VSV-G pseudotyping the retroviral vector (Burns et al. (1993) Proc. Natl. Acad. Sci. USA, 90; Yee et al. (1994) Proc. Natl. Acad. Sci. USA, :91). The lipid was used in a 1:3 ratio of total DNA:lipid with a final volume of 0.947 μl/cm² of Transit-LT1. The amount of each plasmid was 0.1053 1 μg/cm². After 4.5 to 7 hours incubation with the transfection reagents the cells were refed with complete growth media (Irvine Scientific). Approximately 48 hours later the supernatant (1000 mL) was collected and frozen at −80° C. Every 24 hours after that, the supernatant was harvested (1000 mL per day) and frozen for an additional four to five days.

Efficient retroviral production over this length of time has not been previously described for transient transfections. FIG. 15 shows an example of titer yields, represented as neomycin resistant colony forming units per milliliter. Each daily harvest yielded 1000 mL, resulting in up to 4×10⁸ viral particles per day, clearly enough to cover the ribozyme library complexity. Furthermore, when transfection efficiency of the Cf2A12 cells was evaluated with an EGFP-containing retroviral plasmid, >90% of the cells were EGFP positive.

On a cell factory scale, this amounts to approximately 2×10⁸ transiently transfected cells, each of which is producing different ribozyme-carrying retrovirus. This level of transfected cells ensures maintenance of the library complexity. Indeed, transfection efficiency is crucial to proper library production. Factors such as cell plating density, lipids used, DNA ratios, harvest times and volumes all have been optimized to assure high complexity libraries. As a final confirmation for the library, the produced retroviral library was subjected to RT/PCR to amplify the ribozyme insert and the pool was cycle sequenced. Random libraries gave equal intensity sequencing bands across all four lanes (G,A,T and C). Finally, this process was scaled up from 9.5 cm² to 6,320 cm² (cell factory, Nalge/Nunc) and could be scaled up to 85,000 cm² (cell cube, Corning/Costar).

### B) Clarification of Retroviral Vector Ribozyme Library

We have found that supernatant containing retroviral vector ribozyme library must be clarified or filtered prior to use. If the supernatant was not clarified it was generally too toxic to the target cells to achieve transduction of a library of high complexity. In addition the supernatant needs to be filtered if it will be concentrated.

First the supernatant was thawed at 37° C. Then it was placed into centrifuge tubes (Falcon) and spun at 270 G for 5 minutes at 4° C. The supernatant was poured off and pooled. The supernatant was then filtered through a 0.8 μm filter (Sartorius). If the titer level was high enough then this was suitable for use on target cells. If not, the material was further processed and concentrated. This filtration has been scaled up from 5.3 cm² to 61 cm² (2 ft²) and can be scaled up further to accommodate larger volumes.

### C) Concentration of Retroviral Vector Ribozyme Library

Once supernatant was collected over several days, spun down and filtered it was concentrated to increase the titer level. Using a smaller volume on target cells is ultimately better, the chance for toxicity is decreased and the multiplicity of infection (MOI) can be increased if necessary to achieve high complexity in the transduced cells. The method used for concentrating the retroviral vector was hollow fiber ultrafiltration (A/G Technology Corp.).

Clarified (filtered) supernatant was placed into a plastic bag with two ports (Nalge/Nunc). This was connected to the hollow fiber ultrafiltration system which has an ultrafiltration cartridge with a 500K nominal molecular weight cut-off. The supernatant is circulated through the system under constant pressure with a range of 6-11 psi on the inlet and 5-8 psi on the outlet. The system works by retaining the retroviral vector particles and filtering out smaller particles as well as fluids. During the process the system was stopped and back-flushed to avoid fouling the cartridge.

Once the volume was reduced 10- to 50-fold the process was stopped and the concentrated supernatant was back-flushed again, to increase recovery, for a volume equal to 50-75% of the final volume. The concentrated supernatant was then aliquoted and frozen at −80° C. An example of the clarification and concentration yields is shown below for a single timepoint harvest, resulting in recoveries of over 50% with nearly 20-fold concentration in titer. This process has been scaled up from 0.031 ft² to 0.7 ft² and can be scaled up to 5.2 ft² to accommodate larger volumes.

TABLE 6
Yield during clarification and concentration.

| | cfu/ml | TOTAL cfu | RECOVERY |
|---|---|---|---|
| CRUDE | 3.85 × 10³ | 1.8 × 10⁷ | 100% |
| CLARIFIED | 2.93 × 10³ | 1.4 × 10⁷ | 78% |
| CONCENTRATED | 6.57 × 10⁴ | 1.0 × 10⁷ | 52% |

### D) In vitro Transduction Assay of Retroviral Vector Ribozyme Library

Once retroviral vector ribozyme library is produced the level of transducibility needs to be quantitated and a titer value needs to be determined to verify that there is sufficient complexity to cover the ribozyme library. This enables each production (transfection, filtration and concentration) to be evaluated as well as the determination of multiplicity of infection (MOI) for target cells.

Human fibrosarcoma cells (HT1080) were seeded at 1.05×10⁴ cell/cm² in 6-well plates (Corning/Costar). Approximately twenty-four hours later they were refed with growth media (Gibco BRL) containing 6 μg/ml polybrene (Sigma). 10-fold dilutions of vector were made and placed on the cells. Approximately 24 hours later the cells were refed with growth media (Gibco BRL) containing 800 μg/ml Geneticin, G418 (Gibco BRL). The cells were refed two more times over the next eight days and 12 days after the transduction the cells were stained with coomassie blue stain. The colonies were then counted and the colony forming units per milliliter are determined. The transducibility of all cell types is not always the same as it is for HT1080 cells. Therefore, titer analysis was also performed using the target cells and the quantity of vector required for delivery of a full library complement was adjusted to reflect their specific transducibility.

### Example 7

### AAV Vector Ribozyme Library Production, Purification and Characterization

The protocol describes the exact way to make the aav vector library using a “cell factory” (cell factories, VRW, Cat.No.: 170009) large scale culture device.

### A) Seeding of the Cell Factory

You will need one autoclaved Q bottle: autoclave it at least for 45 min. and let it cool off before starting.

1) Prepare a suspension of 63200 A549 cells/ml. Since the Cell Factory holds 1 liter of media, to have enough cells you will need at least 10 confluent T225 before seeding. If possible, use freshly thawed cells, since they give a better production. Ideally, they should be around passage 5: do not use cells which have gone through more than 10-15 passages.

2) When calculating the number of cells needed, don't forget to include a check flask: you can use any size, but a T162 is ideal. This means that your final number of cells needed will be 63200 A549 cells multiplied by 1025 ml (1000 ml for the Cell Factory and 25 ml for the check flask). Leave some room for error (i.e. plan a little extra volume).

3) Trypsinize all of your flasks with 3 ml trypsin/flask. When the cells are trypsinized, add 3 ml of normal growth media to neutralize the trypsin.

4) Combine all of the cells into a 50 ml conical vial. Count the cells to find the volume needed to seed the Cell Factory+the check flask (usually, if the A549 are confluent, you will need about 25-30 ml out of the 50 ml conical vial): transfer this volume to a new 50 ml conical vial.

5) Spin the cells for 10 min. at 3000 rpm: this step is done to get rid of the trypsin which could interfere with the production.

6) You can now resuspend your cells into the media you will use to seed the Cell Factory. When working with A549, use DMEM+10% FBS+1×Sodium Pyruvate+1×Pen/Strep. (Pen/Strep. is not usually necessary, but contamination is common in the Cell Factories).

7) Mix the media+cells thoroughly, to assure even suspension.

8) Unpack the Cell Factory and the Q bottle (make sure it has been autoclaved properly!!). Place the Cell Factory standing on the short side which does not have the adapter caps, with the adapter caps facing you in the higher position. Pull out one of the adapter caps from the Cell Factory upper corner, and attach the Q bottle with the tube connector to the Cell Factory (the Q bottle will not attach if the adapter cap has not been properly removed). Detach the paper from the second adapter cap, which does not need to be removed: this provides ventilation, and the Cell Factory will not fill up if the paper has not been removed. Insert an air filter into the adapter cap which is not connected to the Q bottle.

9) Seed the check flask first (25 ml of media+cells into a T162): pour the remaining media+cells into the Q bottle. Since this step is tricky, and it is easy to spill, you can use a 100 ml pipette to transfer the cell suspension into the Q bottle, but this process is often laborious.

10) Make sure the Q bottle is properly attached to the Cell Factory. Swirl the contents of the bottle to assure even suspension. Tilt the Cell Factory on its side, so that the cell suspension will start to flow into the cell factory. Initially, the Cell Factory will not fill up evenly: after all of the cell suspension has entered the Cell Factory, wait a few seconds and watch as the cell suspension equilibrates (see picture #8, #9, #10).

11) When equilibrated (all levels must have the same amount of media in it), tilt the Cell Factory back up, with the adapter cap facing you as in the starting position. Detach the Q bottle, reattach the adapter cap and place the filters with the writing toward you into the adapter caps (listen for a snap when putting the filters in place). (See picture #11, #12).

12) Carry the Cell Factory and the check flask to the incubator. Do not tilt the Cell Factory toward the filters while carrying it!!! All the levels are connected, and if you tilt it toward the filters all of the cell suspension will flow to the bottom level (see picture #13). Make sure all levels are evenly covered with media, because often islands with no media can form.

13) Incubate at 37° C., 5% CO2 for 24 hours before transfecting.

14) Prepare your DNA for transfection (see 17)

### B) Transfection of the Cell Factory

You will need three autoclaved Q bottles: autoclave them for at least 45 min. and let them cool off before starting.

15) 24 hr. after seeding, the Cell Factory should be in between 60% and 80% confluent: this can be checked with the check flask. If the confluency is highly above or below this percentages, you might consider waiting another day (for confluency too low) or reseeding the Cell factory (for confluency too high).

16) Mix 6128 μl (6000 μl of Lipofectamine are needed for the Cell Factory and 128 μl are needed for the check Flask) of Lipofectamine with 249 ml of Optimem. Mix well and let the liposomes form by incubating at room temperature for 30 min. or longer.

17) Mix 667.1 μg (650 μg for the Cell Factory and 17.1 μg for the Check Flask) of each DNA (667.1 μg of library DNA plus 667.1 μg of Ad8 which is Cap and Rap expressing plasmid)with 225 ml of Optimem. Let the DNA distribute evenly in the Optimem by incubating at room temperature for 30 min. You will need the helper plasmid DNA (Ad8 or pAVAd) and the plasmid containing your gene of interest. Usually, Qiagen preps do not give you very high quality DNA so, in general, a Cesium Chloride prep might be a better choice. Nevertheless, I have achieved very good productions by using the Endo-free Qiagen Maxi or Giga prep and by precipitating the DNA with Ethanol before transfection (add 2.5 volumes of 100% Ethanol and 1/10 of a volume of Ammonium Acetate. Precipitate at −20° C., spin for 10 min. at 4° C., wash with ice cold 70% Ethanol and dry the pellet). Remember to keep the DNA sterile!! In order to achieve sterility, dry the DNA pellet in the hood and resuspend with Tissue culture grade water. Estimate the concentration of your resuspended DNA on a gel. Once the DNA is resuspended, take out a small aliquot for investigative digestions. I have been doing the following check digestions for Ad8 resulting in the following bands:

Ad8 check digestions and resulting bands:

Xba: 1 band, ˜3.5 kb. 1 band, ˜4.1 kb

EcoRV: 1 band, ˜7.8 kb

Xba+EcoRV: 1 band, ˜320 bp. 1 band, ˜3.1 kb. 1 band, ˜4.1 kb

PvuII: no bands

PvuII+EcoRV: 1 band, ˜7.8 kb Any Glycerol stock in my box labeled “Ad8” or “pAVAd” can be used for production.

18) Combine the DNAs+Optimem mix to the Lipofectamine+Optimem mix. Let the DNAs complex with the Liposomes by incubating at room temperature for 45 min. Right before transfecting, add the Ad5 (Adenovirus). We have been using 51 μl of the Adenovirus lot#042197 (lell titer). This stock is stored in the −80 C. Revco refrigerator in my rack. Mix well.

19) Empty the media from the check flask and transfect with 12.5 ml of the transfection mix. Empty the Cell Factory as seen in pictures #13, #14, #15 from all of the media. Attach a new sterile Q bottle and, after it is attached, pour the Lipofectamine+DNAs+Ad5+Optimem mix into the Q bottle. Fill up the Cell Factory with the transfection mix as before. The mix has a total volume of only 500 ml, so make sure it is evenly distributed onto all of the layers.

20) Bring the Cell Factory and the check flask back into the incubator and incubate it at 37 C., 5% CO₂ for at least 5.5 or 6 hours (If the transfection was done in the late afternoon, overnight incubation is possible, even though it put a slightly higher stress on the cells).

21) After the incubation, feed the Cell Factory with 500 ml of DMEM+20% FBS+1×Sodium Pyruvate+1×Penicillin/Streptomycin. Follow the same instructions as before. Feed the check flask with 12.5 ml of the same media.

22) Place the Cell Factory back into the incubator and incubate for 2 to 4 days. The incubation varies depending upon the titer of Ad5 used, but it should always stay on and never exceed this range. The cells can be checked for cytotoxic effect by examining the check flask. By day 2 the cells should start to swell up and they will be ready to be collected when 90-95% can be detached simply by banging the flask.

### C) Collection of the Cell Factory

23) Autoclave one plastic tube+tube connector attached on one side: this is basically the same tube which is connected to the Q bottle but without the Q bottle. This tubes can be found in the same cabinet of the Q bottles.

24) Take the Cell Factory out if the incubator and place several blue diapers on the bench: wear goggles and mask in case a breakage of the Cell Factory occurs. Hold the Cell Factory with both hands on its sides, parallel to the bench: bang the Cell Factory forcefully for about 30-40 times onto the bench. The media should become very cloudy and you should notice the cells detaching.

25) Bring the Cell Factory under the hood and empty the Cell Factory as before. This time, nevertheless, instead of the Q bottle, attach the tubing and place the exit end into the collection vessel of your choice. This vessel will have to hold at least one liter. Make sure the collection vessel will not topple over: either secure the vessel or make sure it is heave enough so it will not fall. Be Careful! The Cell Factory contains a lot of Adenovirus. From this point on you can follow the instructions in the “Purification Protocol”, starting directly with the microfluidizing step.

26) Store the collected cells and the supernatant containing AAV at 4 C. Bag the empty Cell Factory and throw it into the biohazard container.

### D) Production of rAAV Library

Recombinant adeno-associated vector library was produced by a transient transfection/infection process followed by a streamlined dual column chromatography method. A human lung carcinoma cell line, A549, was transfected using a cationic lipid, LipofectAmine (Life Technologies) at 0.5 μg /cm², to introduce 0.1 μg /cm² each of an AAV packaging plasmid and the ribozyme library plasmid. The AAV packaging plasmid encodes the wild type AAV rep and cap functions. The ribozyme library plasmid contains the ribozyme library sequences flanked by the AAV ITR (inverted terminal repeats) which provide the replication structures and encapsidation signal.

The A549 cells were simultaneously infected with human adenovirus type 5 at an MOI of 200 to supply helper functions for AAV encapsidation. After approximately 72 hours of transfection/infection, both the cells and supernatant were harvested and Benzonase (AIC) at 10 U/ml added to degrade non-packaged DNA. Collection and processing of the supernatant allows for the recovery of the approximately 30-70% of the vector shown to be in the supernatant fraction. The cells/supernatant mixture was microfluidized at 2000 psi to disrupt the intact cells, releasing any intracellular rAAV. The lysate was then incubated at 37° C. for 1 hour to allow the Benzonase to degrade DNA released during the cell disruption. The lysate was filtered through a 0.2μ polypropylene filter (Sartopure PP, Sartorius) to remove cell debris. The lysate was then loaded directly in the original physiological buffer onto a Poros BioCad high pressure liquid chromatography system (Perkin Elmer).

### E) Concentration and Purification of rAAV-βgal from Cell Lysate

Recombinant AAV vectors (rAAV) are generally obtained by harvesting and lysing vector producing cells. It has been reported by several groups, however, that much of the rAAV is released into the culture supernatant prior to cell harvesting, generating a loss in vector recovery. Estimates of the amount of rAAV present in the culture supernatant vary from 30-70%. This variability is most likely dependent on when cells are harvested following adenoviral infection. If the amount of rAAV present in culture supernatant is indeed significant (>50%), then it would be useful, from a production viewpoint, to recover this vector and minimize losses.

In order to produce clinical grade vector it will be necessary to purify the rAAV away from adenovirus as well as removing contaminating nucleic acids. Cellulofine sulfate column chromatography has been used for concentration of rAAV (Tamayose at al., 1996, Human Gene Therapy 7:507-513). However, a small amount of adenovirus as well as various serum and cellular proteins were always co-eluted with rAAV particles from the column. Anion exchange chromatography has been used to purify adenoviral vectors (Huyghe et al., 1996) and anionic resins are known to bind nucleic acids. Previous data indicates that rAAV will not bind to particular anion exchange resins (DEAE and HQ) under physiological salt conditions. Therefore, we developed a chromatography procedure to purify and concentrate rAAV from cell lysate by employing an anion exchange column (HQ) to “pre-clear” a lysate of adenovirus and nucleic acids followed by purifying rAAV with a cation exchange (SP) column.

The purification was performed in-line on dual columns without buffer or pH adjustment between columns to streamline the procedure, facilitating increased yield and eliminating potential contamination points. The first purification was through an anion exchange resin, i.e. Poros HQ (Perkin Elmer), followed immediately by purification through a cation exchange resin, i.e. Poros HS. The first column removes nucleic acids, residual proteins, and greater than seven logs of contaminating adenovirus. The second column concentrates the rAAV and removes additional protein contamination, resulting in removal of 99% of starting protein. Fractions eluted from the second column containing rAAV were pooled and formulated by the addition of MgCl₂ to stabilize the rAAV. The formulated vector was heated for 1 hour at 58° C. to remove any residual contaminating adenovirus. The formulated vector was stored at 4° C. A sample purification table of rAAV vector is shown in Table 7.

TABLE 7
Sample purification table of AAV vector.

| | | Total | Total | | | |
|---|---|---|---|---|---|---|
| | Volume | protein | rAAV | Spec. | | |
| Sample | (mL) | (mg) | (IU) | Activity* | % Yield | Fold Purif. |
| Crude | 2100 | 4830 | 9.9e8 | 2 | 100 | 1 |
| Purified | 10 | 15.6 | 1.5e9 | 961 | 152 | 481 |

The results indicate that in addition to removing 99% of the contaminating proteins, the tandem column purification scheme removes adenovirus as well. Therefore, by combining an SP cation exchange column with a tandem HQ anion exchange column we are able to produce highly-purified, adenovirus-free rAAV.

### F) Stability of rAAV Vectors

Various parameters affecting the stability of rAAV vectors were evaluated including storage buffers, storage temperatures, multiple freeze/thaw cycles, benzonase and RQ1 DNase. In summary, we have optimized each parameter resulting in highly stable rAAV vectors showing no significant loss of titers.

1) Multiple Freeze/Thaws.

rAAV-NGFR cell lysate was used that had already been frozen/thawed 6 times. Centrifuged (C) and uncentrifuged (U) lysate were frozen and thawed once (C1 and U1), twice (C2 and U2), and three times (C3 and U3) by setting them into the −80° C. for 1.5 hours and then quick thawing(by swirling) in a 37 C. water bath. HeLa cells were transduced with 20 μl and 80 μl of each sample and rAAV-NGFR activity was analyzed by FACS on Day 2. It appears that the rAAV vector can withstand up to 10 freeze/thaw steps stored as either centrifuged or uncentrifuged cell lysate.

2) Glycerol Storage Buffers.

The effects of 10% glycerol and 2% FBS/1% Glycerol on −80 C. storage of HPLC purified rAAV-NGFR were studied. Purified rAAV was resuspended in the appropriate buffer and stored at conditions indicated. The next day the whole viral suspension was transduced onto HeLa cells (1×10⁵ cells/well) and analyzed by FACS 48 hours later. The data indicate that rAAV is stable in both buffers (and maybe slightly more stable in the 10% Glycerol). rAAV also appears to be stable overnight at −80 C. in the buffer in which the vector is eluted off the HPLC.

3) +40° C. Cell Lysate Stability Studies.

The stability of the rAAV when stored at 4 degrees C. in unclarified lysate was studied. It appears that the vector is stable when stored at 4 degrees C. for at least 4 weeks. A similar study will be done with HPLC-purified rAAV vector.

4) Effect of Benzonase/RQ1 DNase Treatment on rAAV Vector Stability.

Since Benzonase and RQ1 DNase are adopted in our rAAV production scheme to degrade nucleic acid contaminants, effect of Benzonase or RQ1DNase on rAAV vector stability and infectivity was evaluated. rAAV-NGFR vector was treated with either Benzonase or DNase. To 100 μl of the vector was added: 1 μl 1M MgCl2 and 1 μl Benzonase(280 U/μl). To another 100 μl of the vector was added: 1 μl 1M MgCl2 and 1 μl RQ1 DNase (1 U/μl). These tubes were incubated at room temperature for 1 hour. Activity of Benzonase and RQ1 DNase at clearing the RNA in the lysate as well as most of the DNA were verified by gel electrophoresis. The samples were then diluted 1:10 and 10 and 100 μl of these dilutions were transduced onto HeLa's cells (10⁵ cells/well) and FACS on Day 2. The results show that neither Benzonase nor RQ1 DNase drastically affects rAAV-NGFR titer. Similar results were obtained when repeated with another vector, rAAV-Neo.

Using splinkerette PCR followed by southern blot analysis of the PCR products with radiolabelled AAV-specific probe, we have demonstrated integration of rAAV vector into the target cell chromosome with relatively high efficiency in two cell lines Molt 4/8 and CD 34+ primary human stem cells. Rather than revealing completely random integration, our data indicated that there are multiple “preferred” sites (hot spots) of rAAV integration.

### Example 8

### Rescue of Ribozyme Genes from Tissue Culture Cells

After application of the ribozyme library and selection of the desired phenotype, it is possible to “rescue” the responsible ribozyme(s) from the selected cells. The rescued ribozyme(s) are used both for re-application to fresh cells to verify ribozyme-dependent phenotype and for direct sequencing of the ribozyme to obtain the probe to be used for identifying the target gene.

In one approach, ribozyme genes may be rescued from tissue culture cells by either PCR of genomic DNA or by rescue of the viral genome (either AAV or RVV). To rescue by PCR, 2×10⁵ cells were lysed in 50 μL of lysis buffer (50 mM KCl, 10 mM Tris pH 9.0, 0.1% Triton X-100, 5 mM MgCl₂, 0.45% NP-40 and 200 μg/ml proteinase K) at 56° C. for 2 hours. The proteinase K was then inactivated by incubation at 95° C. for 5 minutes. The PCR reactions consisted of 25 μL cell lysate, 200 uM each dNTPs, 1×Taq Buffer II (Perkin-Elmer), 300 nM of each primer and 2.5 units of Taq DNA polymerase, in a final volume of 50 μL. PCR conditions were as follows: 95° C.×5 minutes followed by 35 cycles of 95° C.=30 seconds, 68° C.×30 seconds, 72° C.×30 seconds, followed by 72° C.×5 minutes. Choice of PCR primers depends on the starting library vector and are designed to amplify from 200 bp to 500 bp containing the ribozyme sequence. The amplified Ribozyme fragment was then gel purified (agarose or PAGE).

This PCR product can be used for direct sequencing (fmole Sequencing Kit, Promega) or digested with BamHI and MluI and re-cloned into one of the Ribozyme expression plasmids. This PCR rescue operation can be used to isolate not only single ribozyme from a clonal cell population, but it can also be used to rescue a pool of ribozyme present in a phenotypically-selected cell population. After the ribozyme are re-cloned, the resulting plasmids can be used directly for target cell transfection or for production of viral vector.

A simpler and more efficient method for ribozyme rescue involves “rescue” of the viral genome from the selected cells by providing all necessary viral helper functions. In the case of retroviral vectors, selected cells were transiently transfected with plasmids expressing the retroviral gag, pol and amphotropic (or VSV-G) envelope proteins. Over the course of several days, the stably expressed LTR transcript containing the ribozyme was packaged into new retroviral particles, which were then released into the culture supernatant.

In the case of AAV, selected cells were transfected with a plasmid expressing the AAV rep and cap proteins and co-infected with wild type adenovirus. Here the stably-integrated AAV genome was excised and re-packaged into new AAV particles. At the time of harvest, cells were lysed by three freeze/thaw cycles and the wild type adenovirus in the crude lysate was heat inactivated at 55° C. for 2 hours. The resulting virus-containing media (from either the retroviral or AAV rescue) is then used to directly transduce fresh target cells to both verify phenotype transfer and to subject them to additional rounds of phenotypic selection if necessary to enrich further for the phenotypic ribozymes.

Similar to the PCR method described above, viral rescue of ribozyme allows for rescue of either single ribozyme or “pools” of ribozyme from non-clonal populations.

### Example 9

### Use of Ribozyme Libraries to Identify Targets Involved in Cell Differentiation

Many model systems take the advantage of cell growth during the selection procedure to screen library to identify candidate genes. The selection procedure limits its application to the systems where there is no cell growth or division but differentiation as the end result of selection. To address this question, we set up a system to explore the possibility of using EBV library in a cell differentiation study. We took advantage of the fact that EBV library can be replicated by itself as a plasmid form in human cells under the selection pressure. Ribozyme sequences can therefore be rescued by transforming bacterial cells directly using cellular DNA from a few cells after the first round of selection of the library.

THP-1 is a example of a suspension cell line which has potential to differentiate into monocytes and attach to flasks. THP-1 was transfected with EBV library and EBVU5 control plasmid. Transfected suspension cells were observed for the presence of adherent cells. We observed about 100 to 500 cells adherent cells for every 5×10⁶ transfected THP-1 cells. After washing away of suspension cells from the adherent ones, we were able to rescue the ribozymes sequences by direct transformation of the DNA from attached cells since ribozymes exist as circular DNA in cells. After multiple rounds of selection by adhesion and rescue by transformation, we would be able to identify the ribozymes responsible for the phenotype change. This selection process can be applied to any suspension cell lines including neuronal origin, osteoblastic cell line, hematopoietic cells, and mesenchymal stem cells for the identification of genes involved in controlling cell differentiation.

### Example 10

### Identification of Unknown Genes Responsible for Cisplatin Sensitivity

This example describes a selection procedure for any cells which are sensitive to drugs (e.g., chemotherapy drugs), radiation, or other agents, for identification of genes. The method is exemplified using the cancer chemotherapeutic cisplatin.

Cisplatin as an antitumor agent has been shown to have a broad range of antitumor activity. Some ovarian carcinomas, however, are intrinsically cisplatin resistant and fail to respond to chemotherapy at all. Others develop “acquired” resistance with a two to four fold change in the sensitivity of the cells during the treatment.

The cytotoxic action of cisplatin on DNA has been well studied (Andrews et al. (1990) Cancer cells 2(2): 35-43). The interactions of this drug with other components of the cell are, however, less well understood. There is much interest in how cisplatin enters cells, how it is transformed and inactivated, and how the DNA damage is repaired. Thus, discovery and characterization of genes involved in the cisplatin resistance may elucidate the processes and speed the advancement of chemotherapy treatment of cancers that fail to respond to cisplatin.

Approximately 2×10⁷ of the 2008 cell line (ovarian cell line sensitive to cisplatin) and UMscc10b were transfected by 200 μg of pAAV6Clib described in Example 2(A) using lipofectamine. The transfection efficiencies were from 27% to 35% by eGFP expression.

After transfection, about 5.4×10⁶ to 7×10⁶ of cells (1.5 to 1.9 of library equivalent) containing library DNA were selected by multiple rounds of addition of cisplatin at increasing concentrations. Differential resistance to cisplatin were displayed between the control cells and cells transfected with the library after multiple rounds of selection (Table 8). Colonies derived from the library transfected cells growing out of high concentration of cisplatin were expanded.

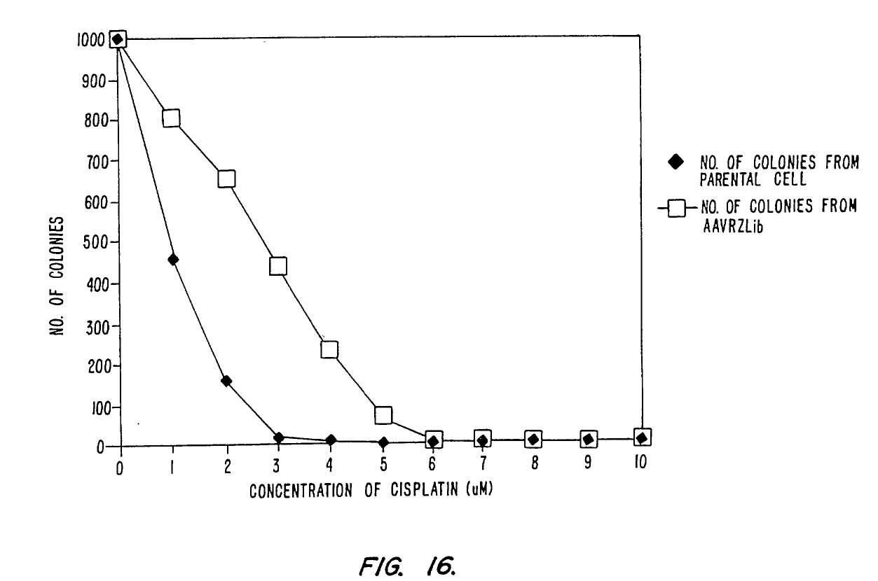

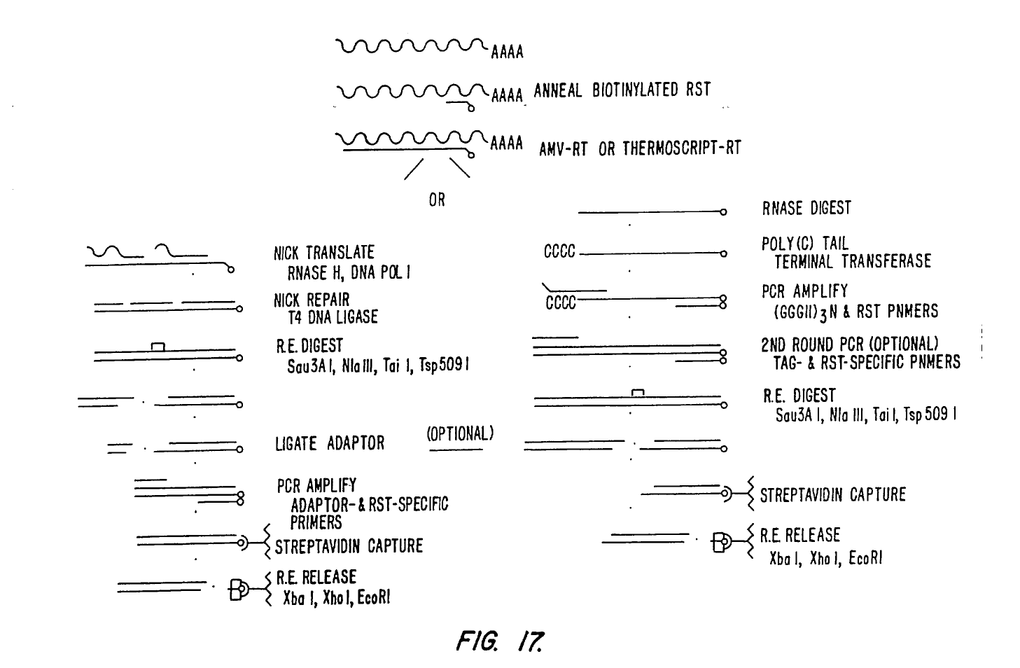

To confirm the library transfected cells after selection are indeed more resistant to cisplatin than either the vector transfected or parental 2008 cells, we compared the killing curve of the cisplatin of there three cell population. As shown in FIG. 16, library selected cells were much more resistant to the drug than the parental and vector transfected cells. Ribozyme sequences were rescued from the library transfected cells which were selected at high concentration were rescued by PCR by the method described in Example D and identified by sequencing. After confirming the function of ribozymes rescued from the resistant cells, we will identify genes controlling cisplatin sensitivity again based on the sequence of the ribozyme binding arms and GUC by the methods described in the next example.

TABLE 8
Differential resistance to cisplatin displayed between the control
cells and cells transfected with the library after
multiple rounds of selection.

| | % of surviving colonies | |
|---|---|---|
| Concentration of cisplatin uM | vector transfected | Library transfected |
| 0 | 100 | 100 |
| 4 | 2.14 | 18.4 |
| 5 | 0.97 | 10.3 |
| 6 | 0.79 | 5.2 |
| 7 | 0 | 2.3 |
| 8 | 0 | 0 |

### Example 11

### Identification of Genes Based on Ribozyme Sequence Tags (rsts)

### A) Identification of the Target mRNA Based on Ribozyme Sequence Tags by Genbank Searching

Ribozyme sequence tags (RSTs) can be identified by applying a ribozyme library to target cells and screening/selecting for desired phenotypic changes. After identification of RSTs that are responsible for the selected function, comparison of RSTs with known est sequences in the Genebank will identify known genes that can be potentially linked to the phenotype as described above. After ests have been identified, the location of the gene on chromosomes can also be discovered by searching the est sequence containing Aribozyme cleavage site against genomic database.

The genebank search, however, will not reveal any unknown genes that may contribute to functional change we are looking for. The following methods enable us to identify those cDNAs missing in the public databases.

### B) Identification of Target mRNA Based on Multiple Ribozyme Sequence Tags

The method described here identifies the relevant genes based on the sequence in formation of multiple ribozymes. Since each mRNA contains more than one ribozyme recognition site, several ribozymes that target same mRNA can be cloned from the cell population with the selected phenotype after library transduction or transfection. Based on the statistics that the possibility of two randomly picked ribozymes recognizing a same mRNA molecule is extremely rare (<10⁻⁶), if an mRNA molecule is recognized by two cloned ribozymes, the protein encoded by this mRNA molecule is likely to be responsible for the phenotype(s) (phenotypic character(s)) identified in the initial screen.

After multiple ribozymes have been identified to be responsible for the selected phenotype, primers will be designed to match the target sequence (sense sequences) of the ribozymes as well as the antisense sequences. For example, if the cloned ribozyme contains a sequence: 5′ AAAAUUUUagaaGCGG, (SEQ ID NO: _) where the underlined nucleotides indicate the regions of a ribozyme forming helixes with the target RNA, the primer that matches the sense sequence will be 5′ CCGCngtcAAAATTTT3′ (SEQ ID NO: _) and the one that matches the antisense sequence will be 5′ AAAATTTTGACnGCGG 3′.

The sense primers are used for a reverse transcription reaction to make the first strand of cDNA using mRNA isolated from the parental cells as templates. Then the sense primer used for reverse transcription reaction is paired with any one of the antisense primers except its own for PCR. If any two ribozymes recognize and cleave the same mRNA molecule, the fragment between primer 1 and primer 1R will be amplified.

For proof of principle, we designed sense and antisense primers according to the target mRNA sequences of eight ribozymes which have been cloned from the U138 cells that are selected for growing on soft agar after being transduced with the AAV-based ribozyme library as described herein. Then we used pairs of the sense and antisense primers to amplify the cDNA by RT-PCR using mRNA isolated from the parental U138 cells as template. The results showed that for certain primers, sequences of these DNA fragments will provide information on the proteins which are responsible for the phenotypic change.

### C) Isolation of cDNA from Ribozyme Sequence Tags (RSTs) Using Degenerated Primers and Poly dT Primer

The RSTs consisted of 15 to 16 ribonucleotides with one additional degenerate ribonucleotide at the 4th position from 5′ end. Such RSTs sequences are not good primers/probes for DNA PCR or southern hybridization assays that are normally employed for identification of full length cDNA from short DNA sequences. To circumvent the problem, we designed a degenerate primer based from the known RSTs (e.g., RRRR nGTC RRRRRRRNNNN 3′, SEQ. ID NO: _)

The last 4 randomized ribonucleotides at the 3′ end are used for efficient binding to the target template. The ribonucleotide R is determined by the individual RST; and nGTC corresponds to the cleavage site of ribozyme.

To identify mRNA which is cleaved by ribozymes in the selected cell population, the following PCR based method is utilized:

i) First round of PCR:

Poly A mRNA isolated from parental cells and the selected cells is used as templates. Reverse transcription PCR (RT-PCR) is performed using the polyT primer: 3′ NTTTTTTTTTTTT(20)CGAGGGTGAAGTCTAACCATTGT-5′ (SEQ ID NO: _)

ii) Second round of PCR:

RT-PCR product generated from the first round RT-PCR

RST primers and primer 3′ CGAGGGTGAAGTATAACCATTGT 5′ is used to specifically amplify cDNA containing RST sequences.

The PCR reaction will amplify target cDNA sequence from the ribozyme cleavage site to the end of polyA tail. Comparison of the amplification of mRNA from parental cells and the selected cells will allow us to determine which cDNA product is reduced from the selected cell population. Sequence analysis of PCR product will reveal information about the putative genes corresponding to RSTs. The full length cDNA can be readily isolated from the sequence information obtained.

### Example 12

### Identification of a Cellular Target gene Using a Biotinylated Ribozyme Sequence Tag

The isolation of one or more ribozymes from the library, based on their conferred phenotype, gives us a probe that can be used to clone the target gene. The probe sequence, or ribozyme sequence tag (RST), consists of 16 bases, 15 of which are specific for the target RNA. To illustrate the conversion from the sequence of an isolated ribozyme to an RST, an example of a ribozyme against PCNA mRNA is used. A ribozyme known to cleave PCNA mRNA has the sequence 5′-GAGCCCUGAGAAGGCG-3′, where the underlined bases are the arms of the ribozyme that bind to its target mRNA. An RST is the deduced sequence of the target mRNA, based on the complement of the binding arms of the identified ribozyme, including the requisite GUC required by the hairpin ribozyme. Thus, the RST corresponding to this ribozyme would be: 5′-CGCCNGUCCAGGGCUC-3′ (SEQ ID NO: _), where N=any of the four bases. Interestingly, previous knowledge of the hairpin ribozyme would have dictated that the N position could not be an A (Anderson et al, (1994) Nucl. Acid. Res: 22), however we have found that restriction to be incorrect and may be specific only for the native hairpin ribozyme. Therefore, an RST has, the following format: 5′-XXXXNGUCXXXXXXXX-3′ (SEQ ID NO: _), where X is a specific base (A, C, G or T) based on the complementary sequence of the isolated ribozyme and N is any of the four bases, thus resulting in 15 known bases and one N. This is sufficiently unique in the human genome for accurate target gene identification.

To clone the target gene, a specific oligonucleotide is synthesized containing the RST sequence (example below is RST for PCNA ribozyme), a few unique restriction sites (e.g. XbaI, XhoI, EcoRI) and a biotin molecule on the 5′ end (Table 9 below).

TABLE 9
Biotinylated RST Primer

```
XbaI XhoI EcoRI
                                                        | | | || |
          5′ -- Biotin-GCATG CTCCT CTAGA CTCGA GGAAT TCGAG CCCTG GACNA GGC -- 3′
                                                                                                        PCNA RST PRIMER
```

This oligonucleotide is used to specifically prime a reverse transcription (RT) reaction using target cell mRNA as the template (see FIG. 17). Following reverse transcription, second strand cDNA is made via nick translation (left part of FIG. 17). The resulting double-stranded DNA is digested with one of four restriction enzymes and a unique adaptor is ligated on (see Table 10 below).

TABLE 10
Adaptor & Adaptor-Specific Primer (underlined)

```
BamHI Sau3A I TaiI
                                                    | | | | | |
          5′ -- GCTAC AGCTC TCCGG ATCCA AGCTT GATCA TGACG TAATT CTGAG -- 3′
          3′ -- CGATG TCGAG AGGCC TAGGT TCGAA CTAGT ACTGC ATTAA GACTC -- 5′
                                                                  | | | | | |
                                                                  HindIII NlaIII Tsp509I
```

This restriction digest is necessary to make all RT products the same size (since we have no information about how far away the target gene 5′ mRNA end is away from the ribozyme binding site) and therefore make all future amplified PCR fragments the same size. Four different four basepair cutters (Sau3AI, NlaIII, TaiI and Tsp509I; each occurs on average every 256 basepairs) are included to assure that one of them is within ˜1000 basepairs of the RST, thus increasing the efficiency of PCR amplification. Only one restriction enzyme is used per reaction, and all four are tried independently if necessary to obtain specific target gene amplification. The adaptor contains a specific primer binding site which is then used to PCR amplify the target gene using the adaptor-specific primer and the RST primer (see FIG. 17). If there are background DNA bands following the final PCR, the specific target gene product is purified on streptavidin beads (Promega) followed by release with a restriction enzyme whose site is present in the RST primer. The resulting DNA is cloned into a plasmid for sequencing analysis and gene identification.

Occasionally, ribozymes are isolated that target low abundance mRNAs in the target cell. If the target mRNA is scarce enough, the single round of PCR amplification is insufficient to reproducibly detect the PCR product. In these instances, a second round of PCR can be included by adding a polyC tail to the 3′ end of the first strand cDNA (see right side of FIG. 17). This allows PCR amplification using a polyG primer (5′-GAAGA ATTCT CGAGG GGCCG CGGGI IGGGI IGGGI IGN-3′, (GGGII)3 Primer & Tag-Specific Primer (underlined) SEQ ID NO:_) and the RST primer prior to digestion and adaptor addition. The polyG stretch also contains inosine residues to prevent the non-specific priming observed when only G residues are used.

If further sensitivity is required, the polyG primer also contains a specific tag sequence on its 5′ end that can be used for a semi-nested round of PCR (again with the RST primer) to amplify the signal even further. In all cases, specific amplifications can be performed until the target gene product is visible on a gel and can be purified and cloned. Finally, since the cloned gene fragment still in not the complete cDNA, database searching is performed to identify the gene and if that is unsuccessful (i.e. the gene is completely unknown), this cloned gene fragment can be used as a highly specific probe to screen cDNA libraries to pull out the entire cDNA.

### Example 13

### Identification of Regulators of Gene Expression

### A) Transcription Regulators

A unique application of the ribozyme library is to identify transcriptional regulatory genes that up- and down-regulate specific gene expression. Transcription of mammalian promoters is a highly complex and tightly regulated event involving many cellular proteins interacting to affect the expression level of a gene. Regulation of genes such as oncogenes, tumor suppressors, cytokines, cholesterol pathway enzymes, globin genes, chloride channels, leptin and fat metabolism enzymes, etc. all play a role in various human pathologies. Currently, our knowledge of specific gene regulation is woefully incomplete. Using the ribozyme library, we are capable of identifying cellular factors that influence the expression levels of any protein for which the promoter is known.

To accomplish this, a reporter plasmid is created that contains the promoter of the gene of interest driving the expression of a reporter gene such as EGFP, antibiotic resistance or any other selectable marker. This reporter is stably introduced into an appropriate target cell. Application of the ribozyme library then allows introduction of specific Ribozyme that target transcriptional activators, resulting in a decrease in the reporter expression; and specific Ribozyme that target transcriptional repressors, leading to an increase in the reporter. Thus, by setting up the appropriate selection criteria for the reporter, we are able to use the Ribozyme library to identify both up- and down-regulators of the expression of a particular gene of interest. In fact, many cases allow us to select for both up- and down-regulators in the same cell population simply by altering our selection criteria. Furthermore, appropriate selection of cell type (specific or general) allows selection of both cell type specific regulators and general, ubiquitous regulators.

Identification of specific gene regulators clearly has therapeutic application. For example, identification of a transcriptional activator of a tumor suppressor gene could be used to screen for drugs that enhance the expression of the tumor suppressor in the appropriate cancer in vivo. Alternatively, gene delivery technology could be used to deliver the transcriptional activator gene itself. Less obvious is the fact that the selected ribozyme itself can have therapeutic value. If a ribozyme is isolated that targets a repressor of fetal hemoglobin, for example, the Ribozyme itself could be used to up-regulate normal globin expression in a patient with sickle cell anemia, where expression of sufficient normal globin (fetal or adult) is sufficient to correct the condition. Such a therapeutic approach could use synthetic, chemically stabilized Ribozyme, or Ribozyme genes delivered by gene therapy. Therefore, this technology allows us to ultimately control the expression level of any gene, with knowledge of the promoter being the only criterion. Below is a specific example of use of the Ribozyme library to identify genes involved in the regulation of the breast cancer susceptibility gene, BRCA-1.

### B) Post-transcriptional Regulators

Aside from transcriptional regulation, gene expression is also modulated by post-transcriptional events. These include mRNA processing, transport to the cytoplasm, mRNA stability, protein modifications and protein stability. Depending on the reporter system used, the ribozyme library can be used to identify genes in any of these regulatory pathways.

Genes that control the stability of mRNA, for instance, can be identified by linking the required cis elements with a reporter gene. For example, the 3′ untranslated region of the proto-oncogene c-fos is known to confer cellular instability to mRNAs. When linked to a selectable reporter, cellular factors that regulate this cis element can be identified by the Ribozyme library.

Protein stability also provides tight regulation for numerous gene products. Several cell cycle proteins, for example, contain PEST amino acid sequences that target the protein for rapid degradation. Adding PEST amino acids to a reporter (EGFP, for example) would allow identification of members of that protein degradation pathway. Another noteworthy example is the unidentified protease termed “aggrecanase”. In various bone/joint disorders, an unidentified protease is believed responsible for the breakdown of matrix proteins such as collagen. While the protease gene has not been identified, the amino acid recognition sequence susceptible to cleavage is known. By placing this amino acid sequence into a reporter protein, we can use the Ribozyme library to identify the protease gene(s) involved. This could lead to a therapeutic target for drug discovery in the treatment of arthritis, etc. Finally, it is well established that many human viruses utilize host cellular proteases to process their viral polyproteins (HIV and HCV are good examples). Again, the cellular genes are not yet identified however the protease recognition sequence is known and can be engineered into a reporter protein. Cellular proteases such as these, identified by the Ribozyme library, have tremendous therapeutic potential, both as targets for drug discovery and the ribozymes themselves as the therapeutic.

### Example 14

### Identification of BRCA-1 Gene Regulators

BRCA-1 is a tumor susceptibility gene for breast and ovarian cancer, which was cloned in 1994 (Miki et al. (1994) Science, 266). Mutations in this gene are thought to account for approximately 45% of families with significantly high breast cancer incidence and at least 80% of families with increased incidence of both breast and ovarian cancer (Id.). In contrast, only very few mutations have been found in sporadic breast and ovarian cancer (Futreal et al. (1994) Science, 266; Merajver et al. (1995), Nature Genet, 9). However, analysis of tissue samples from patients with sporadic breast cancer have shown, that BRCA-1 is expressed at diminished levels in sporadic breast cancer in these patients (Thompson et al. (1995) Nature Genet, 9). These data strongly suggest the presence of an altered upstream regulatory mechanism being responsible for the decreased expression level of BRCA-1.

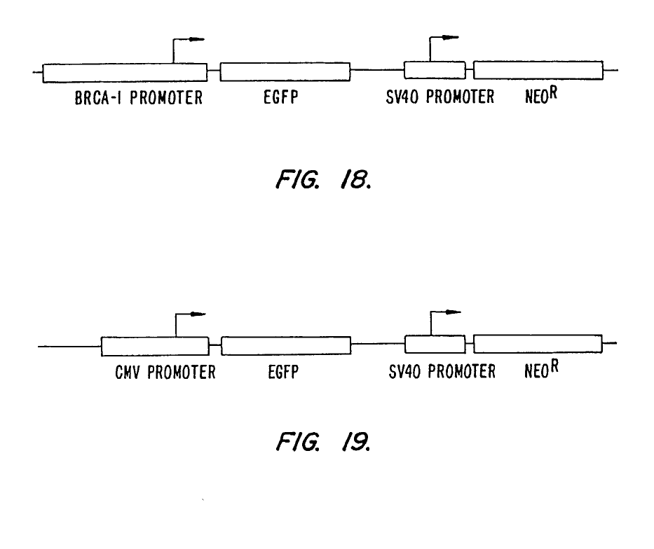

The application of the ribozyme library has the potential to identify such regulators. In order to receive a selectable screening system, the BRCA-1 promoter region was cloned in front of the selection marker EGFP (enhanced green fluorescent protein) as shown in FIG. 18.

As a positive control, the BRCA-1 promoter was replaced with the CMV promoter, thus allowing deregulated, constitutive EGFP expression (FIG. 19).

Both reporter constructs were stably expressed in established breast/ovarian cancer cell lines with high level (T47-D), medium to lower level (PA-1, MCF-7), or very low level (SK-BR-3) of endogenous BRCA-1 expression.

TABLE 11

| CELL LINE | BRCA-1 mRNA (pmol per ug total RNA) |
|---|---|
| SK-BR-3 | 6 |
| MCF-7 | 63 |
| PA-1 | 98 |
| T47-D | 125 |

By applying the ribozyme library to cells with different levels of endogenous BRCA-1 expression, positive as well as negative regulators of BRCA-1 can be identified. In general, this type of application allows the development of potential therapeutics directly in the form of ribozymes that suppress negative regulators of BRCA-1 expression or indirectly as gene therapy delivery of positive regulators of BRCA-1 for patients with sporadic breast or ovarian cancer.

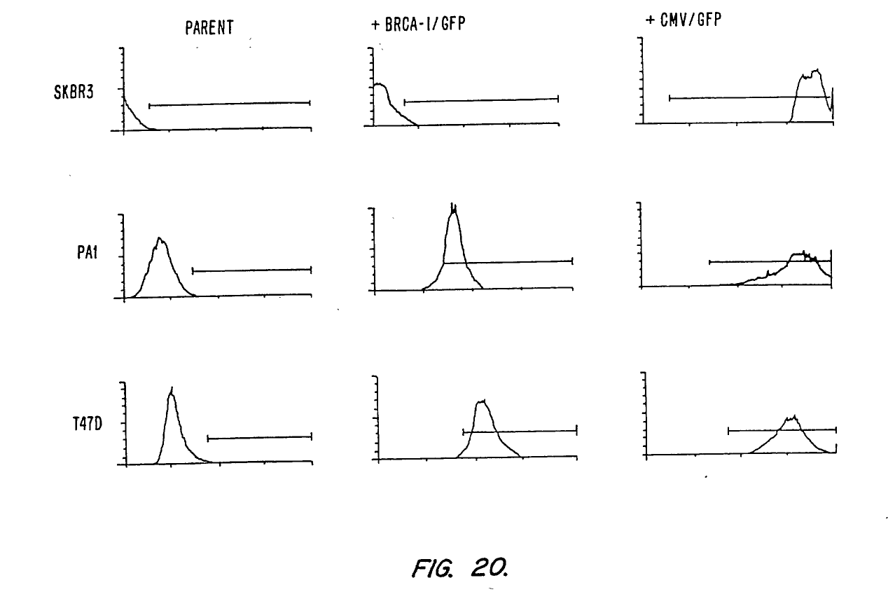

Single cell clones that are stably expressing the reporter construct were isolated from T47-D, PA-1 and SK-BR-3 cells. We have found that isolation of single cell clones greatly reduces the heterogeneity (and therefore the background) inherent in large polyclonal cell populations. In each cell type, the relative level of EGFP expression correlated with the level of endogenous BRCA-1 expression for each cell type, suggesting that the expression of EGFP is regulated by cellular factors working on the BRCA-1 promoter (see FIG. 20).

In comparison, the control reporter, in which EGFP is driven by the CMV promoter, revealed extremely high expression of EGFP, as would be expected from a strong viral promoter (+CMV/GFP in FIG. 20).

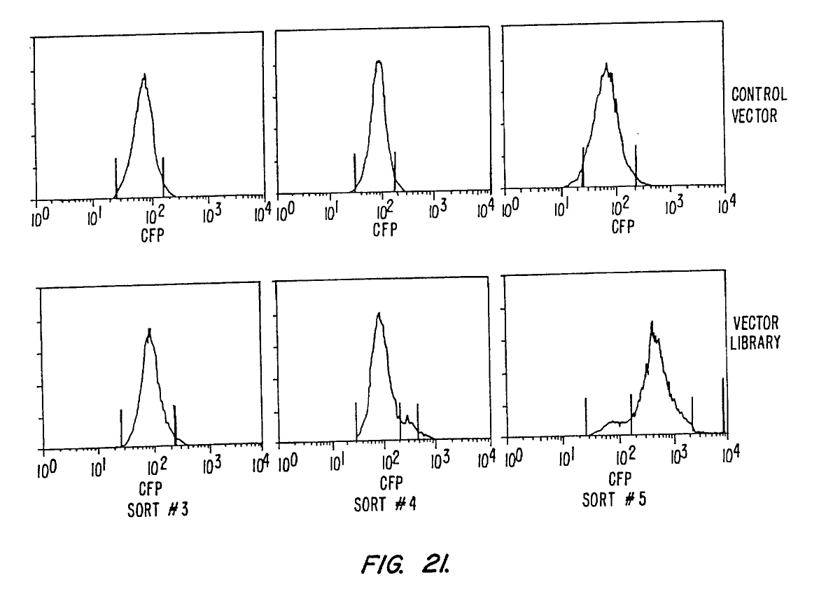

The retroviral ribozyme library was transduced into a BRCA-1 reporter cell clone and stably transduced cells were selected. In parallel, controls were transduced with retrovirus from a retroviral vector without a ribozymne expression cassette. Cells that were stably transduced with the ribozyme library or the control retrovirus and non-transduced cells were subsequently sorted by FACS (fluorescent activated cell sorting) for high expression of EGFP. After three rounds of sorts for the highest 10 percent (first round) or highest 3 percent (second and third round) of EGFP expressors out of the total population, an enrichment of approximately 10 percent was visible in ribozyme-transduced cells, while no enrichment could be detected in both controls (FIG. 21). After one further round of sorting for the highest 3 percent of the population, the majority of the population showed a higher expression level of EGFP, with a 15-fold increase in the EGFP mean fluorescence intensity, while the control populations remained unchanged.

Ribozymes responsible for this change in EGFP expression are rescued and the phenotype is verified by BRCA-1 western blotting and RNA analysis. Verified ribozyme sequences are used to identify the target gene(s) responsible for BRCA-1 regulation. In addition, Ribozyme such as these that result in the upregulation of BRCA-1 can be used as therapeutics for breast and ovarian carcinomas and possibly other tumor types.

While this example appears very clean, issues of background are of critical importance. For example, when we performed a similar FACS selection for low expressers of EGFP from the same PA-1 reporter cell, the Ribozyme library treated cells and the controls both gave a selected “low expression” population that was enriched in every successive sort (data not shown). Clearly, in this example, we were selecting for a sub-population of the cells that already had low EGFP expression, completely independent of the introduced ribozymes. To get around this background problem, three different routes are taken: 1) Several single cell clones are analyzed with the Ribozyme library in parallel, with the goal of identifying a particular cell clone that does not harbor this heterogeneity; 2) Include two different reporters in the same cell clone, for example BRCA promoter driving EGFP and BRCA promoter driving HSV tk. Any ribozyme that is truly affecting BRCA promoter regulators will affect both reporters, allow background to be easily removed; and 3) Following the first sort, rescue the ribozyme genes as a pool and reintroduce into fresh reporter cells, FACS select again, rescue again, FACS again, etc. each time enriching for the responsible ribozyme(s) while selecting out any background.

### Example 15

### Identification of Cellular Factors Involved in Viral IRES-mediated Translation

Several pathogenic viruses initiate the translation of their viral proteins via an internal ribosome entry site (IRES). Polio virus (picomaviruses) and hepatitis C virus (pestiviruses) are two noteworthy examples. It is clear, at least for polio virus, that IRES-dependent translation allows the virus to shut off all host cap-dependent translation thus converting all translation machinery to the viral RNA. Deletion and mutation studies have indicated that host cellular factors are required to initiate translation via the IRES, however these cellular factors have yet to be identified. Indeed, IRES from polio and HCV both can initiate protein translation in the absence of any viral proteins.

Human hepatitis C virus has a positive strand RNA genome that encodes the viral polyprotein. Immediately following infection, the incoming RNA genome must be translated to create the viral proteins required for viral replication. Translation of the genomic RNA is initiated by an IRES located within the 5′ untranslated region of the viral RNA. The IRES is essential for viral protein translation and therefore continued viral replication. The IRES is specific for HCV at the nucleic acid level however RNA folding analyses indicate that the overall structure of the IRES is shared by other viral RNAs such as the pestiviruses.

Most of the therapeutic strategies currently under evaluation involve attacking or blocking HCV replication by interfering with different viral components (viral helicase, protease, genome, etc.). These strategies, unfortunately, frequently fail due to the high mutation rate of HCV, which allows rapid generation of escape mutants. The IRES, however, is highly conserved (>95%) in all known strains of HCV, indicating that mutations in this region are not tolerated. Furthermore, the cellular factor(s) will not be as prone to mutational selective pressures as the virus. Identification of cellular factors required for IRES activity would yield an entirely novel field for anti-HCV therapeutics.

This example describes the use of the ribozyme library to identify cellular factors involved in HCV IRES-dependent translation with the ultimate goal of developing novel anti-HCV therapeutics.

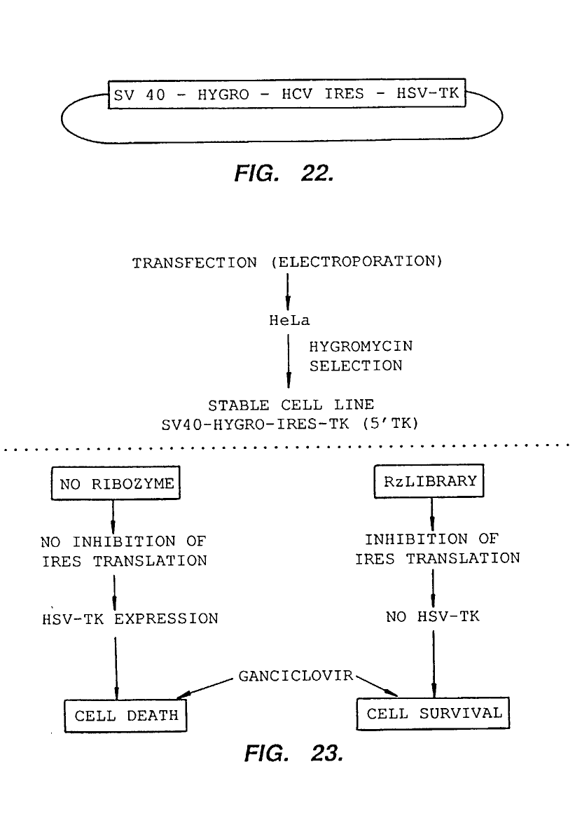

To allow selection for ribozymes that target IRES proteins, a reporter plasmid was constructed that contains the SV40 promoter driving expression of a bicistronic mRNA containing the coding sequence for hygromycin antibiotic resistance followed by the HCV IRES initiating translation of the HSV thymidine kinase (tk) coding sequence. While we use tk in this example, any selectable marker can be placed downstream from the IRES, such as EGFP, antibiotic resistance and cell surface markers. The vector was constructed such that the translation start site AUG for the tk reporter is the bona fide HCV core protein translation start site, thus assuring proper IRES-mediated translation (FIG. 22).

For gene identification, this reporter is stably transfected into HeLa cells (where HCV IRES activity has already been documented using hygromycin selection (FIG. 23). This “parental” cell population is called 5′TK.

Expression of the tk gene in these cells via the IRES confers sensitivity to the toxic effects of gancyclovir. Introduction of a ribozyme that inhibits expression of a cellular factor involved in IRES translation would result in a loss of tk expression and this cell would become gancyclovir resistant (see FIG. 23). As a control, a similar reporter plasmid was constructed without the IRES (or with a non-functioning mutant IRES), to verify that tk expression is IRES-dependent. To facilitate selection of the correct ribozyme from the library, it was necessary to start with a concentration of gancyclovir that effectively kills all parental cells, thus assuring a low background of false positives. To this end, 7.5×10⁵ 5′TK cells were exposed to various concentrations of gancyclovir and the number of surviving cell colonies is shown below. Due to anticipated heterogeneity in the original parental population, individual cell clones of the parental were also isolated and gancyclovir sensitivity was assayed as shown below in Table 12.

TABLE 12
Gancyclovir concentration (μM)

| | 4 | 8 | 12 | 16 | 20 | 40 | 60 | 80 | 100 |
|---|---|---|---|---|---|---|---|---|---|
| Parental | 109 | 38 | 17 | 15 | 9 | 7 | 2 | 2 | 1 |
| Clone 1 | nd | nd | nd | nd | 20 | 4 | nd | 0 | nd |
| Clone 2 | nd | nd | nd | nd | 0 | 0 | nd | 0 | nd |
| Clone 3 | nd | nd | nd | nd | 0 | 0 | nd | 0 | nd |
| Clone 4 | nd | nd | nd | nd | TMTC | TMTC | nd | 60 | nd |
| Clone 5 | nd | nd | nd | nd | 0 | 0 | nd | 0 | nd |

nd = not determined
TMTC = too many colonies to count

These data suggested that the original parental population was too heterogeneous, resulting in unacceptably high backgrounds. Three of the individual clones (#2, 3 and 5), however, were highly sensitive to gancyclovir as low as 20 μM. Ribozyme library is then introduced into these cell clones and gancyclovir selection is initiated. Ribozyme-expressing cells that survive under 20 μM are then harvested and Rz are rescued to identify cellular genes involved in IRES-mediated translation.

### Example 16

### Identification of Genes Involved in TRAIL-induced Apoptosis

Apoptosis, or programmed cell death, is a complex process by which cells can commit suicide when they receive the proper signals from either an external or internal source (Hetts (1998) JAMA. 279:300-307). One external induction mechanism involves cell surface proteins termed “death receptors” (Baker et al. (1996) Oncogene. 12:1-9). There are several such receptors (e.g. Fas, TNF-α receptor and TRAIL receptor) all of which contain a homologous intracellular region called a death domain.

When any of these death receptors are bound by their respective ligands, they initiate a complex signaling cascade which eventually leads to a disruption in mitochondrial integrity, fragmentation of chromosomes, nuclear condensation and cell shrinkage. Many of these same pathways are also involved in the programmed cell death of cells which have received apoptotic signals from within. For example, when c-myc gene expression is deregulated and constitutively activated, cells will undergo apoptosis in conditions, such as serum starvation or glucose deprivation, that are not optimal for growth (Evan et al. (1992)Cell. 69:119-128; Shim et al. (1998) Proc Natl Acad Sci U S A. 95:1511-1516). Understanding the apoptotic pathways is a very active area of research, with far-reaching applications from developmental biology to cancer and HIV therapeutics. Many genes which encode key players in the process have been identified. However, due to the complexity of the apoptotic processes, there are still many genes which encode components of the pathway which have yet to be identified. The ribozyme library will be used to identify

Selection of ribozymes (from the Rz library) capable of blocking TRAIL-induced apoptosis was investigated in the melanoma cell line G-361 (ATCC #CRL-1424). To determine their initial sensitivity to TRAIL, G-361 cells were plated at a density of 1×10⁴ cells/cm². Recombinant TRAIL (Alexis Biochemicals) was applied at concentrations between 10 and 200 ng/ml for anywhere from 16 hours to 2 days. The most efficient killing was found 2 days after adding 200 ng/ml TRAIL, however, this did not result in 100% killing.

To identify genes in this pathway, G-361 cells are stably transduced with the ribozyme library and then treated with 200 ng/ml of recombinant TRAIL. After two days of treatment, the TRAIL is removed and the cells are allowed to grow. Ribozymes that block apoptosis, and thus confer resistance to TRAIL, will allow that cell to proliferate. Ribozymes from these resistant cells are rescued, reintroduced to fresh G-361 cells and exposed to TRAIL again. This is to ensure removal of any ribozyme-independent, background resistance to TRAIL.

Since the conditions of TRAIL treatment does not lead to 100% cell killing, the isolation of the correct ribozyme(s) requires multiple rounds of rescue and reselection to enrich for the active ribozyme. After another round of TRAIL treatment, the selected ribozymes are rescued and reintroduced into G-361 cells again. After each round, the pool rescued ribozymes becomes enriched for ribozymes that interfere with TRAIL-induced apoptosis. Once the cycles of treatment and rescue result in a few different ribozymes, the sequence of the rescued ribozymes is then determined. These ribozymes can then be individually reintroduced into G-361 cells to verify their ability to interfere with TRAIL-induced apoptosis. The ribozyme sequences are then used to identify genes involved in the apoptotic processes.

### Example 17

### Identifying Genes in Cellular Differentiation Pathways

Beginning in the embryonic stage and continuing throughout the lifespan of an organism, cellular differentiation is required for the creation of all specific cell types in the body. In response to extracellular signals, pluripotent stem cells differentiate into terminally differentiated cells exhibiting specific functions and characteristics. Differentiation of nerve cells, muscle cells and cells of the immune system are just a few noteworthy examples. The genetic and biochemical pathways involved in these differentiation processes are extremely complex and little understood. Identifying genes involved in differentiation not only allows therapeutic control over the creation of specific cell types, but it also allows insight into the mechanisms controlling cancer formation out of specific cell types.

In many cases, cellular differentiation can be carried out in tissue culture. And in all cases, the differentiated cells exhibit one or more phenotypes that differ from the parental stem cell, thus allowing ready separation of differentiated and non-differentiated cells. Such selectable phenotypes include changes in cell growth/proliferation, changes in surface proteins (sort by FACS), loss or gain of adherence/differential trypsinization, changes in cell size (sort by FACS), etc. These conditions are well suited for application of the ribozyme library to select for blockage of differentiation and thus identify genes involved in any given differentiation pathway.

### Example 18

### Identification of Genes Involved in Neuronal Differentiation

Neuronal differentiation pathway is one of many examples that can be investigated using the ribozyme library strategy. Knowledge of its key players is important for understanding neurologic diseases and neuronal regeneration for potential disease therapeutics. There are many systems where neuronal precursors differentiate under certain growth conditions and form neuron or neuron-like cells. The completely differentiated neurons become post-mitotic and stop dividing. When the ribozyme library strategy is applied to these systems, the cells that do not enter post-mitotic state due to a specific ribozyme(s) will continue to grow and can be readily isolated. Rat pheochromocytoma PC12 cell line is one of the experimental neuron differentiation systems (Greene and Tischler, Proc. Natl. Acad. Sci. 1976) as are the human embryonic cell line NT2 that differentiates in response to retinoic acid (Andrews et al. (1984) Lab. Invest.50; Andrews et al, (1987) Development Biol: 103; Pleasure et al. (1993) J. Neurosci Res:35; Pleasure et al. (1992) J. Neurosci:12)

The strategy of using hairpin ribozyme library carried by retroviral vectors to investigate neuron differentiation on PC 12 cells is described. 2×10⁷ PC12 cells were seeded on five 150 mm collagen-coated plates on day 0, and cultured overnight in the growth media. The concentrated retroviral vectors containing ribozyme library of the full-complexity are used to transduce PC12 cells on day 1 at MOI of 2 for two hours. On day 2, an antibiotic selection drug (e.g. G418 at 500 μg per ml or puromycin at 1 μg/ml) is added to the culture to select for cells that received ribozyme vector. Media is changed on day 4 with growth media containing nerve growth factor (NGF) at 100 ng per ml (Boehringer Mannheim). The media is changed every three days with growth media containing NGF and antibiotic. Once neuronal differentiation is complete, only cells expressing ribozymes that block differentiation will continue to proliferate. These outgrowing cell populations are combined for ribozyme rescue. The rescued ribozymes (individual ribozymes or a pool of ribozymes) are then re-introduced into fresh PC12 cells exactly as the above. As this cycle of ribozyme application and selection is repeated, the resulting pool of ribozymes is enriched for ones that block neuronal differentiation. These enriched ribozymes are used to identify genes in neuronal differentiation.

Unfortunately, it is difficult to achieve 100% neuronal differentiation using PC12 cells, thus yielding high levels of false positive ribozymes. Thus, either multiple rounds of rescue and reselection are required, or we must find alternate ways of achieving terminal differentiation. Another alternative is to link neuronal differentiation with apoptosis. Following NGF treatment of PC12, if both the NGF and the serum are withdrawn, the cells go through apoptosis (Haviv et al, (1997) J. Neurosci. Res.:50). Untreated cells are not apoptotic after serum withdrawal. Thus, Rz that block the NGF pathways would also prevent any apoptosis in the absence of serum.

### Example 19

### Identification of Cellular Genes Involved in Vpr-mediated Cell Cycle Arrest and HIV Infection

Another application of the ribozyme libraries of this invention is to investigate the pathway of HIV-1 Vpr function. Vpr is an accessory viral protein, and has been implicated in several aspects of viral function as well as viral pathogenicity. However, the true role of the Vpr in the biology of the virus is not completely understood. Vpr causes cell growth arrest at G2/M (Levy et al, (1993) Cell 72:541, Rogel et al. (1995) J. Virol. 69:882, Jowett et al. (1995) J. Virol. 69:6304), but the mechanism and cellular factors involved have yet to be determined.

The investigation of this pathway is not only important for understanding HIV biology and pathology, but also for potential drug development against the virus. Due to the association of Vpr with the cell cycle machinery, this study may also have implications in understanding cancer or in cancer therapy. Since expression of Vpr prohibits cell proliferation, ribozyme-mediated knockout of a gene involved in the Vpr pathway results in a proliferating cell and thus a positive phenotypic selection. The unknown gene of interest is identified based on the ribozyme sequences. Similarly, other viral mechanisms involving cellular pathways could also be investigated, where ribozyme-dependent gene knockout results in resistance to infection and/or viral replication.

To accomplish this, 2×10⁷ HeLa cells were plated in ten 150 mm plate on day 0. The cells were co-transduced with retroviral vector ribozyme library of full complexity and a retrovirus carrying the Vpr-IRES-EGFP cassette, with MOI of 1 for each vector on day 1. Media was changed on day 2. On day 3, the cells were harvested by trypsinization and sorted for EGFP expression. The sorted cells are returned to tissue culture dishes. The cell colonies formed after day 15 are harvested by trypsin, and are FACS sorted again for EGFP, and returned to culture. Cells that continue to proliferate in the presence of vpr are used to rescue the responsible ribozymes. Re-introduction of these rescued Rz back into fresh HeLa cells in the presence of vpr allows verification of the Rz-dependent phenotype. The sequence of these positive ribozymes are then used to identify cellular genes that interact with or are downstream of vpr activity.

### Example 20

### Identification of Tumor Suppressors

As our understanding of cancer biology expands, it is becoming increasingly clear that tumor suppressors play as important a role in tumorigenesis as oncogenes. Loss of tumor suppressor genes, either by mutation, deletion or down-regulation, is often a key indicator to cancer susceptibility. Therefore, identification of novel tumor suppressor genes and generation of specific probes against them will enhance the future diagnosis and treatment of human cancers. Below is a list of several examples of using the ribozyme vector library to identify and clone tumor suppressor genes.

### A) Hela Revertant

Following exposure to the mutagen EMF, two stable HeLa (cervical carcinoma) clones were isolated that had lost all transforming properties (Zarbl et al, 1987, Cell:51; Boylan et al, 1996, Cell Growth and Diff:7). Along with the loss of their transformed morphology, these two clones (HA and HF) have lost their anchorage independence (i.e. no longer grow in soft agar or in suspension culture). Furthermore, their tumorigenicity in nude mice was completely abolished. Activation of a tumor suppressor in these revertant cell clones was indicated by the fact that cell fusions with original transformed HeLa resulted in loss of the transformed phenotype, a hallmark of a dominant tumor suppressor. This system is ideally suited for the use of the Rz library to clone this tumor suppressor because: 1) the system has very little background (i.e. cloning efficiency in soft agar is 0.05% for HF compared with 20% for the parental HeLa; and 2) due to the procedure by which these cell clones were created, it is most likely that only one gene has been activated in the revertant.

To identify the tumor suppressor, the retroviral ribozyme library was stably introduced into 2×10⁷ HF cells. As a negative control, HF cells were stably transduced with a retroviral vector carrying a non-specific Rz against HCV (also called CNR3). To isolate single cells that had lost the tumor suppressing phenotype, cells were plated into soft agar containing MEM and 10% FBS at a cell density of 4×10³ cells per cm². Both serum concentration and cell plating density was found to be critical at reducing background soft agar colonies in the negative control. It became evident that cell-cell proximity enhanced soft agar formation even in the negative control, thus lower cell densities equated to high selection stringencies. After two to three weeks in culture, several Rz library treated cells were exhibiting growth over the controls.

Fifty soft agar colonies from the Rz library treated cells were picked, along with 20 colonies from the negative control, and these pools of colonies were re-plated into fresh soft agar at higher stringency (1.5×10³ cells/cm²). Following 2 to 3 weeks culture, a 300-fold increase in soft agar plating efficiency was observed with the ribozyme library cells, compared with <2.5-fold increase in the controls (see Table 13).

TABLE 13

| | Primary Selection | Secondary Selection |
|---|---|---|
| Cells | (Colonies/10⁵) | (colonies 10⁵) |
| Hela | 50,000 | 50,000 |
| HF (revertant) | 10 | 25 |
| HF-Control Ribozyme | 20 | <50 |
| HF-Ribozyme Library | 59 | 15,000 |

Ribozyme genes from this secondary selection were rescued as a pool and reapplied to fresh HF cells to verify phenotype. Rz genes that confer soft agar growth from these rescue experiments are isolated and the anti-tumorigenic gene(s) that they target are identified.

### B) NIH 3T3

NIH 3T3 cells are an ideal system for identifying tumor suppressors because the cells are immortalized (suggesting that they have already incurred their “first hit” out of two required for transformation) but their tumorigenicity in mice is low to non-existent. Thus, inactivation of a tumor suppressor would yield transformation. In addition to growth in soft agar, transformed 3T3 readily form foci (anchorage independent colonies of cells) that grow up from the normal monolayer of cells in tissue culture dishes. While 3T3 cells are of murine origin, identification of a mouse tumor suppressor would easily lead to the identification of the human homologue using standard molecular biology techniques.

2×10⁷ NIH 3T3 cells were stably transduced with the retroviral ribozyme library and the cultures were allowed to reach confluence. Numerable foci were detectable in the Rz library treated population with very few in the negative controls of normal 3T3 nor mock transduced.

Foci from these populations were isolated by gently dislodging them from the plates, trypsinized to disaggregate and then replated on fresh dishes. After just several days in culture, tremendous numbers of large foci were formed in the replating of the Rz library transduced cells. This was observed prior to formation of the monolayer, suggesting a highly transformed population. In parallel, the replated control foci simply formed a normal monolayer without any increase in the few background foci.

Individual foci (as well as pools of foci) were picked and the Rz genes were rescued for re-application to fresh 3T3 cells to verify phenotype. Responsible Rz genes that confer the transformed phenotype are then cloned and their target genes are identified.

### C) Tumor Suppressors on Chromosome 6 and 11

Loss of heterozygosity (LOH) on human chromosomes 6 and 11 has been frequently observed in many human cancers and both chromosomes are believed to contain one or more important tumor suppressor genes (Robertson et al, 1996, Cancer Res:56, issues 7 and 19). Indeed, on chromosome 11 alone, at least 3 different regions of LOH have been identified in cancers such as breast, prostate, lung, ovarian, cervix, melanomas and neuroblastomas. Further evidence indicates the presence of tumor suppressors on these chromosomes since re-introduction of a wild type chromosome into LOH-transformed cells leads to suppression of in vitro growth and in vivo tumorigenicity (Robertson et al, 1996, Cancer Res:56, issues 7 and 19). Despite this knowledge, and a tremendous amount of scientific effort, identification of these tumor suppressor genes has remained elusive. Application of the Rz library to cells in which the wild type chromosome has been re-introduced is ideal for the identification of these tumor suppressors since Rz-dependent knockdown of the gene(s) would result in the return of the transformed phenotype.

Similar to the Hela system described above, chr6 or chr11 LOH melanomas easily form colonies in soft agar. However once the wild type chromosome is re-introduced (6n=chr6, 11n=chr11), the number and size of soft agar colonies is greatly diminished relative to the parental melanoma (see below). Again, serum concentrations and cell densities were critical in keeping the background formation of colonies in the 6n and 11n cells at a minimum.

To identify the tumor suppressors, retroviral Rz library was stably introduced into the melanoma cell line where chromosome 11 had previously been re-introduced (called (11n)4 cells). When chr11 was re-introduced into the parental melanoma to create the (11n)4 cells, it was linked in cis to the neomycin resistance gene. Therefore, the retroviral library used in these experiments was the pLPR library carrying only puromycin resistance, thus allowing stable selection for ribozyme using puromycin selection and maintenance of chr11 using neomycin selection. Rz transduced cells were then plated into soft agar at high stringency (low serum, low cell density) and the number of resulting colonies are shown below in Table 14.

TABLE 14

| Cells | Soft Agar Colonies |
|---|---|
| Parental melanoma | >1000 |
| 11n (parent + chrom 11) | <10 |
| 11n + control ribozyme | <10 |
| 11n + ribozyme library | 180 |

Increased soft agar growth following introduction of the ribozyme library suggests the presence of ribozyme capable of inactivating the tumor suppressor gene(s) on chr11. Individual ribozyme (and pool of Ribozyme) were rescued from these soft agar colonies and reintroduced into fresh 11n cells to verify the transfer of phenotype. Ribozyme genes that are isolated from this rescue are then used to identify the target tumor suppressor gene(s) active on chromosome 11. Further, while the data in this example focuses on chromosome 11, similar experiments are underway to identify tumor suppressor genes on chromosome 6.

### Example 21

### Identification of Unknown Genes Responsible for Tumor Suppression

Identification of an unknown gene responsible for tumorigenesis is accomplished by transducing any partially transformed cell line lacking the properties of tumors such as colony formation in soft agar and non tumorigenic in nude mice with ribozyme library. U138 MG cell line is an example. This cell lines was derived from a patient with a grade III anaplastic astrocytoma. This cell line exhibits noneoplastic features and no tumor growth in nude mice (D D Bigner et al, J Neuropathol Exptl Neurol 40:201-227, 1981).

1×10⁷ U138 cells were transduced with either AAV library (Example A) or with another AAV ribozyme library prepared as described in Example B with 50 to 60% transduction efficiency to introduce about 1.5 library equivalent virion into cells. The soft agar clonogenic assay was used as a measure of the tumorigenicity of ribozyme transduced cells. It is important to optimize the soft agar assay condition so that no colonies grow from parental or vector transduced cells but library transduced cell do grow in soft agar.

Cell number, serum concentration, and soft agar concentration can be varied to achieve the optimal condition for identify ribozymes responsible for the phenotype change. We optimized our soft agar culture condition as following: 0.6% agar in Eagle's minimal essential medium with 10% fetal bovine serum, penicillin-streptomycin, sodium pyruvate (1 mM) and non essential amino acid is first laid on a 100 mm tissue culture dish, 1×10⁵ cells (or on a 60 mm tissue culture dish, 5e4 cells) were resuspended in 0.35% agar dissolved in the same culture medium are plated on the top of 0.6% agar.

After transduction with the library and vector rAAV, U138 cells were plated on 100 mm tissue culture dishes. Three weeks after plating, library transduced cells grow into colonies while no colonies were generated by parental or vector transduced cells. The phenotype can be repeated each time we introduced library in U138 cells with frequency of 0 to 10 per 1×10⁵ transduced cells. The colonies were picked, expanded, resuspended, and replated back in the second round soft agar in the same conditions.

We observed that cells expanded from the colonies isolated from the primary soft agar plates indeed showed the change of phenotype to anchorage independent growth with much higher plating efficiency in soft agar. To confirm that these cells are more inmotorized, we compared the growth rates of two library selected cells isolated from soft agar colonies to the rate of the parental U138 cells. These two selected cell populations grew much faster than the parental cells. The cells which displayed anchorage independent growth and faster growth rates were investigated for the ribozyme sequences by both PCR rescue and by viral rescue.

Ribozyme sequences can be rescued by adenovirus in the presence of Rap and Cap expressing vector and by wild-type of AAV. Without extensive optimization of the rescue conditions, we got low efficiency of rescue by adenovirus and by wild-type AAV as many other research groups did. Thus, we rescued ribozyme sequences by PCR amplification using primers franking the ribozyme expressing cassette: 5′ PA (5′ CCGTTGGTTTCCGTAGTGTAGTGG 3′) and 3′ PA (5′ GCATTCTAGTTGTGGTTTGTCC 3′). The PCR condition is 94° C. for 2 min followed by 30 cycles of 94° C. for 30″, 56° C. for 30″, and 68° C. for 45″ then 68° C. for 7′ using the expanded long enzymes (BMB) according to the procedure recommended by the manufacture. The PCR products were cloned and sequenced. We have obtained 8 ribozyme sequences from colonies after the first round and second round of replating. To confirm inactivation of tumor suppressor gene expression by their cleavage activity, the individual ribozymes as well as their corresponding disable ribozymes and the control vector were introduced back into the parental U138 cells.

Ribozyme G1 isolated from library leads to the growth of colonies in soft agar. After confirming the correlation between ribozymes and the phenotype change of cells, the ribozyme sequences are used to determine the ribozyme sequence tag (RST). For example: RST sequence 5′ GCCA ngtc CCGGGTT 3′ is derived from ribozyme sequence 5′ AACCCGGagaaTGGC 3′. Gene sequences can be identified by genebank search or by methods described in Example G using RST sequences. Three of eight RSTs identified from U138 cells were mapped to a single chromosomal band at which loss of homozygosity are frequently associated with cancers of pancreatic (80%), prostate (30-75), head and neck (67%), colon (60%), ovarian (50-73%, breast (20-80%, renal (64%), and oral SCC (56%). The soft agar clonogenic assay can be applied to any partially transformed cell line which does not grow in soft agar under optimized conditions for the identification of tumor suppressors. For cell lines which have background colonies in soft agar, we can enrich the candidate ribozymes from the library by rescue ribozymes from pooled soft agar colonies by PCR, clone the PCR products in AAV vectors by shotgun cloning and transduction of AAV DNA isolated form pooled bacterial clones for multiple cycles of selection and rescue.

### Example 22

### IL-1b Knockdown in THP-1 Cells

Interleukin 1 beta (IL-1β) is an inflammatory cytokine produced by a variety of cells of the hematopoietic lineage in response to certain stimulatory factors. THP-1 cells are of monocytic derivation and produce significant amounts of IL-1β when exposed to lipopolysaccharide (LPS). To assess the efficacy of ribozyme knockdown of IL-1β, we generated 10 ribozyme constructs directed against the IL-1β gene, transduced the constructs into THP-1 cells using rAAV vectors, and selected stably transfected lines by G418 resistance. Several of the transfected cell lines were analyzed for knockdown efficacy by Northern blot analysis and by ELISA assays.

### A) Construction of Anti-IL-1b Ribozyme Expressing Vectors

Hairpin ribozyme expression cassettes were synthesized by a PCR mutagenesis reaction using a double stranded DNA tetraloop ribozyme gene as a template ( . . . agaaNNNNACCAGAGAAACACACGGACTTCGGTCCGTGGTATATTACCTGGTA CGCGT . . . ), and a mutagenic oligonucleotide containing sequences for the 5′ end of the gene, including the target recognition sequences in the ribozyme, as a primer (GATATCGGATCCCAACAACTAGAACGGCACCAGAGAAACACACG).

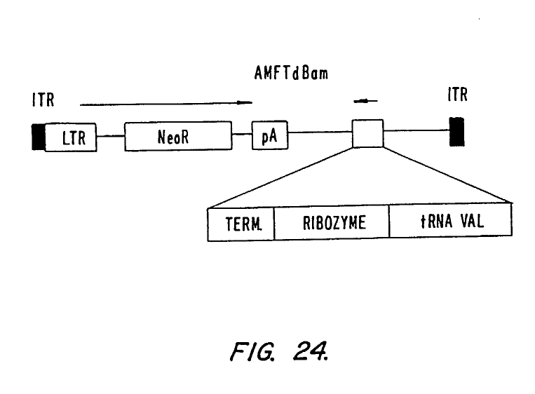

PCR products were digested with BamH1 and MluI restriction enzymes, which cleave at flanking, oligo-encoded sites, and cloned into BamH1, MluI digested pAMFTdBamHI (see FIG. 24).

### B) Transduction and Selection

rAAV vectors were prepared in A549 cells (162 cm²/vector) by transfection of the rAAV and AD8 helper plasmids, followed by infection with adenovirus. Cells were lysed 3 days later and clarified lysates were heated at 56° C. to inactivate the adenovirus. Crude lysates were directly used to transduce THP-1 cells. Transduced cell cultures were selected and maintained in media supplemented with 400 μg/ml G418.

### C) Ribozymes Reduce IL-1β Expression in THP-1 Cells

Northern blot analysis was performed to determine the relative levels of IL-1β RNA in ribozyme-expressing and control cells. The probe was prepared from RT-PCR fragments derived from THP-1 RNA (the RT-PCR primers used for probe preparation: sense 5′-CAGAAGTACCTGAGCTCGCCAGTGA-3′, anti-sense 5′-GCAGGCAGTTGGGCATTGGTGTAGA-3′), and the authenticity, of the fragments was confirmed by multiple restriction digests. The probe was labeled by random priming using the DNA Labeling kit (Pharmacia), and free nucleotides were removed by spin column. As quantified in Table 15, numerous anti-IL-1β ribozymes significantly reduced target IL-1β mRNA levels in THP-1 cells. The degree of mRNA reduction ranged from 45% to 99%.

TABLE 15
Percent reduction of IL-1β mRNA in transduced THP-1 cells.

| Ribozyme | % Reduction |
|---|---|
| ILβ-13 | 53 |
| ILβ195 | 99 |
| ILβ408 | 89 |
| ILβ801 | 45 |
| ILβ830 | 53 |
| ILβ921 | 71 |

To ascertain whether the observed reduction of IL-1β mRNAs resulted in lower IL-1β protein levels, supernatants from transduced cell cultures were examined for IL-1β protein levels by ELISA (R&D systems). IL-1β expression was induced by exposing THP-1 cells to 0, 10, or 100 ng/ml LPS in culture for 5-24 hours, as indicated. Supernatants were harvested and the remaining cells removed by centrifugation. As shown in Table 16, cultures which had the greatest ribozyme-mediated reduction of IL-1β mRNA produced the lowest amount of IL-1β protein. For example, ribozyme ILβ195, which produced a 99% reduction in IL-1β mRNA levels, caused a 62%, 92%, and 89% reduction in IL-1β protein levels at 0, 10, and 100 ng/ml LPS, respectively, and ribozyme IL1β408, which caused an 89% reduction in mRNA levels, created an 88%, 85%, and 86% reduction in protein levels at 0, 10, and 100 ng/ml LPS.

TABLE 16
Percent reduction IL-1β in transduced THP-1 cultures.

| | LPS concentration | | |
|---|---|---|---|
| Ribozyme | 0 | 10 ng/ml | 100 ng/ml |
| ILβ-13 | 70 ± 14 | 68 ± 5.7 | 77 ± 3.4 |
| ILβ-195 | 61 ± 10 | 92 ± 2.4 | 89 ± 1.8 |
| ILβ-408 | 88 ± 5.2 | 85 ± 1.6 | 86 ± 2.3 |
| ILβ-801 | 70 ± 5.2 | 26 ± 3.9 | 30 ± 7.8 |
| ILβ-830 | 67 ± 11 | 65 ± 3.9 | 59 ± 3.9 |
| ILβ-921 | 39 ± 14 | 64 ± 3.9 | 59 ± 2.7 |

### Example 23

### IL-1β Converting Enzyme (ICE) Knockdown in THP-1 Cells

IL-1β Convertase (ICE) is an intracellular protease that cleaves the precursor of IL-1β thereby creating the mature extracellular form of the protein. Ribozymes against ICE were cloned into AMFT vector and rAAV vectors were used to transduce the ribozymes into THP-1 cells. Transduced cells were selected using G418, as in Example 1. ICE mRNA levels were assessed by Northern blot analysis, using RT-PCR generated probes (sense 5′-GACCCGAGCTTTGATTGACTCCGT-3′, antisense 5′-GGTGGGCATCTGCGCTCTAGGA-3′). The Northern blot and phosphorimage analysis of this experiment was quantified as shown in Table 17. Multiple ribozymes significantly reduced ICE mRNA levels. The greatest reduction was seen with ribozyme ICE13, which produced a 94% reduction in ICE mRNA levels.

TABLE 17
Percent reduction of ICE mRNA in transduced THP-1 cells

| Ribozyme | % Reduction |
|---|---|
| ICE13 | 94 |
| ICE397 | 32 |
| ICE444 | 25 |
| ICE474 | 42 |
| ICE488 | 54 |
| ICE705 | 11 |
| ICE754 | 0 |
| ICE1236 | 67 |
| ICE1284 | 65 |

To determine if reductions of ICE mRNA resulted in lower ICE protein levels, ICE protein levels were measured by Western blot. Results of the western blot indicated that there is indeed a correlation between mRNA and protein levels in these cells.

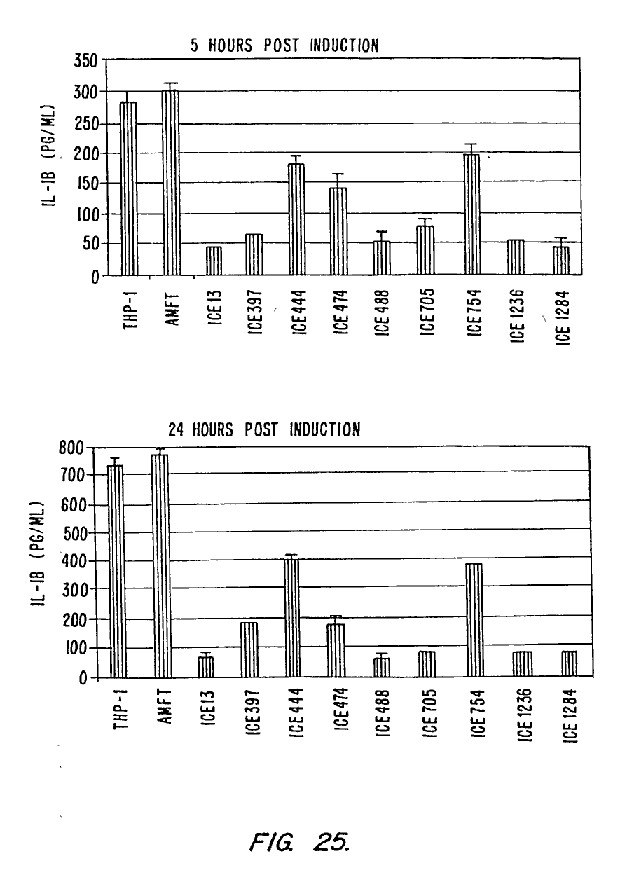

The function of ICE is to cleave IL-1β, thereby converting it from an intracellular to an extracellular form. Therefore, ribozyme-mediated reductions in ICE protein levels should result in the commensurate reduction of extracellular IL-1β. Consequently, measuring extracellular IL-1β levels should provide an accurate measure of ICE activity. Transduced cultures were induced with LPS (100 ng/ml), and supernatants were harvested at 5 and 24 hours post induction. Supernatants were centrifuged to remove any remaining cells, and IL-1β levels were assessed by ELISA. As shown in Table 18 and FIG. 25, extracellular IL-1β levels were reduced in all of the cultures, with reductions greater than 80% in many cases.

TABLE 18
Percent reduction of IL-1β in THP-1 cultures (ICE RZs)

| Ribozyme | % Reduction |
|---|---|
| ICE13 | 86 ± 0.7 |
| ICE397 | 79 ± 1.2 |
| ICE444 | 41 ± 4.7 |
| ICE474 | 54 ± 7.0 |
| ICE488 | 83 ± 5.8 |
| ICE705 | 74 ± 3.5 |
| ICE754 | 37 ± 5.8 |
| ICE1236 | 83 ± 1.2 |
| ICE1284 | 86 ± 5.4 |

### Example 24

### Knockdown of CCR-5

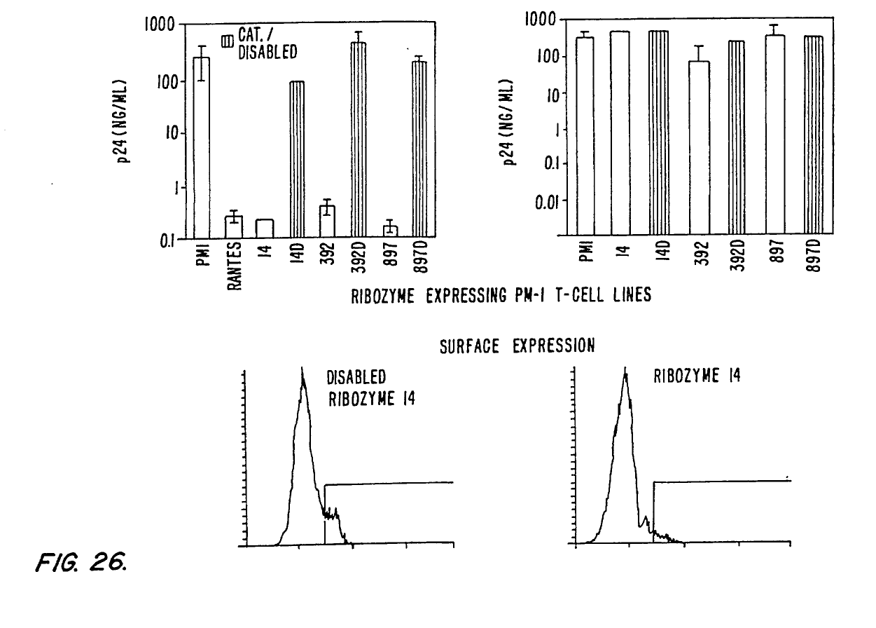

We have developed ribozymes against the HIV co-receptor, C-C chemokine receptor 5, and demonstrated their effectiveness in reducing CCR-5 mRNA and protein expression levels. We have also demonstrated that these ribozymes can reduce the susceptibility of T-cells to infection by macrophage tropic strains of HIV. The level of surface expression for CCR-5 was reduced when an active, but not a catalytically disabled, form of ribozyme 14 was expressed. Surface levels were assessed by FACS analysis. To determine whether this reduction of CCR-5 expression decreased the susceptibility of these cells to HIV infection, HIV levels (as measured by p24 levels) were determined following expression of ribozymes specific to CCR-5. As shown in FIG. 26, ribozymes specific to CCR-5 produced a marked reduction in the production of the CCR-5 tropic strain of HIV, HIV_{BaL}, in transduced PM-1 (Human T-cell line) cultures. HIV production was not inhibited, however, when a catalytically disabled form of the ribozyme (indicated by the suffix D) was used. To further confirm the specificity of this effect, we monitored whether these ribozymes were capable of inhibiting production of the CXCr-4 tropic virus, HIV_{IIIB}. Expression of the CCR-5 specific ribozymes produced no reduction in HIV production.

### Example 25

### Rapid Drug Selection

Only a fraction of a transfected or transduced population of cells will actually incorporate and express the introduced DNA. Accordingly, the separation of ribozyme-expressing from non-expressing cells is an important issue in target validation studies. By obtaining a uniformly expressing population of cells, changes in phenotype can be monitored with greatly increased sensitivity. Various methods can be employed to accomplish this task in a rapid and high throughput mode.

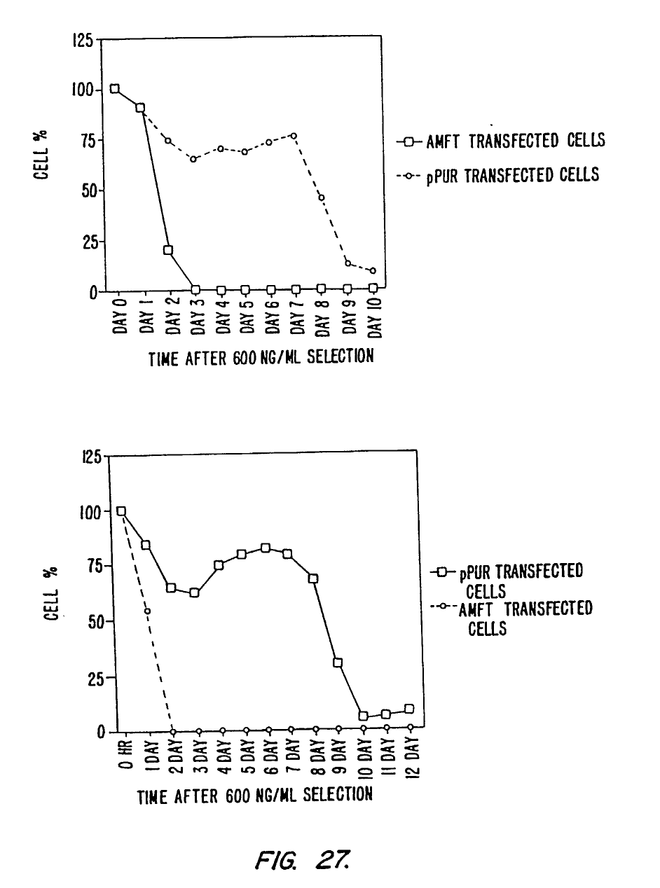

Drug selection can be employed to kill cells which do not receive a ribozyme expression vector delivered by transfection or transduction. Drug selection is typically used to obtain cells which stably express the drug resistance gene; however, we have found conditions under which cells transfected with puromycin-expressing vectors can survive for several days in the presence of puromycin, even when not stably transfected or transduced. During the same time period, untransfected cells are rapidly killed by the drug. Plasmids encoding puromycin resistance genes (pPur) were transfected into A549 and HeLa cells, and 600 ng/ml puromycin was added to the culture medium. As a control, cells were transfected with plasmids lacking puromycin resistance genes (AMFT). The number of cells was determined at 1-day intervals following transfection. Cells lacking the puromycin resistance gene were killed within 2 or 3 days after the addition of puromycin, whereas cells receiving puromycin resistance genes survived as long as 7-9 days (FIG. 27). Target validation could therefore be performed on these transiently transfected cells between 2-9 days following puromycin selection. Other drugs which rapidly kill cells can also be employed in this type of experiment.

### Example 26

### Co-selection for Overexpression of Ribozyme

It is critical for successful ribozyme gene knockdown experiments that the subject cells uniformly express ribozymes. In most systems, a pool of transduced or transfected cells are analyzed, and only a fraction of the cells are transfected in a given experiment. Consequently, any assay involving the cells will involve both expressing and non-expressing cells, and cells which express little to no ribozyme can contribute significant background even when using highly active ribozymes. To ensure that all cells are expressing the ribozyme, we co-expressed a ribozyme and a selectable marker, GFP, on the same mRNA. Because the ribozyme and the sequence encoding the GFP protein are present on the same mRNA, GFP expression provides an accurate marker of ribozyme expression. Numerous methods exist for detecting GFP expression, including spectrophotometry, fluorescence microscopy, and FACS. Furthermore, because we can differentially FACS for cells that express abundant amounts of GFP, we can enrich for a subpopulation that expresses very high amounts of ribozyme; these highly expressing cells will therefore increase the knockdown effect. Other marker genes could be linked to ribozyme expression in a similar manner, including genes conferring drug resistance.

### Example 27

### Rapid Selection of Ribozyme Expressing Cells by Expression of Cell Surface Markers

Selection of ribozyme expressing cells by G418 resistance takes approximately 2-4 weeks' time. Reducing this selection period would increase the speed of target validation analysis and allow the rapid detection of phenotypic changes; it would also allow the detection of phenotypes that change, or disappear, during ex vivo cultivation of primary cells. The use of vector-encoded cell surface proteins would allow the rapid selection of transduced or transfected cells by means of antibody or ligand capture of expressing cells. For example, a ribozyme and cell surface marker are encoded on the same mRNA. A population of cells is transfected with a construct encoding the mRNA. Cells expressing the surface marker are purified using one of a variety of differential selection schemes, e.g., FAC sorting, magnetic beads, or fixed ligand binding. A variety of marker proteins can be used including natural or altered versions of cell surface proteins, such as nerve growth factor receptor or single chain antibody molecules, e.g, as used in the pHOOK vector system (Clontech).

### Example 28

### PGK and tRNA Serine Promoters

In order to achieve effective target reduction in ribozyme-mediated validation experiments, promoter elements which drive the expression of ribozymes must be optimized. We have tested the efficacy of several ribozyme promoters in knockdown experiments against viral and cellular target RNAs. Two promoters, tRNAserine, and phosphoglycerate kinase (PGK), yielded reductions in target levels greater than or equal to the tRNAvaline promoter.

Promoter efficacy was measured by using them to express a ribozyme against the U5 region of HIV and measuring the resulting anti-HIV effect for each promoter. Table 19 shows results obtained using various RNA polymerase III promoters. Table 20 includes data generated by testing various RNA polymerase II promoters in a similar assay. The HIV protease inhibitor, indinavir, was included in these experiments as a positive control at 10 and 100 nM concentrations.

TABLE 19
Inhibition of HIV replication by U5 ribozyme
driven from RNA polymerase III promoters.

| | MOI 0.08 | | MOI 0.04 | | MOI 0.02 | |
|---|---|---|---|---|---|---|
| | % | | % | P-value | % | |
| | inhibition | P-value | inhibition | P-value | inhibition | P-value |
| AMFT | 69.2 ± 6.6 | | 88.7 ± 1.8 | | 96.6 ± 1.8 | |
| 10 nM | 61.6 ± 9.5 | 0.004 | 85.7 ± 4.0 | 0.015 | 94.7 ± 2.8 | 0.020 |
| 100 nM | 89.5 ± 1.2 | <0.001 | 95.9 ± 0.2 | <0.001 | 97.8 ± 1.7 | 0.132 |
| Serine | 82.0 ± 6.5 | 0.001 | 91.8 ± 2.2 | 0.012 | 96.6 ± 1.0 | 0.954 |
| Tryp | 63.0 ± 14 | 0.260 | 82.2 ± 4.2 | 0.002 | 93.5 ± 3.1 | 0.001 |
| Lysine | 52.9 ± 12 | <0.001 | 75.9 ± 6.6 | <0.001 | 93.1 ± 2.8 | <0.001 |
| Tyro- | 35.1 ± 26 | 0.002 | 82.2 ± 3.0 | <0.001 | 88.0 ± 2.8 | <0.001 |
| sine | | | | | | |
| Selano | 41.6 ± 13 | <0.001 | 72.2 ± 6.3 | <0.001 | 89.1 ± 5.9 | <0.001 |
| Alanine | 8.7 ± 24 | <0.001 | 71.0 ± 23 | 0.030 | 75.7 ± 21 | 0.005 |

Stable Molt4/8 cell lines were challenged in sextuplicate at the three MOIs indicated. Cultures were sampled for P24 production at 7 days post infection and results calculated as percent inhibition compared to ALNL-6 control. The SAVA was repeated and results of the two assays compiled in the above table. P values were obtained by t-Test (paired two-sample for means, two-tail) compared to AMFT.

Using the RNA polymerase III promoters, the highest inhibition was observed using tRNAserine, which produced a 82.0, 91.8, and 96.6% inhibition at 0.08, 0.04, and 0.02 MOI, respectively.

TABLE 20
Anti-HIV effect in cell culture using RNA pol II promoters.

| | MOI 0.08 | | MOI 0.04 | | MOI 0.02 | |
|---|---|---|---|---|---|---|
| | % | | % | | % | |
| | inhibition | P-value | inhibition | P-value | inhibition | P-value |
| AMFT | 75.0 ± 2.4 | | 76.1 ± 2.8 | | 83.9 ± 0.7 | |
| 10 nM | 57.8 ± 4.8 | 0.001 | 70.9 ± 2.1 | 0.009 | 81.3 ± 1.2 | 0.009 |
| 100 nM | 82.0 ± 1.2 | <0.001 | 86.1 ± 1.9 | <0.001 | 89.1 ± 0.5 | <0.001 |
| PGK | 90.3 ± 0.5 | 0.001 | 89.3 ± 0.4 | <0.001 | 89.2 ± 0.8 | <0.001 |
| CD11B | 88.2 ± 2.5 | <0.001 | 89.8 ± 0.9 | <0.001 | 91.6 ± 0.3 | <0.001 |
| CMV | 40.9 ± 1.5 | <0.001 | 65.0 ± 5.7 | <0.012 | 79.2 ± 7.3 | 0.167 |
| CD11A | 33.3 ± 6.7 | <0.001 | 42.7 ± 6.5 | <0.001 | 76.9 ± 1.0 | <0.001 |
| SV40 | 45.8 ± 10 | 0.002 | 55.7 ± 3.3 | <0.001 | 55.1 ± 10 | <0.001 |

Stable Molt4/8 cell lines were challenged in sextuplicate at the three MOIs indicated. Cultures were sampled for P24 production at 7 days post infection and results calculated as percent inhibition compared to ALNL-6 control. P values were obtained by t-Test (paired two-sample for means, two-tail) compared to AMFT.

As shown in Table 20 above, for the RNA polymerase II promoters, the highest inhibition was observed using the PGK promoter, which produced and 90.3, 89.3, and 89.2% inhibition for the same respective MOIs.

### Example 29

### 5′ and 3′ Auxiliary Sequences

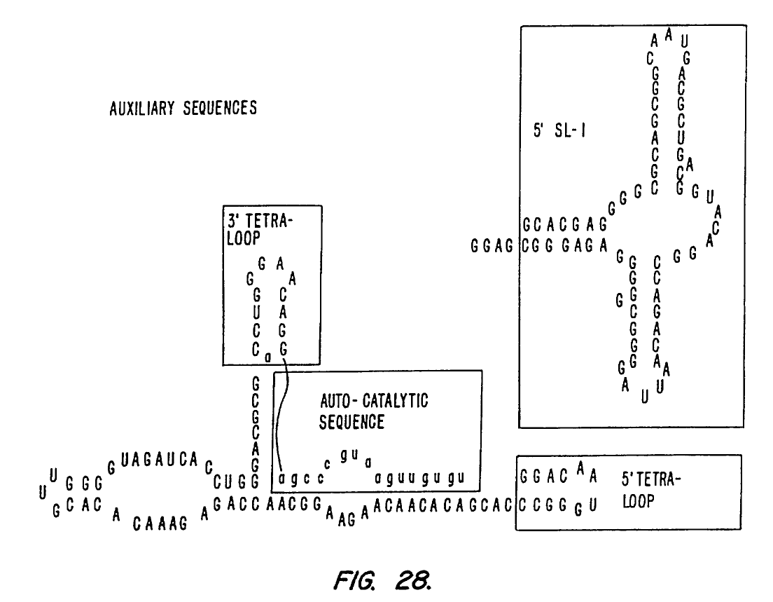

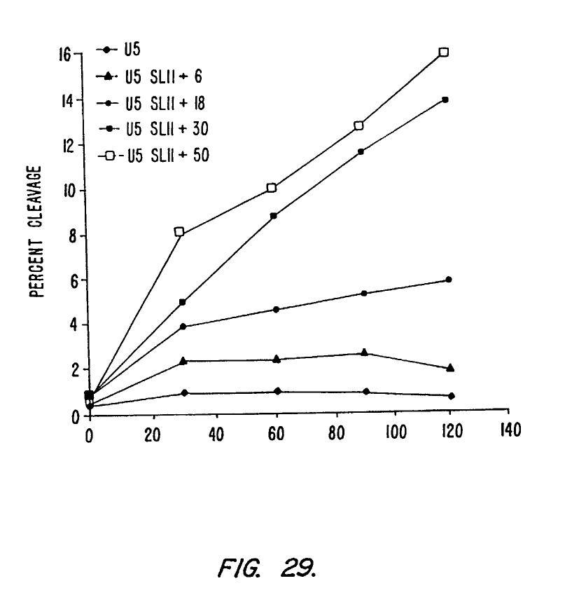

We have discovered that the activity of ribozymes can be enhanced by the addition of additional RNA sequences to the 5′ or 3′ terminus of the ribozyme (FIG. 28). In one example, we added the stem loop II region of the HIV rev responsive element, along with varying lengths of intervening sequence (from 0-50 nucleotides), to the 5′ end of the U5 ribozyme. We measured the activity of these ribozymes by in vitro time course cleavage reactions. As shown in FIG. 29, the addition of the stem loop II region, along with 50 bases of intervening sequence, produced a ribozyme with greater activity than the unmodified U5 ribozyme.

We also created a ribozyme with various 3′ structures which have greater activity than the unmodified ribozyme. One such structure consists of a tetraloop RNA sequence, along with several intervening bases, added to the 3′ end of a ribozyme. As shown in Table 21, a U5 ribozyme with a 3′ tetraloop RNA and a 6 base intervening spacer showed more than 2.5 times activity than the original ribozyme. We also created a 3′ tetralooped ribozyme that is followed by a substrate sequence. This autocatalytic ribozyme can efficiently cleave at the substrate sequence. Such self-cleaved ribozyme molecules, with an 8-base spacer between the tetraloop and the substrate sequence, are as active as the unmodified ribozyme.

TABLE 21
Effects of various 3′ auxiliary sequences on ribozyme activity.

| Sequence | Spacer | % U5 activity in vitro |
|---|---|---|
| U5 | | 100 |
| 3′ Tetraloop | 6 | 265 |
| | 7 | 119 |
| 3′ Tetraloop with autocat seq. | 6 | |
| | 8 | 103 |
| | 10 | 73 |
| | 12 | 81 |

### Example 30

### Partial Purification of rAAV

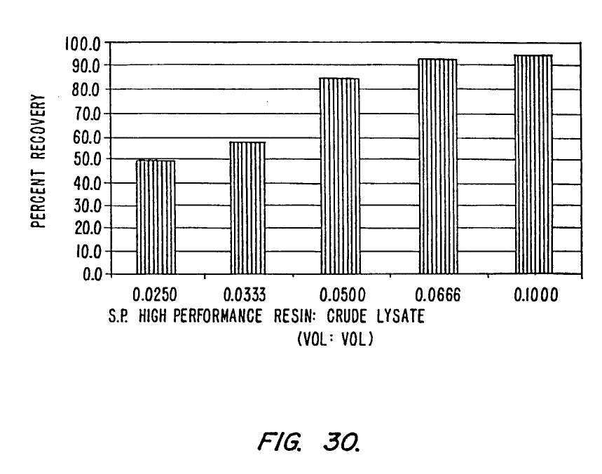

rAAV vectors can be partially purified from crude cell lysate preparations by rapid purification chromatographic methods. For example, we have used SP sepharose High Performance resin (Pharmacia) to rapidly concentrate and partially purify rAAV with high recovery rates. In these experiments, rAAV lysates were mixed with resin at 25° C. for 10 minutes, and the resin was recovered by centrifugation. The resin was washed twice with PBS+5 mM MgCl₂ by resuspension, followed by centrifugation. rAAV was then eluted in 400 mM NaCl, 1% glycerol, 5 mM MgCl₂. Two elutions in one bed volume of buffer were performed and eluates were combined. Using this method, greater than 80% recovery was achieved with a 1:20 ratio of resin to crude lysate (see, FIG. 30). This method can also be coupled with other chromatographic methods to achieve even greater purification and concentration. For example, POROS 50HQ resin (Perceptive Biosystems) could be used in series with the previously described technique. One such method would entail the application of POROS 50HQ to crude lysate to bind various proteins and macromolecules, including contaminating adenovirus. rAAV does not typically bind and can be recovered by separation of resin from the crude preparation by centrifugation or other methods. This “eluate” could then be applied to SP sepharose, and rAAV purified by methods described above.

### Example 31

### Multi-ribozyme Vectors

To enhance the efficiency of target validation methods, or to address the consequences of simultaneously reducing the expression of multiple gene targets, multiple ribozymes can be included in the same vector and simultaneously expressed in the same cell. These multiple ribozymes can be encoded on a single mRNA, to ensure that they are always expressed at similar levels.

TABLE 22
Multi-ribozyme vectors.

| | MOI 0.08 | | MOI 0.04 | | MOI 0.02 | |
|---|---|---|---|---|---|---|
| | % | | % | | % | |
| | inhibition | P-value | inhibition | P-value | inhibition | P-value |
| AMFT | 76.6 ± 4.1 | | 85.3 ± 1.6 | | 94.4 ± 2.6 | |
| 10 nM | 68.8 ± 4.5 | 0.003 | 73.9 ± 3.6 | <0.001 | 74.8 ± 1.5 | <0.001 |
| 100 nM | 93.3 ± 0.4 | <0.001 | 95.5 ± 0.6 | <0.001 | 98.5 ± 0.5 | 0.003 |
| TF-1.1 | 88.2 ± 3.0 | 0.009 | 92.4 ± 1.4 | <0.001 | 97.5 ± 1.8 | 0.013 |
| AS | | | | | | |
| TF-1.1 | 83.0 ± 2.7 | 0.018 | 88.3 ± 2.2 | 0.005 | 86.5 ± 6.1 | 0.038 |
| S | | | | | | |

Stable Molt4/8 cell lines were challenged in sextuplicate at the three MOIs indicated. Cultures were sampled for p24 production at 76 days post infection and results calculated as percent inhibition compared to ALNL-6 control.. P values were obtained by t-Test (paired two-sample for means, two-tail) compared to AMFT.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes. This application is related to PCT application No: PCT/US98/01196, filed on Feb. 24, 1998, which is a continuation of U.S. Ser. No. 60/037,352, filed on Jan. 23, 1997, both of which are incorporated by reference in their entirety for all purposes.

What is claimed is:

1. A substantially complete ribozyme library comprising a collection of adeno-associated virus (AAV), retroviral, or Eppstein-Barr virus (EBV) vectors, or a collection of retroviral vectors containing nucleic acids encoding hairpin ribozymes in expression cassettes wherein said collection of AAV, retroviral, or EBV vectors contains nucleic acids encoding on average about 90% or more of all possible hairpin ribozyme binding sequences having eight or more randomized nucleotides.

2. The ribozyme library of claim 1, wherein said collection of vectors contains nucleic acids encoding about 95% or more of all possible hairpin ribozyme binding sequences.

3. The ribozyme library of claim 1, wherein said collection of vectors contains nucleic acids encoding about 95% or more of all possible hairpin ribozyme binding sequences having 9 or more randomized nucleotides.

4. The ribozyme library of claim 1, wherein said collection of vectors contains nucleic acids encoding about 95% or more of all possible hairpin ribozyme binding sequences having 12 randomized nucleotides.

5. The ribozyme library of claim 1, wherein said nucleic acids are plasmids.

6. The ribozyme library of claim 1, wherein said library contains no toxic ribozymes.

7. The ribozyme library of claim 1, wherein said collection of vectors is a collection of AAV vectors.

8. The ribozyme library of claim 7, wherein said nucleic acids comprise a pair of inverted terminal repeats (ITRs) of adeno-associated viral genome.

9. The ribozyme library of claim 1, wherein said nucleic acids comprise a selectable marker.

10. The ribozyme library of claim 9, wherein said selectable marker is selected from the group consisting of Neo^{r}, amd Hygro^{r}.

11. The ribozyme library of claim 10, wherein said selectable marker is operably linked to an SV40 promoter.

12. The ribozyme library of claim 1, wherein the ribozyme-encoding nucleic acid is operably linked to a tRNA promoter.

13. The ribozyme library of claim 1, wherein the ribozyme-encoding nucleic acid is operably linked to a promoter selected from the group consisting of tRNAval, tRNAser, and PGK.

14. A substantially complete ribozyme gene library comprising a collection of plasmids wherein members of said collection encode a retroviral, adeno-associated virus (AAV), or Epstein Barr virus (EBV) vector containing a ribozyme-encoding nucleic acid and said collection of plasmids encodes on average about 90% or more of all possible hairpin ribozyme binding sequences having eight or more randomized nucleotides.

15. The ribozyme gene library of claim 14, wherein said collection of plasmids encodes on average about 95% or more of all possible hairpin ribozyme binding sequences.

16. The ribozyme gene library of claim 14, wherein said collection of plasmids encodes on average about 95% or more of all possible hairpin ribozyme binding sequences having 9 or more randomized nucleotides.

17. The ribozyme gene library of claim 14, wherein said library contains essentially no toxic ribozymes.

18. The ribozyme gene library of claim 14, wherein members of said collection encode an AAV vector.

19. The ribozyme gene library of claim 18, wherein said nucleic acids comprise a pair of inverted terminal repeats (ITRs) of adeno-associated viral genome.

20. The ribozyme gene library of claim 14, wherein said plasmids contain a selectable marker.

21. The ribozyme gene library of claim 20, wherein said selectable marker is selected from the group consisting of Neo^{r}, and Hygro^{r}.

22. The ribozyme gene library of claim 21, wherein said selectable marker is operably linked to an SV40 promoter.

23. The ribozyme gene library of claim 14, wherein the ribozyme-encoding nucleic acid is operably linked to a tRNA promoter.

24. The ribozyme gene library of claim 14, wherein the ribozyme-encoding nucleic acid is operably linked to a promoter selected from the group consisting of tRNAval, tRNAser, and PGK.

25. A method of selecting a ribozyme that specifically binds and cleaves a nucleic acid target, said method comprising:
i) transfecting a population of cells with a substantially complete hairpin ribozyme library comprising a collection of adeno-associated virus (AAV), retroviral, or Epstein Barr virus (EBV) vectors containing nucleic acids encoding hairpin ribozymes in expression cassettes wherein said collection of AAV, retroviral, or EBV vectors contains nucleic acids encoding on average about 90%or more of all possible hairpin ribozyme binding sequences having eight or more randomized nucleotides;
ii) detecting a phenotypic difference between a transfected cell that expresses at least one hairpin ribozyme encoded by said library and a control cell lacking an active members of said ribozyme library, wherein said phenotypic difference is a consequence of cleavage of said target; and
iii) recovering a ribozyme associated with said phenotypic difference.

26. The method of claim 25, wherein said transfecting produces a population of cells stably transfected with an expression cassette encoding a hairpin ribozyme.

27. The method of claim 25, wherein said hairpin ribozyme is constitutively expressed.

28. The method of claim 25, wherein said recovering comprises isolating a multiplicity of ribozymes to produce a targeted ribozyme library.

29. The method of claim 28, further comprising
iv) transfecting a population of cells with said targeted ribozyme library;
v) detecting a phenotypic difference between a transfected cell that expresses at least one hairpin ribozyme encoded by said targeted ribozyme library and a control cell lacking an active member of said ribozyme library, wherein said phenotypic difference is a consequence of cleavage of said target; and
vi) recovering a ribozyme associated with said phenotypic difference.

30. The method of claim 25, wherein said recovering comprises isolating and sequencing the binding site of said ribozyme.

31. The method of claim 30, further comprising providing a probe that hybridizes to the nucleic acid specifically bound by said ribozyme.

32. The method of claim 31, wherein said probe is labeled.

33. The method of claim 25, wherein phenotypic difference is a difference in transcription or expression of a reporter gene or cDNA.

34. The method of claim 25, wherein phenotypic difference is the ability of a cell to grow on soft agar.

35. The method of claim 25, wherein phenotypic difference is the ability of a cell to differentiate.

36. The method of claim 35, wherein said ability to differentiate is identified by the adherence of the cell to a surface in culture.

37. The method of claim 25, wherein said phenotypic difference is resistance to a drug.

38. The method of claim 37, wherein said drug is selected from the group consisting of cisplatin, doxirubicin, taxol, camptothecin, daunorubicin, and methotrexate.

39. The method of claim 25, wherein said phenotypic difference is a change in the expression level of a reporter gene linked to a gene whose regulation it is desired to alter.

40. The method of claim 25, wherein said collection of AAV, retroviral, or EBV vectors contains nucleic acids encoding on average about 95% or more of all possible hairpin ribozyme binding sequences.

41. The method of claim 25, wherein said collection of AAV, retroviral, or EBV vectors contains nucleic acids encoding on average about 90% or more of all possible hairpin ribozyme binding sequences having 9 or more randomized nucleotides.

42. The method of claim 25, wherein said nucleic acids are plasmids.

43. The method of claim 25, wherein said library contains no toxic ribozymes.

44. The method of claim 25, wherein said collection of vectors is a collection of AAV vectors.

45. The method of claim 44, wherein said nucleic acids comprise a pair of inverted terminal repeats (ITRs) of adeno-associated viral genome.

46. The method of claim 25, wherein said nucleic acids comprise a selectable marker.

47. The method of claim 46, wherein said selectable marker is selected from the group consisting of Neo^{r} and Hygro^{r}.

48. The method of claim 47, wherein said selectable marker is operably linked to an SV40 promoter.

49. The method of claim 25, wherein the ribozyme-encoding nucleic acid is operably linked to a tRNA promoter.

50. The method of claim 25, wherein the ribozyme-encoding nucleic acid is operably linked to a promoter selected from the group consisting of tRNAval, tRNAser, and PGK.

51. A method of identifying a gene or mRNA altered expression of which results in alteration of a detectable phenotypic character, said method comprising:
i) stably transfecting a population of cells with a hairpin ribozyme library comprising a collection of adeno-associated virus (AAV) vectors containing nucleic acids encoding hairpin ribozymes in expression cassettes;
ii) detecting a phenotypic difference between a transfected cell that expresses said hairpin ribozyme and a control cell lacking an active form of said hairpin ribozyme;
iii) recovering a ribozyme associated with said phenotypic difference; and
iv) sequencing the binding site sequence of the recovered ribozyme to identify the nucleic acid to which it bound.

52. The method of claim 51, wherein said hairpin ribozyme is constitutively expressed.

53. The method of claim 51, wherein said ribozyme library is a substantially complete ribozyme library.

54. The method of claim 51, wherein said ribozyme library is a targeted ribozyme library.

55. The method of claim 51, wherein said recovering comprises reverse transcribing and amplifying the nucleic acid comprising said ribozyme.

56. The method of claim 55, further comprising providing a probe that hybridizes to the nucleic acid specifically bound by said ribozyme.

57. The method of claim 56, wherein said probe is labeled.

58. The method of claim 51, wherein said phenotypic difference is a difference in transcription or expression of a reporter gene or cDNA.

59. The method of claim 51, wherein said phenotypic difference is the ability of a cell to grow on soft agar.

60. The method of claim 51, wherein said phenotypic difference is the ability of a cell to differentiate.

61. The method of claim 60, wherein said ability to differentiate is identified by the adherence of the cell to a surface in culture.

62. The method of claim 51, wherein phenotypic difference is resistance to a drug.

63. The method of claim 62, wherein said drug is selected from the group consisting of cisplatin, doxirubicin, taxol, camptothecin, daunorubicin, and methotrexate.

64. The method of claim 51, wherein said phenotypic difference is a change in the expression level of a reporter gene linked to a gene whose regulation it is desired to alter.

65. A method of producing a cell line having altered expression of a gene said method comprising stably transfecting a cell with a vector encoding a hairpin ribozyme wherein said hairpin ribozyme is identified according to the method of claim 25.

66. A population of mammalian cells containing a substantially complete ribozyme library comprising a collection of adeno-associated virus (AAV), retrovirus, or Epstein Barr virus (EBV) vectors containing nucleic acids encoding hairpin ribozymes in expression cassettes wherein said collection of AAV, retroviral, or EBV vectors contains nucleic acids encoding on average about 90% or more of all possible hairpin ribozyme binding sequences having eight or more randomized nucleotides.

67. The ribozyme library of claim 66, wherein said collection of AAV, retroviral, or EBV vectors contains nucleic acids encoding about 95% or more of all possible hairpin ribozyme binding sequences.

68. The ribozyme library of claim 66, wherein said collection of AAV, retroviral, or EBV vectors contains nucleic acids encoding about 95% or more of all possible hairpin ribozyme binding sequences having 9 or more randomized nucleotides.

69. The ribozyme library of claim 66, wherein said collection of AAV, retroviral, or EBV vectors contains nucleic acids encoding about 95% or more of all possible hairpin ribozyme binding sequences having 12 randomized nucleotides.

70. A kit comprising one or more containers containing
a substantially complete ribozyme library comprising a collection of adeno-associated virus (AAV), retrovirus, or Epstein Barr virus (EBV) vectors containing nucleic acids encoding hairpin ribozymes in expression cassettes wherein said collection of AAV, retroviral, or EBV vectors contains nucleic acids encoding on average about 90% or more of all possible hairpin ribozyme binding sequences having eight or more randomized nucleotides; or
a substantially complete ribozyme gene library comprising a collection of plasmids wherein members of said collection encode a retroviral, adeno-associated virus (AAV), or Epstein Barr virus (EBV) vector containing a ribozyme-encoding nucleic acid and said collection of plasmids encodes on average about 90% or more of all possible hairpin ribozyme binding sequences having eight or more randomized nucleotides.
